SEQUENCE LISTING (118 sequences)

SEQ ID NO: 1 - protein, 175 aa, Homo sapiens
MSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 2 - protein, 27 aa, Artificial Sequence
  Linker
GGGGSGGGGSGGGGSGGGGSGGGGSGG

SEQ ID NO: 3 - protein, 326 aa, Artificial Sequence
  VHH-hFerr
QVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTVVSEWDYDYDYWGQGTQVTVSGGGGSGGGGSGGGGSGGGGSGGGGSGGGSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 4 - protein, 354 aa, Artificial Sequence
  VHH-Fc
QVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTVVSEWDYDYDYWGQGTQVTVSSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 90 aa, Artificial Sequence
  N-hFerritinLC
MSSQIRQNYSTDVEAAVNSLVNLYLQASYTYLSLGFYFDRDDVALEGVSHFFRELAEEKREGYERLLKMQNQRGGRALFQDIKKPAEDEW

SEQ ID NO: 6 - protein, 85 aa, Artificial Sequence
  C-hFerritinLC
GKTPDAMKAAMALEKKLNQALLDLHALGSARTDPHLCDFLETHFLDEEVKLIKKMGDHLTNLHRLGGPEAGLGEYLFERLTLRHD

SEQ ID NO: 7 - protein, 29 aa, Artificial Sequence
  Signal sequence
MGILPSPGMPALLSLVSLLSVLLMGCVAE

SEQ ID NO: 8 - protein, 70 aa, Artificial Sequence
  Linker
GGGGSGGGGSGGGGSGGGGSGGGGSGGGGSGGGGSGGGGSGGGGSGGGGSGGGGSGGGGSGGGGSGGGGS

SEQ ID NO: 9 - protein, 27 aa, Artificial Sequence
  Linker
GGGGSGGGGSGGGGSGGGGSGGGGSGG

SEQ ID NO: 10 - protein, 714 aa, Artificial Sequence
  BD23-scFab-hFerritinLC
DIQMTQSPSTLSASVGDRVTITCRASQSISSWLAWYQQKPGKAPKLLIYKASSLESGVPSRFSGSGSGTEFTLTISSLQPDDFATYYCQQYNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSGGGGSGGGGSGGGGSGGGGSQVQLVQSGSELKKPGASVKVSCKASGYTFTSYAMNWVRQAPGQGLEWMGWINTNTGNPTYAQGFTGRFVFSLDTSVSTAYLQISSLKAEDTAVYYCARPQGGSSWYRDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 11 - protein, 107 aa, Artificial Sequence
  VK for BD23
DIQMTQSPSTLSASVGDRVTITCRASQSISSWLAWYQQKPGKAPKLLIYKASSLESGVPSRFSGSGSGTEFTLTISSLQPDDFATYYCQQYNSYPYTFGQGTKLEIK

SEQ ID NO: 12 - protein, 126 aa, Artificial Sequence
  VH for BD23
QVQLVQSGSELKKPGASVKVSCKASGYTFTSYAMNWVRQAPGQGLEWMGWINTNTGNPTYAQGFTGRFVFSLDTSVSTAYLQISSLKAEDTAVYYCARPQGGSSWYRDYYYGMDVWGQGTTVTVSS

SEQ ID NO: 13 - protein, 627 aa, Artificial Sequence
  BD23-scFab-C_hFerritinLC
LEDIQMTQSPSTLSASVGDRVTITCRASQSISSWLAWYQQKPGKAPKLLIYKASSLESGVPSRFSGSGSGTEFTLTISSLQPDDFATYYCQQYNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSGGGGSGGGGSGGGGSGGGGSQVQLVQSGSELKKPGASVKVSCKASGYTFTSYAMNWVRQAPGQGLEWMGWINTNTGNPTYAQGFTGRFVFSLDTSVSTAYLQISSLKAEDTAVYYCARPQGGSSWYRDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSGGGGSGGGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 14 - protein, 640 aa, Artificial Sequence
  scFc-N_hFerritinLC
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEW

SEQ ID NO: 15 - protein, 227 aa, Artificial Sequence
  scFc(LALAP)
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 721 aa, Artificial Sequence
  4A8-scFab-hFerritinLC
EIVMTQSPLSSPVTLGQPASISCRSSQSLVHSDGNTYLSWLQQRPGQPPRLLIYKISNRFSGVPDRFSGSGAGTDFTLKISRVEAEDVGVYYCTQATQFPYTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSGGGGSGGGGSGGGGSGGGGSEVQLVESGAEVKKPGASVKVSCKVSGYTLTELSMHWVRQAPGKGLEWMGGFDPEDGETMYAQKFQGRVTMTEDTSTDTAYMELSSLRSEDTAVYYCATSTAVAGTPDLFDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 17 - protein, 112 aa, Artificial Sequence
  VK for 4A8
EIVMTQSPLSSPVTLGQPASISCRSSQSLVHSDGNTYLSWLQQRPGQPPRLLIYKISNRFSGVPDRFSGSGAGTDFTLKISRVEAEDVGVYYCTQATQFPYTFGQGTKVDIK

SEQ ID NO: 18 - protein, 128 aa, Artificial Sequence
  VH for 4A8
EVQLVESGAEVKKPGASVKVSCKVSGYTLTELSMHWVRQAPGKGLEWMGGFDPEDGETMYAQKFQGRVTMTEDTSTDTAYMELSSLRSEDTAVYYCATSTAVAGTPDLFDYYYGMDVWGQGTTVTVSS

SEQ ID NO: 19 - protein, 632 aa, Artificial Sequence
  4A8-scFab C_hFerritinLC
EIVMTQSPLSSPVTLGQPASISCRSSQSLVHSDGNTYLSWLQQRPGQPPRLLIYKISNRFSGVPDRFSGSGAGTDFTLKISRVEAEDVGVYYCTQATQFPYTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSGGGGSGGGGSGGGGSGGGGSEVQLVESGAEVKKPGASVKVSCKVSGYTLTELSMHWVRQAPGKGLEWMGGFDPEDGETMYAQKFQGRVTMTEDTSTDTAYMELSSLRSEDTAVYYCATSTAVAGTPDLFDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSGGGGSGGGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 20 - protein, 183 aa, Artificial Sequence
  mFerritin
MTSQIRQNYSTEVEAAVNRLVNLHLRASYTYLSLGFFFDRDDVALEGVGHFFRELAEEKREGAERLLEFQNDRGGRALFQDVQKPSQDEWGKTQEAMEAALAMEKNLNQALLDLHALGSARTDPHLCDFLESHYLDKEVKLIKKMGNHLTNLRRVAGPQPAQTGAPQGSLGEYLFERLTLKHD

SEQ ID NO: 21 - protein, 747 aa, Artificial sequence
  Anti-CD19 single chain immunoglobulin
DILLTQTPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGSSGSGSGSTGTSSSGTGTSAGTTGTSASTSGSGSGGGGGSGGGGSAGGTATAGASSGSGSSGSSSSGGTGTGQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTSVTVSSSQSFPNVFPLVSCESPLSDKNLVAMGCLARDFLPSTISFTWNYQNNTEVIQGIRTFPTLRTGGKYLATSQVLLSPKSILEGSDEYLVCKIHYGGKNRDLHVPIPSKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 22 - protein, 734 aa, Artificial Sequence
  IgG2a Fc_mFerr
KPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGSSGSGSGSTGTSSSGTGTSAGTTGTSASTSGSGSGGGGGSGGGGSAGGTATAGASSGSGSSGSSSSGGTGKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKSRASTASSASSGGGGGGSGGSGGSGGSMTSQIRQNYSTEVEAAVNRLVNLHLRASYTYLSLGFFFDRDDVALEGVGHFFRELAEEKREGAERLLEFQNDRGGRALFQDVQKPSQDEWGKTQEAMEAALAMEKNLNQALLDLHALGSARTDPHLCDFLESHYLDKEVKLIKKMGNHLTNLRRVAGPQPAQTGAPQGSLGEYLFERLTLKHD

SEQ ID NO: 23 - protein, 640 aa, Artificial Sequence
  scFc-N-hFerr LALAP I253A
DKTHTCPPCPAPEAAGGPSVFLFPPKPKDTLMASRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGGGGSGGGGSGGGGSGGGGSGGGGSGGGGSDKTHTCPPCPAPEAAGGPSVFLFPPKPKDTLMASRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEW

SEQ ID NO: 24 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 214 aa, Artificial Sequence
  Antibody 56 light chain
DIQMTQSPSSLSASVGDRVTITCRASQGISSYLAWYQQKPGKAPKLLIYDASNLQSGVPSRFSGSGSGTDFTLTISSLQPEDFATYYCQQANSFPSTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 219 aa, Artificial Sequence
  Antibody 56 heavy chain
QVQLVQSGAEVKKPGASVKVSCKASGYTFTSYGISWVRQAPGQGLEWMGWISAYNGNTNYAQKLQGRVTMTRDTSTSTVYMELSSLRSEDTAVYYCARDIGPIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 27 - protein, 214 aa, Artificial Sequence
  Antibody 349 light chain
DIQMTQSPSSLSASVGDRVTITCRASQSISSWLAWYQQKPGKAPKLLIYDTSNLETGVPSRFSGSGSGTDFTLTISSLQPEDFATYYCQQSYTTPWTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 226 aa, Artificial Sequence
  Antibody 349 heavy chain
EVQLLESGGGLVQPGGSLRLSCAASGFTFSNYGMHWVRQAPGKGLEWVSGISSAGSITNYADSVKGRFTISRDNSKNTLYLQMNSLRAEDTAVYYCAGNHAGTTVTSEYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 29 - protein, 215 aa, Artificial Sequence
  Antibody 178 light chain
EIVMTQSPATLSVSPGERATLSCKASQSVSGTYLAWYQQKPGQAPRLLIYGASTRATGIPARFSGSGSGTEFTLTISSLQSEDFAVYYCLQTHSYPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 225 aa, Artificial Sequence
  Antibody 178 heavy chain
QVQLVQSGAEVKKPGASVKVSCKASGYTFTDYHMHWVRQAPGQGLEWMGWINPNSGGTNYAQKFQGRVTMTRDTSTSTVYMELSSLRSEDTAVYYCARDISSWYEITKFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 31 - protein, 214 aa, Artificial Sequence
  Antibody 108 light chain
DIQMTQSPSSLSASVGDRVTITCRASQVITNNLAWYQQKPGKAPKLLIYDASTLETGVPSRFSGSGSGTDFTLTISSLQPEDFATYYCQQSYTFPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 226 aa, Artificial Sequence
  Antibody 108 heavy chain
QVQLVQSGAEVKKPGASVKVSCKASGYIFSRYAIHWVRQAPGQGLEWMGWMNPISGNTDYAPNFQGRVTMTRDTSTSTVYMELSSLRSEDTAVYYCAKDGSQLAYLVEYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 33 - protein, 214 aa, Artificial Sequence
  Antibody 128 light chain
DIQMTQSPSSLSASVGDRVTITCRASQNISRYLNWYQQKPGKAPKLLIYDASNLETGVPSRFSGSGSGTDFTLTISSLQPEDFATYYCQQANGFPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 225 aa, Artificial Sequence
  Antibody 128 heavy chain
QVQLVQSGAEVKKPGASVKVSCKASGYTFTHYYMHWVRQAPGQGLEWMGIINPSSSSASYSQKFQGRVTMTRDTSTSTVYMELSSLRSEDTAVYYCARDGRYGSGSYPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 35 - protein, 214 aa, Artificial Sequence
  Antibody 160 light chain
DIQMTQSPSSLSASVGDRVTITCRASQSVSSWLAWYQQKPGKAPKLLIYAASSLQSGVPSRFSGSGSGTDFTLTISSLQPEDFATYYCQQGYTTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 224 aa, Artificial Sequence
  Antibody 160 heavy chain
QVQLVQSGAEVKKPGASVKVSCKASGYTFTGHDMHWVRQAPGQGLEWMGIINPSGGSTSYAQKFQGRVTMTRDTSTSTVYMELSSLRSEDTAVYYCARANSLRYYYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 37 - protein, 219 aa, Artificial Sequence
  Antibody 368 light chain
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPATFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 220 aa, Artificial Sequence
  Antibody 368 heavy chain
QVQLVQSGAEVKKPGSSVKVSCKASGYTFTSYDINWVRQAPGQGLEWMGAIMPMFGTANYAQKFQGRVTITADESTSTAYMELSSLRSEDTAVYYCARGSSGYYYGWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 39 - protein, 219 aa, Artificial Sequence
  Antibody 192 light chain
DIVMTQSPLSLPVTPGEPASISCRSSQSLLHSNGYNYLDWYLQKPGQSPQLLIYAASSLQSGVPDRFSGSGSGTDFTLKISRVEAEDVGVYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 224 aa, Artificial Sequence
  Antibody 192 heavy chain
QVQLVQSGAEVKKPGSSVKVSCKASGGTFSSYAISWVRQAPGQGLEWMGWINPNSGGANYAQKFQGRVTITADESTSTAYMELSSLRSEDTAVYYCSTYYYDSSGYSTDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 41 - protein, 214 aa, Artificial Sequence
  Antibody 158 light chain
DIQMTQSPSSLSASVGDRVTITCRASQSISRYLNWYQQKPGKAPKLLIYDASNLESGVPSRFSGSGSGTDFTLTISSLQPEDFATYYCQQANSFPLTFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 225 aa, Artificial Sequence
  Antibody 158 heavy chain
QVQLVQSGAEVKKPGASVKVSCKASGYTFTGYYMHWVRQAPGQGLEWMGWINPLNGGTNFAPKFQGRVTMTRDTSTSTVYMELSSLRSEDTAVYYCARDPGGSYSNDAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 43 - protein, 219 aa, Artificial Sequence
  Antibody 180 light chain
DIVMTQSPLSLPVTPGEPASISCRSSQSLLHSNGYNYLDWYLQKPGQSPQLLIYAASSLQSGVPDRFSGSGSGTDFTLKISRVEAEDVGVYYCQQYYSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 227 aa, Artificial Sequence
  Antibody 180 heavy chain
QVQLVQSGAEVKKPGSSVKVSCKASGYTFTSYAMHWVRQAPGQGLEWMGRISPRSGGTKYAQRFQGRVTITADESTSTAYMELSSLRSEDTAVYYCAREAVAGTHPQAGDFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 45 - protein, 215 aa, Artificial Sequence
  Antibody 254 light chain
DIQMTQSPSSLSASVGDRVTITCRASQGISSYLAWYQQKPGKAPKLLIYDASSLQIGVPSRFSGSGSGTDFTLTISSLQPEDFATYYCLQSYSTPPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 222 aa, Artificial Sequence
  Antibody 254 heavy chain
EVQLLESGGGLVQPGGSLRLSCAASGFTFSSSAMHWVRQAPGKGLEWVSAIGTGGDTYYADSVKGRFTISRDNSKNTLYLQMNSLRAEDTAVYYCAREGDGYNFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 47 - protein, 215 aa, Artificial Sequence
  Antibody 120 light chain
EIVMTQSPATLSVSPGERATLSCRASQSVSSRYLAWYQQKPGQAPRLLIYGASTRATGIPARFSGSGSGTEFTLTISSLQSEDFAVYYCQQYYTTPRTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 227 aa, Artificial Sequence
  Antibody 120 heavy chain
QVQLVQSGAEVKKPGASVKVSCKASGYTFTSYDINWVRQAPGQGLEWMGMIDPSGGSTSYAQKFQGRVTMTRDTSTSTVYMELSSLRSEDTAVYYCAKDFGGGTRYDYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 49 - protein, 214 aa, Artificial Sequence
  Antibody 64 light chain
DIQMTQSPSSLSASVGDRVTITCRASQGISSHLAWYQQKPGKAPKLLIYDASNLETGVPSRFSGSGSGTDFTLTISSLQPEDFATYYCQQTYSTPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 224 aa, Artificial Sequence
  Antibody 64 heavy chain
EVQLLESGGGLVQPGGSLRLSCAASGFPFSQHGMHWVRQAPGKGLEWVSAIDRSGSYIYYADSVKGRFTISRDNSKNTLYLQMNSLRAEDTAVYYCARDTYGGKVTYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 51 - protein, 220 aa, Artificial Sequence
  Antibody 298 light chain
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 222 aa, Artificial Sequence
  Antibody 298 heavy chain
QVQLVQSGAEVKKPGASVKVSCKASGGTFSTYGISWVRQAPGQGLEWMGWISPNSGGTDLAQKFQGRVTMTRDTSTSTVYMELSSLRSEDTAVYYCASDPRDDIAGGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 53 - protein, 214 aa, Artificial Sequence
  Antibody 82 light chain
DIQMTQSPSSLSASVGDRVTITCRASQVISNYLAWYQQKPGKAPKLLIYDASNLETGVPSRFSGSGSGTDFTLTISSLQPEDFATYYCQQSFSPPPTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 225 aa, Artificial Sequence
  Antibody 82 heavy chain
QVQLVQSGAEVKKPGASVKVSCKASGGSFSTSAFYWVRQAPGQGLEWMGWINPYTGGTNYAQKFQGRVTMTRDTSTSTVYMELSSLRSEDTAVYYCARSRALYGSGSYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 55 - protein, 214 aa, Artificial Sequence
  Antibody 46 light chain
DIQMTQSPSSLSASVGDRVTITCRASQSISSWLAWYQQKPGKAPKLLIYDASNLETGVPSRFSGSGSGTDFTLTISSLQPEDFATYYCQQSYSTPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 220 aa, Artificial Sequence
  Antibody 46 heavy chain
EVQLLESGGGLVQPGRSLRLSCAASGFTFSSYAMSWVRQAPGKGLEWVSTIYSGGSTYYADSVKGRFTISRDNSKNTLYLQMNSLRAEDTAVYYCARGDSRDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 57 - protein, 214 aa, Artificial Sequence
  Antibody 324 light chain
DIQMTQSPSSLSASVGDRVTITCRASQSITTYLNWYQQKPGKAPKLLIYDASNLETGVPSRFSGSGSGTDFTLTISSLQPEDFATYYCQQSYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 226 aa, Artificial Sequence
  Antibody 324 heavy chain
QVQLVQSGAEVKKPGASVKVSCKASGGTFNNYGISWVRQAPGQGLEWMGWMNPNSGNTGYAQKFQGRVTMTRDTSTSTVYMELSSLRSEDTAVYYCARVGDYGDYIVSPFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 59 - protein, 219 aa, Artificial Sequence
  Antibody 236 light chain
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPPTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 224 aa, Artificial Sequence
  Antibody 236 heavy chain
QVQLVQSGAEVKKPGASVKVSCKASGGTFTSYGINWVRQAPGQGLEWMGWMNPNSGNTGYAQKFQGRVTMTRDTSTSTVYMELSSLRSEDTAVYYCASRGIQLLPRGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 61 - protein, 214 aa, Artificial Sequence
  Antibody 52 light chain
DIQMTQSPSSLSASVGDRVTITCRASQGISNNLNWYQQKPGKAPKLLIYAASSLESGVPSRFSGSGSGTDFTLTISSLQPEDFATYYCQQGNGFPLTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 214 aa, Artificial Sequence
  Antibody 52 light chain N92T
DIQMTQSPSSLSASVGDRVTITCRASQGISNNLNWYQQKPGKAPKLLIYAASSLESGVPSRFSGSGSGTDFTLTISSLQPEDFATYYCQQGTGFPLTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 220 aa, Artificial Sequence
  Antibody 52 heavy chain
QVQLVQSGAEVKKPGSSVKVSCKASGYTFTSYGISWVRQAPGQGLEWMGGIIPMFGTTNYAQKFQGRVTITADKSTSTAYMELSSLRSEDTAVYYCARDRGDTIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 64 - protein, 220 aa, Artificial Sequence
  Antibody 80 light chain
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 227 aa, Artificial Sequence
  Antibody 80 heavy chain
QVQLVQSGAEVKKPGSSVKVSCKASGGTFNRYAFSWVRQAPGQGLEWMGGIIPIFGTANYAQKFQGRVTITADESTSTAYMELSSLRSEDTAVYYCARSTRELPEVVDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 66 - protein, 85 aa, Artificial Sequence
  Cferritin
GKTPDAMKAAMALEKKLNQALLDLHALGSARTDPHLCDFLETHFLDEEVKLIKKMGDHLTNLHRLGGPEAGLGEYLFERLTLKHD

SEQ ID NO: 67 - protein, 26 aa, Artificial Sequence
  Linker
GGSGGSGGSGGSGGGSGGSGGSGGSG

SEQ ID NO: 68 - protein, 116 aa, Artificial Sequence
  Polypeptide
QVQLVQSGAEVKKPGASVKVSCKASGYTFTSYGISWVRQAPGQGLEWMGWISAYNGNTNYAQKLQGRVTMTRDTSTSTVYMELSSLRSEDTAVYYCARDIGPIDYWGQGTLVTVSS

SEQ ID NO: 69 - protein, 123 aa, Artificial Sequence
  Polypeptide
EVQLLESGGGLVQPGGSLRLSCAASGFTFSNYGMHWVRQAPGKGLEWVSGISSAGSITNYADSVKGRFTISRDNSKNTLYLQMNSLRAEDTAVYYCAGNHAGTTVTSEYFQHWGQGTLVTVSS

SEQ ID NO: 70 - protein, 122 aa, Artificial Sequence
  Polypeptide
QVQLVQSGAEVKKPGASVKVSCKASGYTFTDYHMHWVRQAPGQGLEWMGWINPNSGGTNYAQKFQGRVTMTRDTSTSTVYMELSSLRSEDTAVYYCARDISSWYEITKFDPWGQGTLVTVSS

SEQ ID NO: 71 - protein, 123 aa, Artificial Sequence
  Polypeptide
QVQLVQSGAEVKKPGASVKVSCKASGYIFSRYAIHWVRQAPGQGLEWMGWMNPISGNTDYAPNFQGRVTMTRDTSTSTVYMELSSLRSEDTAVYYCAKDGSQLAYLVEYFQHWGQGTLVTVSS

SEQ ID NO: 72 - protein, 122 aa, Artificial Sequence
  Polypeptide
QVQLVQSGAEVKKPGASVKVSCKASGYTFTHYYMHWVRQAPGQGLEWMGIINPSSSSASYSQKFQGRVTMTRDTSTSTVYMELSSLRSEDTAVYYCARDGRYGSGSYPFDYWGQGTLVTVSS

SEQ ID NO: 73 - protein, 121 aa, Artificial Sequence
  Polypeptide
QVQLVQSGAEVKKPGASVKVSCKASGYTFTGHDMHWVRQAPGQGLEWMGIINPSGGSTSYAQKFQGRVTMTRDTSTSTVYMELSSLRSEDTAVYYCARANSLRYYYGMDVWGQGTMVTVSS

SEQ ID NO: 74 - protein, 117 aa, Artificial Sequence
  Polypeptide
QVQLVQSGAEVKKPGSSVKVSCKASGYTFTSYDINWVRQAPGQGLEWMGAIMPMFGTANYAQKFQGRVTITADESTSTAYMELSSLRSEDTAVYYCARGSSGYYYGWGQGTLVTVSS

SEQ ID NO: 75 - protein, 121 aa, Artificial Sequence
  Polypeptide
QVQLVQSGAEVKKPGSSVKVSCKASGGTFSSYAISWVRQAPGQGLEWMGWINPNSGGANYAQKFQGRVTITADESTSTAYMELSSLRSEDTAVYYCSTYYYDSSGYSTDYWGQGTLVTVSS

SEQ ID NO: 76 - protein, 122 aa, Artificial Sequence
  Polypeptide
QVQLVQSGAEVKKPGASVKVSCKASGYTFTGYYMHWVRQAPGQGLEWMGWINPLNGGTNFAPKFQGRVTMTRDTSTSTVYMELSSLRSEDTAVYYCARDPGGSYSNDAFDIWGQGTLVTVSS

SEQ ID NO: 77 - protein, 124 aa, Artificial Sequence
  Polypeptide
QVQLVQSGAEVKKPGSSVKVSCKASGYTFTSYAMHWVRQAPGQGLEWMGRISPRSGGTKYAQRFQGRVTITADESTSTAYMELSSLRSEDTAVYYCAREAVAGTHPQAGDFDLWGRGTLVTVSS

SEQ ID NO: 78 - protein, 119 aa, Artificial Sequence
  Polypeptide
EVQLLESGGGLVQPGGSLRLSCAASGFTFSSSAMHWVRQAPGKGLEWVSAIGTGGDTYYADSVKGRFTISRDNSKNTLYLQMNSLRAEDTAVYYCAREGDGYNFYFDYWGQGTLVTVSS

SEQ ID NO: 79 - protein, 124 aa, Artificial Sequence
  Polypeptide
QVQLVQSGAEVKKPGASVKVSCKASGYTFTSYDINWVRQAPGQGLEWMGMIDPSGGSTSYAQKFQGRVTMTRDTSTSTVYMELSSLRSEDTAVYYCAKDFGGGTRYDYWYFDLWGRGTLVTVSS

SEQ ID NO: 80 - protein, 121 aa, Artificial Sequence
  Polypeptide
EVQLLESGGGLVQPGGSLRLSCAASGFPFSQHGMHWVRQAPGKGLEWVSAIDRSGSYIYYADSVKGRFTISRDNSKNTLYLQMNSLRAEDTAVYYCARDTYGGKVTYFDYWGQGTLVTVSS

SEQ ID NO: 81 - protein, 119 aa, Artificial Sequence
  Polypeptide
QVQLVQSGAEVKKPGASVKVSCKASGGTFSTYGISWVRQAPGQGLEWMGWISPNSGGTDLAQKFQGRVTMTRDTSTSTVYMELSSLRSEDTAVYYCASDPRDDIAGGYWGQGTLVTVSS

SEQ ID NO: 82 - protein, 122 aa, Artificial Sequence
  Polypeptide
QVQLVQSGAEVKKPGASVKVSCKASGGSFSTSAFYWVRQAPGQGLEWMGWINPYTGGTNYAQKFQGRVTMTRDTSTSTVYMELSSLRSEDTAVYYCARSRALYGSGSYFDYWGQGTLVTVSS

SEQ ID NO: 83 - protein, 117 aa, Artificial Sequence
  Polypeptide
EVQLLESGGGLVQPGRSLRLSCAASGFTFSSYAMSWVRQAPGKGLEWVSTIYSGGSTYYADSVKGRFTISRDNSKNTLYLQMNSLRAEDTAVYYCARGDSRDAFDIWGQGTMVTVSS

SEQ ID NO: 84 - protein, 123 aa, Artificial Sequence
  Polypeptide
QVQLVQSGAEVKKPGASVKVSCKASGGTFNNYGISWVRQAPGQGLEWMGWMNPNSGNTGYAQKFQGRVTMTRDTSTSTVYMELSSLRSEDTAVYYCARVGDYGDYIVSPFDLWGRGTLVTVSS

SEQ ID NO: 85 - protein, 121 aa, Artificial Sequence
  Polypeptide
QVQLVQSGAEVKKPGASVKVSCKASGGTFTSYGINWVRQAPGQGLEWMGWMNPNSGNTGYAQKFQGRVTMTRDTSTSTVYMELSSLRSEDTAVYYCASRGIQLLPRGMDVWGQGTTVTVSS

SEQ ID NO: 86 - protein, 117 aa, Artificial Sequence
  Polypeptide
QVQLVQSGAEVKKPGSSVKVSCKASGYTFTSYGISWVRQAPGQGLEWMGGIIPMFGTTNYAQKFQGRVTITADKSTSTAYMELSSLRSEDTAVYYCARDRGDTIDYWGQGTLVTVSS

SEQ ID NO: 87 - protein, 124 aa, Artificial Sequence
  Polypeptide
QVQLVQSGAEVKKPGSSVKVSCKASGGTFNRYAFSWVRQAPGQGLEWMGGIIPIFGTANYAQKFQGRVTITADESTSTAYMELSSLRSEDTAVYYCARSTRELPEVVDWYFDLWGRGTLVTVSS

SEQ ID NO: 88 - protein, 108 aa, Artificial Sequence
  Polypeptide
DIQMTQSPSSLSASVGDRVTITCRASQGISSYLAWYQQKPGKAPKLLIYDASNLQSGVPSRFSGSGSGTDFTLTISSLQPEDFATYYCQQANSFPSTFGQGTKVEIKR

SEQ ID NO: 89 - protein, 108 aa, Artificial Sequence
  Polypeptide
DIQMTQSPSSLSASVGDRVTITCRASQSISSWLAWYQQKPGKAPKLLIYDTSNLETGVPSRFSGSGSGTDFTLTISSLQPEDFATYYCQQSYTTPWTFGQGTRLEIKR

SEQ ID NO: 90 - protein, 109 aa, Artificial Sequence
  Polypeptide
EIVMTQSPATLSVSPGERATLSCKASQSVSGTYLAWYQQKPGQAPRLLIYGASTRATGIPARFSGSGSGTEFTLTISSLQSEDFAVYYCLQTHSYPPTFGQGTKVEIKR

SEQ ID NO: 91 - protein, 108 aa, Artificial Sequence
  Polypeptide
DIQMTQSPSSLSASVGDRVTITCRASQVITNNLAWYQQKPGKAPKLLIYDASTLETGVPSRFSGSGSGTDFTLTISSLQPEDFATYYCQQSYTFPYTFGQGTKVEIKR

SEQ ID NO: 92 - protein, 108 aa, Artificial Sequence
  Polypeptide
DIQMTQSPSSLSASVGDRVTITCRASQNISRYLNWYQQKPGKAPKLLIYDASNLETGVPSRFSGSGSGTDFTLTISSLQPEDFATYYCQQANGFPPTFGQGTKLEIKR

SEQ ID NO: 93 - protein, 108 aa, Artificial Sequence
  Polypeptide
DIQMTQSPSSLSASVGDRVTITCRASQSVSSWLAWYQQKPGKAPKLLIYAASSLQSGVPSRFSGSGSGTDFTLTISSLQPEDFATYYCQQGYTTPYTFGQGTKLEIKR

SEQ ID NO: 94 - protein, 113 aa, Artificial Sequence
  Polypeptide
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPATFGPGTKVDIKR

SEQ ID NO: 95 - protein, 113 aa, Artificial Sequence
  Polypeptide
DIVMTQSPLSLPVTPGEPASISCRSSQSLLHSNGYNYLDWYLQKPGQSPQLLIYAASSLQSGVPDRFSGSGSGTDFTLKISRVEAEDVGVYYCMQALQTPYTFGQGTKLEIKR

SEQ ID NO: 96 - protein, 108 aa, Artificial Sequence
  Polypeptide
DIQMTQSPSSLSASVGDRVTITCRASQSISRYLNWYQQKPGKAPKLLIYDASNLESGVPSRFSGSGSGTDFTLTISSLQPEDFATYYCQQANSFPLTFGGGTKVDIKR

SEQ ID NO: 97 - protein, 113 aa, Artificial Sequence
  Polypeptide
DIVMTQSPLSLPVTPGEPASISCRSSQSLLHSNGYNYLDWYLQKPGQSPQLLIYAASSLQSGVPDRFSGSGSGTDFTLKISRVEAEDVGVYYCQQYYSSPYTFGQGTKLEIKR

SEQ ID NO: 98 - protein, 109 aa, Artificial Sequence
  Polypeptide
DIQMTQSPSSLSASVGDRVTITCRASQGISSYLAWYQQKPGKAPKLLIYDASSLQIGVPSRFSGSGSGTDFTLTISSLQPEDFATYYCLQSYSTPPWTFGQGTKVEIKR

SEQ ID NO: 99 - protein, 109 aa, Artificial Sequence
  Polypeptide
EIVMTQSPATLSVSPGERATLSCRASQSVSSRYLAWYQQKPGQAPRLLIYGASTRATGIPARFSGSGSGTEFTLTISSLQSEDFAVYYCQQYYTTPRTFGQGTRLEIKR

SEQ ID NO: 100 - protein, 108 aa, Artificial Sequence
  Polypeptide
DIQMTQSPSSLSASVGDRVTITCRASQGISSHLAWYQQKPGKAPKLLIYDASNLETGVPSRFSGSGSGTDFTLTISSLQPEDFATYYCQQTYSTPWTFGQGTKVEIKR

SEQ ID NO: 101 - protein, 114 aa, Artificial Sequence
  Polypeptide
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPPTFGQGTKLEIKR

SEQ ID NO: 102 - protein, 108 aa, Artificial Sequence
  Polypeptide
DIQMTQSPSSLSASVGDRVTITCRASQVISNYLAWYQQKPGKAPKLLIYDASNLETGVPSRFSGSGSGTDFTLTISSLQPEDFATYYCQQSFSPPPTFGQGTRLEIKR

SEQ ID NO: 103 - protein, 108 aa, Artificial Sequence
  Polypeptide
DIQMTQSPSSLSASVGDRVTITCRASQSISSWLAWYQQKPGKAPKLLIYDASNLETGVPSRFSGSGSGTDFTLTISSLQPEDFATYYCQQSYSTPFTFGPGTKVDIKR

SEQ ID NO: 104 - protein, 108 aa, Artificial Sequence
  Polypeptide
DIQMTQSPSSLSASVGDRVTITCRASQSITTYLNWYQQKPGKAPKLLIYDASNLETGVPSRFSGSGSGTDFTLTISSLQPEDFATYYCQQSYSTPPTFGQGTKVEIKR

SEQ ID NO: 105 - protein, 113 aa, Artificial Sequence
  Polypeptide
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPPTFGQGTRLEIKR

SEQ ID NO: 106 - protein, 108 aa, Artificial Sequence
  Polypeptide
DIQMTQSPSSLSASVGDRVTITCRASQGISNNLNWYQQKPGKAPKLLIYAASSLESGVPSRFSGSGSGTDFTLTISSLQPEDFATYYCQQGNGFPLTFGPGTKVDIKR

SEQ ID NO: 107 - protein, 114 aa, Artificial Sequence
  Polypeptide
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSAPLTFGGGTKVEIKR

SEQ ID NO: 108 - protein, 227 aa, Artificial Sequence
  Fc Chain 1
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 109 - DNA, 30 bp, Artificial Sequence
  Primer
cagatcacccggtttcagacactgctggcc

SEQ ID NO: 110 - DNA, 27 bp, Artificial Sequence
  Primer
gatgccgatgggcagatccaccagggg

SEQ ID NO: 111 - DNA, 30 bp, Artificial Sequence
  Primer
cggtaccggctgttccggaagtccaatctg

SEQ ID NO: 112 - protein, 25 aa, Artificial Sequence
  Primer
GTAATTGTGTTGCCGCCGACTTTGG

SEQ ID NO: 113 - DNA, 31 bp, Artificial Sequence
  Primer
gtgggcagcaccccttgtaacggcgtggaag

SEQ ID NO: 114 - DNA, 26 bp, Artificial Sequence
  Primer
ctgatagatctcggtggagatgtccc

SEQ ID NO: 115 - DNA, 31 bp, Artificial Sequence
  Primer
gccgaaggcttcaactgctacttcccactgc

SEQ ID NO: 116 - DNA, 25 bp, Artificial Sequence
  Primer
gccgttacaaggggtgctgccggcc

SEQ ID NO: 117 - DNA, 33 bp, Artificial Sequence
  Primer
aagaacctggactccaaagtcggcggcaactac

SEQ ID NO: 118 - DNA, 28 bp, Artificial Sequence
  Primer
gctgttccaggcaatcacacagccggtg

(19) United States
(12) Patent Application Publication
(10) Pub. No.: US 2025/0263469 A1
(43) Pub. Date: Aug. 21, 2025

(54) POLYPEPTIDES TARGETING SARS-COV-2 AND RELATED COMPOSITIONS AND METHODS

(71) Applicant: The Hospital for Sick Children, Toronto (CA)

(72) Inventors: Jean-Philippe Julien, Toronto (CA); Edurne Rujas Diez, Vitoria (ES)

(21) Appl. No.: 18/030,944

(22) PCT Filed: Oct. 8, 2021
(86) PCT No.: PCT/CA2021/051426

§ 371 Date: Oct. 17, 2023

Related U.S. Application Data
(60) Provisional application No. 63/220,929, filed on Jul. 12, 2021.
(60) Provisional application No. 63/197,236, filed on Jun. 4, 2021.
(60) Provisional application No. 63/089,782, filed on Oct. 9, 2020.

(51) Int. Cl. C07K 16/10; C07K 14/435; C12N 15/86

(52) U.S. Cl. CPC C07K 16/1003; C07K 14/435; C12N 15/86; C07K 2317/31; C07K 2317/35; C07K 2317/55; C07K 2317/569; C07K 2317/622; C07K 2317/76; C07K 2317/92; C07K 2319/30; C07K 2319/735; C12N 2740/16043

(57) ABSTRACT

Provided herein is a fusion protein comprises a nanocage monomer linked to a SARS-CoV-2 binding moiety, wherein a plurality of the fusion proteins self-assemble to form a nanocage. Also provided is a tri-specific antibody construct targeting SARS-CoV-2. Also provided is a fusion polypeptide comprising (1) a fragment crystallizable (Fc) region linked to (2) a nanocage monomer or subunit thereof, wherein the Fc region comprises the I253A mutation, wherein numbering is according to the EU index.

### CROSS-REFERENCE TO RELATED APPLICATIONS

This patent application is a U.S. National Stage Application of International Patent Application No. PCT/CA2021/051426, filed Oct. 8, 2021, which claims priority to and the benefit of U.S. Provisional Patent Application No. 63/089,782, filed Oct. 9, 2020, U.S. Provisional Patent Application No. 63/197,236, filed Jun. 4, 2021, and U.S. Provisional Patent Application No. 63/220,929, filed Jul. 12, 2021, all of which are incorporated herein by reference in their entireties.

### SEQUENCE LISTING

The instant application contains a Sequence Listing which has been submitted electronically in ASCII format and is hereby incorporated by reference in its entirety. The ASCII copy, created on Sep. 19, 2023, is named RBT-001WOUS_SL.txt and is 199,342 bytes in size.

### FIELD

The present invention relates to polypeptides. In particular, the present invention relates to SARS-CoV-2-specific polypeptides and related constructs, compositions, and methods.

### BACKGROUND

Nanoparticles have contributed to advancements in various disciplines. Their use has the potential to confer targeted delivery and allows the engineering of ordered micro-arrays, slow release and caged micro-environments for catalytic processes.

For the fabrication of nanoparticles that contain sensitive and metastable proteins, protein self-assembly is an attractive method. Indeed, self-assembled nanoparticles form under physiological conditions through non-covalent interactions and reliably yield uniform and often symmetric nanocapsules or nanocages.

Severe acute respiratory syndrome coronavirus 2 (SARS-CoV-2) is the strain of coronavirus that causes coronavirus disease 2019 (COVID-19), the respiratory illness responsible for the COVID-19 pandemic.

A need exists for improved compositions and methods for treating and/or preventing SARS-CoV-2.

### SUMMARY OF THE INVENTION

In accordance with an aspect, there is provided a fusion protein comprising a nanocage monomer linked to a SARS-CoV-2 binding moiety, wherein a plurality of the fusion proteins self-assemble to form a nanocage.

In an aspect, the SARS-CoV-2 binding moiety targets the SARS-CoV-2 S glycoprotein.

In an aspect, the SARS-CoV-2 binding moiety decorates the interior and/or exterior surface, preferably the exterior surface, of the assembled nanocage.

In an aspect, the SARS-CoV-2 binding moiety comprises an antibody or fragment thereof.

In an aspect, the antibody or fragment thereof comprises a Fab fragment.

In an aspect, the antibody or fragment thereof comprises a scFab fragment, a scFv fragment, a sdAb fragment, a VHH domains or a combination thereof.

In an aspect, the antibody or fragment thereof comprises a heavy and/or light chain of a Fab fragment.

In an aspect, the SARS-CoV-2 binding moiety comprises single chain variable domain VHH-72, BD23 and/or 4A8.

In an aspect, the SARS-CoV-2 binding moiety comprises an mAb listed in Table 4.

In an aspect, the SARS-CoV-2 binding moiety comprises mAb 298, 324, 46, 80, 52, 82, or 236 from Table 4.

In an aspect, the SARS-CoV-2 binding moiety is linked at the N- or C-terminus of the nanocage monomer, or wherein there is a first SARS-CoV-2 binding moiety linked at the N-terminus and a second SARS-CoV-2 binding moiety linked at the C-terminus of the nanocage monomer, wherein the first and second SARS-CoV-2 binding moieties are the same or different.

In an aspect, the nanocage monomer comprises a first nanocage monomer subunit linked to the SARS-CoV-2 binding moiety; wherein the first nanocage monomer subunit self-assembles with a second nanocage monomer subunit to form the nanocage monomer.

In an aspect, the SARS-CoV-2 binding moiety is linked at the N- or C-terminus of the first nanocage monomer, or wherein there is a first SARS-CoV-2 binding moiety linked at the N-terminus and a second SARS-CoV-2 binding moiety linked at the C-terminus of the first nanocage monomer subunit, wherein the first and second SARS-CoV-2 binding moieties are the same or different.

In an aspect, the fusion protein is provided in combination with the second nanocage monomer subunit.

In an aspect, the second nanocage monomer subunit is linked to a bioactive moiety.

In an aspect, the bioactive moiety comprises an Fc fragment.

In an aspect, the Fc fragment is an IgG1 Fc fragment.

In an aspect, the Fc fragment comprises one or more mutations, such as LS, YTE, LALA, I253A, and/or LALAP, that modulate the half-life of the fusion protein from, for example, minutes or hours to several days, weeks, or months.

In an aspect, the Fc fragment is an scFc fragment.

In an aspect, from about 3 to about 100 nanocage monomers, such as 24, 32, or 60 monomers, or from about 4 to about 200 nanocage monomer subunits, such as 4, 6, 8, 10, 12, 14, 18, 20, 22, 24, 26, 28, 30, 32, 34, 36, 38, 40, 42, 44, 46, 48, 50, or more, optionally in combination with one or more whole nanocage monomers, self-assemble to form a nanocage.

In an aspect, the nanocage monomer is selected from ferritin, apoferritin, encapsulin, SOR, lumazine synthase, pyruvate dehydrogenase, carboxysome, vault proteins, GroEL, heat shock protein, E2P, MS2 coat protein, fragments thereof, and variants thereof.

In an aspect, the nanocage monomer is apoferritin, optionally human apoferritin.

In an aspect, the first and second nanocage monomer subunits interchangeably comprise the “N” and “C” regions of apoferritin.

In an aspect, the “N” region of apoferritin comprises or consists of a sequence at least 70% (such as at least 75%, 80%, 85%, 90%, 95%, 96%, 97%, 98%, 99%, or 100%) identical to:
MSSQIRQNYSTDVEAAVNSLVNLYLQASYTYLSLGFYFDRDDVALEGVSHFFRELA EEKREGYERLLKMQNQRGGRALFQDIKKPAEDEW.

In an aspect, the “C” region of apoferritin comprises or consists of a sequence at least 70% (such as at least 75%, 80%, 85%, 90%, 95%, 96%, 97%, 98%, 99%, or 100%) identical to:
GKTPDAMKAAMALEKKLNQALLDLHALGSARTDPHLCDFLETHFLDEEVKLIKKMG DHLTNLHRLGGPEAGLGEYLFERLTLRHD

or
GKTPDAMKAAMALEKKLNQALLDLHALGSARTDPHLCDFLETHFLDEEVKLIKKMG DHLTNLHRLGGPEAGLGEYLFERLTLKHD.

In an aspect, the fusion protein further comprises a linker between the nanocage monomer subunit and the bioactive moiety.

In an aspect, the linker is flexible or rigid and comprises from about 1 to about 30 amino acid residues, such as from about 8 to about 16 amino acid residues.

In an aspect, the linker comprises a GGS repeat, such as 1, 2, 3, 4, or more GGS repeats.

In an aspect, the linker comprises or consists of a sequence at least 70% (such as at least 75%, 80%, 85%, 90%, 95%, 96%, 97%, 98%, 99%, or 100%) identical to:
GGGGSGGGGSGGGGSGGGGSGGGGSGG.

In an aspect, the fusion protein further comprises a C-terminal linker.

In an aspect, a C-terminal linker comprises or consists of a sequence at least 70% (such as at least 75%, 80%, 85%, 90%, 95%, 96%, 97%, 98%, 99%, or 100%) identical to:
GGSGGSGGSGGSGGGSGGSGGSGGSG In accordance with an aspect, there is provided a nanocage comprising at least one fusion protein described herein and at least one second nanocage monomer subunit that self-assembles with the fusion protein to form a nanocage monomer.

In an aspect, each nanocage monomer comprises the fusion protein described herein.

In an aspect, from about 20% to about 80% of the nanocage monomers comprise the fusion protein described herein.

In an aspect, the nanocage comprises at least 2, 3, 4, 5, 6, 7, 8, 9, or 10 different SARS-CoV-2 binding moieties, such as 3 different SARS-CoV-2 binding moieties.

In an aspect, the nanocage is multivalent and/or multispecific.

In an aspect, the nanocage comprises one of more mAbs from Table 4.

In an aspect, the nanocage comprises 3 mAbs from Table 4.

In an aspect, the nanocage comprises mAbs 298, 324, 46, 52, 80, 82 and/or 236 from Table 4.

In an aspect, the nanocage comprises a 4:2:1:1: ratio of scFab1-human apoferritin:scFc-human N-Ferritin:scFab2-C-Ferritin:scFab3-C-Ferritin.

In an aspect, the nanocage carries a cargo molecule, such as a pharmaceutical agent, a diagnostic agent, and/or an imaging agent.

In an aspect, the cargo molecule is not fused to the fusion protein and is contained in the nanocage internally.

In an aspect, the cargo molecule is a protein and is fused to the fusion protein such that the cargo molecule is contained in the nanocage internally.

In an aspect, the cargo molecule is a fluorescent protein, such as GFP, EGFP, Ametrine, and/or a flavin-based fluorescent protein, such as a LOV-protein, such as iLOV.

In accordance with an aspect, there is provided a tri-specific antibody construct targeting SARS-CoV-2.

In accordance with an aspect, there is provided a SARS-CoV-2 therapeutic or prophylactic composition comprising the nanocage or the antibody described herein.

In accordance with an aspect, there is provided a nucleic acid molecule encoding the fusion protein described herein.

In accordance with an aspect, there is provided a vector comprising the nucleic acid molecule described herein.

In accordance with an aspect, there is provided a host cell comprising the vector described herein and producing the fusion protein described herein.

In accordance with an aspect, there is provided a method for treating and/or preventing SARS-CoV-2, the method comprising administering the nanocage or the antibody or the composition described herein.

In accordance with an aspect, there is provided a use of the nanocage or the antibody or the composition described herein for treating and/or preventing SARS-CoV-2.

In accordance with an aspect, there is provided the nanocage or the antibody or the composition described herein for use in treating and/or preventing SARS-CoV-2.

In accordance with an aspect, there is provided a polypeptide comprising an amino acid sequence having at least 70% identity to any sequence listed in the following Table:

| | |
|---|---|
| QVQLVQSGAEVKKPGASVKVSCKASGYT | DIQMTQSPSSLSASVGDRVTITCRASQGI |
| FTSYGISWVRQAPGQGLEWMGWISAYN | SSYLAWYQQKPGKAPKLLIYDASNLQSGV |
| GNTNYAQKLQGRVTMTRDTSTSTVYMEL | PSRFSGSGSGTDFTLTISSLQPEDFATYY |
| SSLRSEDTAVYYCARDIGPIDYWGQGTLV | CQQANSFPSTFGQGTKVEIKR |
| TVSS | |
| EVQLLESGGGLVQPGGSLRLSCAASGFT | DIQMTQSPSSLSASVGDRVTITCRASQSIS |
| FSNYGMHWVRQAPGKGLEWVSGISSAG | SWLAWYQQKPGKAPKLLIYDTSNLETGVP |
| SITNYADSVKGRFTISRDNSKNTLYLQMN | SRFSGSGSGTDFTLTISSLQPEDFATYYC |
| SLRAEDTAVYYCAGNHAGTTVTSEYFQH | QQSYTTPWTFGQGTRLEIKR |
| WGQGTLVTVSS | |
| QVQLVQSGAEVKKPGASVKVSCKASGYT | EIVMTQSPATLSVSPGERATLSCKASQSV |
| FTDYHMHWVRQAPGQGLEWMGWINPNS | SGTYLAWYQQKPGQAPRLLIYGASTRAT |
| GGTNYAQKFQGRVTMTRDTSTSTVYMEL | GIPARFSGSGSGTEFTLTISSLQSEDFAVY |
| SSLRSEDTAVYYCARDISSWYEITKFDPW | YCLQTHSYPPTFGQGTKVEIKR |
| GQGTLVTVSS | |
| QVQLVQSGAEVKKPGASVKVSCKASGYIF | DIQMTQSPSSLSASVGDRVTITCRASQVIT |
| SRYAIHWVRQAPGQGLEWMGWMNPISG | NNLAWYQQKPGKAPKLLIYDASTLETGVP |
| NTDYAPNFQGRVTMTRDTSTSTVYMELS | SRFSGSGSGTDFTLTISSLQPEDFATYYC |
| SLRSEDTAVYYCAKDGSQLAYLVEYFQH | QQSYTFPYTFGQGTKVEIKR |
| WGQGTLVTVSS | |
| QVQLVQSGAEVKKPGASVKVSCKASGYT | DIQMTQSPSSLSASVGDRVTITCRASQNIS |
| FTHYYMHWVRQAPGQGLEWMGIINPSSS | RYLNWYQQKPGKAPKLLIYDASNLETGVP |
| SASYSQKFQGRVTMTRDTSTSTVYMELS | SRFSGSGSGTDFTLTISSLQPEDFATYYC |
| SLRSEDTAVYYCARDGRYGSGSYPFDYW | QQANGFPPTFGQGTKLEIKR |
| GQGTLVTVSS | |
| QVQLVQSGAEVKKPGASVKVSCKASGYT | DIQMTQSPSSLSASVGDRVTITCRASQSV |
| FTGHDMHWVRQAPGQGLEWMGIINPSG | SSWLAWYQQKPGKAPKLLIYAASSLQSG |
| GSTSYAQKFQGRVTMTRDTSTSTVYMEL | VPSRFSGSGSGTDFTLTISSLQPEDFATY |
| SSLRSEDTAVYYCARANSLRYYYGMDVW | YCQQGYTTPYTFGQGTKLEIKR |
| GQGTMVTVSS | |
| QVQLVQSGAEVKKPGSSVKVSCKASGYT | DIVMTQSPLSLPVTPGEPASISCRSSQSLL |
| FTSYDINWVRQAPGQGLEWMGAIMPMFG | HSNGYNYLDWYLQKPGQSPQLLIYLGSN |
| TANYAQKFQGRVTITADESTSTAYMELSS | RASGVPDRFSGSGSGTDFTLKISRVEAED |
| LRSEDTAVYYCARGSSGYYYGWGQGTLV | VGVYYCMQALQTPATFGPGTKVDIKR |
| TVSS | |
| QVQLVQSGAEVKKPGSSVKVSCKASGGT | DIVMTQSPLSLPVTPGEPASISCRSSQSLL |
| FSSYAISWVRQAPGQGLEWMGWINPNS | HSNGYNYLDWYLQKPGQSPQLLIYAASSL |
| GGANYAQKFQGRVTITADESTSTAYMELS | QSGVPDRFSGSGSGTDFTLKISRVEAEDV |
| SLRSEDTAVYYCSTYYYDSSGYSTDYWG | GVYYCMQALQTPYTFGQGTKLEIKR |
| QGTLVTVSS | |
| QVQLVQSGAEVKKPGASVKVSCKASGYT | DIQMTQSPSSLSASVGDRVTITCRASQSIS |
| FTGYYMHWVRQAPGQGLEWMGWINPLN | RYLNWYQQKPGKAPKLLIYDASNLESGVP |
| GGTNFAPKFQGRVTMTRDTSTSTVYMEL | SRFSGSGSGTDFTLTISSLQPEDFATYYC |
| SSLRSEDTAVYYCARDPGGSYSNDAFDI | QQANSFPLTFGGGTKVDIKR |
| WGQGTLVTVSS | |
| QVQLVQSGAEVKKPGSSVKVSCKASGYT | DIVMTQSPLSLPVTPGEPASISCRSSQSLL |
| FTSYAMHWVRQAPGQGLEWMGRISPRS | HSNGYNYLDWYLQKPGQSPQLLIYAASSL |
| GGTKYAQRFQGRVTITADESTSTAYMELS | QSGVPDRFSGSGSGTDFTLKISRVEAEDV |
| SLRSEDTAVYYCAREAVAGTHPQAGDFD | GVYYCQQYYSSPYTFGQGTKLEIKR |
| LWGRGTLVTVSS | |
| EVQLLESGGGLVQPGGSLRLSCAASGFT | DIQMTQSPSSLSASVGDRVTITCRASQGI |
| FSSSAMHWVRQAPGKGLEWVSAIGTGG | SSYLAWYQQKPGKAPKLLIYDASSLQIGV |
| DTYYADSVKGRFTISRDNSKNTLYLQMNS | PSRFSGSGSGTDFTLTISSLQPEDFATYY |
| LRAEDTAVYYCAREGDGYNFYFDYWGQ | CLQSYSTPPWTFGQGTKVEIKR |
| GTLVTVSS | |
| QVQLVQSGAEVKKPGASVKVSCKASGYT | EIVMTQSPATLSVSPGERATLSCRASQSV |
| FTSYDINWVRQAPGQGLEWMGMIDPSG | SSRYLAWYQQKPGQAPRLLIYGASTRAT |
| GSTSYAQKFQGRVTMTRDTSTSTVYMEL | GIPARFSGSGSGTEFTLTISSLQSEDFAVY |
| SSLRSEDTAVYYCAKDFGGGTRYDYWYF | YCQQYYTTPRTFGQGTRLEIKR |
| DLWGRGTLVTVSS | |
| EVQLLESGGGLVQPGGSLRLSCAASGFP | DIQMTQSPSSLSASVGDRVTITCRASQGI |
| FSQHGMHWVRQAPGKGLEWVSAIDRSG | SSHLAWYQQKPGKAPKLLIYDASNLETGV |
| SYIYYADSVKGRFTISRDNSKNTLYLQMN | PSRFSGSGSGTDFTLTISSLQPEDFATYY |
| SLRAEDTAVYYCARDTYGGKVTYFDYWG | CQQTYSTPWTFGQGTKVEIKR |
| QGTLVTVSS | |
| QVQLVQSGAEVKKPGASVKVSCKASGGT | DIVMTQSPDSLAVSLGERATINCKSSQSV |
| FSTYGISWVRQAPGQGLEWMGWISPNS | LYSSNNKNYLAWYQQKPGQPPKLLIYWA |
| GGTDLAQKFQGRVTMTRDTSTSTVYMEL | STRESGVPDRFSGSGSGTDFTLTISSLQA |
| SSLRSEDTAVYYCASDPRDDIAGGYWGQ | EDVAVYYCQQYYSTPPTFGQGTKLEIKR |
| GTLVTVSS | |
| QVQLVQSGAEVKKPGASVKVSCKASGGS | DIQMTQSPSSLSASVGDRVTITCRASQVIS |
| FSTSAFYWVRQAPGQGLEWMGWINPYT | NYLAWYQQKPGKAPKLLIYDASNLETGVP |
| GGTNYAQKFQGRVTMTRDTSTSTVYMEL | SRFSGSGSGTDFTLTISSLQPEDFATYYC |
| SSLRSEDTAVYYCARSRALYGSGSYFDY | QQSFSPPPTFGQGTRLEIKR |
| WGQGTLVTVSS | |
| EVQLLESGGGLVQPGRSLRLSCAASGFT | DIQMTQSPSSLSASVGDRVTITCRASQSIS |
| FSSYAMSWVRQAPGKGLEWVSTIYSGGS | SWLAWYQQKPGKAPKLLIYDASNLETGV |
| TYYADSVKGRFTISRDNSKNTLYLQMNSL | PSRFSGSGSGTDFTLTISSLQPEDFATYY |
| RAEDTAVYYCARGDSRDAFDIWGQGTMV | CQQSYSTPFTFGPGTKVDIKR |
| TVSS | |
| QVQLVQSGAEVKKPGASVKVSCKASGGT | DIQMTQSPSSLSASVGDRVTITCRASQSIT |
| FNNYGISWVRQAPGQGLEWMGWMNPN | TYLNWYQQKPGKAPKLLIYDASNLETGVP |
| SGNTGYAQKFQGRVTMTRDTSTSTVYME | SRFSGSGSGTDFTLTISSLQPEDFATYYC |
| LSSLRSEDTAVYYCARVGDYGDYIVSPFD | QQSYSTPPTFGQGTKVEIKR |
| LWGRGTLVTVSS | |
| QVQLVQSGAEVKKPGASVKVSCKASGGT | DIVMTQSPLSLPVTPGEPASISCRSSQSLL |
| FTSYGINWVRQAPGQGLEWMGWMNPNS | HSNGYNYLDWYLQKPGQSPQLLIYLGSN |
| GNTGYAQKFQGRVTMTRDTSTSTVYMEL | RASGVPDRFSGSGSGTDFTLKISRVEAED |
| SSLRSEDTAVYYCASRGIQLLPRGMDVW | VGVYYCMQALQTPPTFGQGTRLEIKR |
| GQGTTVTVSS | |
| QVQLVQSGAEVKKPGSSVKVSCKASGYT | DIQMTQSPSSLSASVGDRVTITCRASQGI |
| FTSYGISWVRQAPGQGLEWMGGIIPMFG | SNNLNWYQQKPGKAPKLLIYAASSLESGV |
| TTNYAQKFQGRVTITADKSTSTAYMELSS | PSRFSGSGSGTDFTLTISSLQPEDFATYY |
| LRSEDTAVYYCARDRGDTIDYWGQGTLV | CQQGNGFPLTFGPGTKVDIKR |
| TVSS | |
| QVQLVQSGAEVKKPGSSVKVSCKASGGT | DIVMTQSPDSLAVSLGERATINCKSSQSV |
| FNRYAFSWVRQAPGQGLEWMGGIIPIFGT | LYSSNNKNYLAWYQQKPGQPPKLLIYWA |
| ANYAQKFQGRVTITADESTSTAYMELSSL | STRESGVPDRFSGSGSGTDFTLTISSLQA |
| RSEDTAVYYCARSTRELPEVVDWYFDLW | EDVAVYYCQQYYSAPLTFGGGTKVEIKR |
| GRGTLVTVSS | |

or a functional fragment thereof.

In an aspect, the polypeptide comprises at least 75%, 80%, 85%, 90%, 95%, 96%, 97%, 98%, 99%, or 100% sequence identity to the listed sequence.

In an aspect, the polypeptide consists of at least 75%, 80%, 85%, 90%, 95%, 96%, 97%, 98%, 99%, or 100% sequence identity to the listed sequence.

In accordance with an aspect, there is provided an antibody or fragment thereof comprising the polypeptide described herein.

In accordance with an aspect, there is provided a fusion polypeptide comprising (1) a fragment crystallizable (Fc) region linked to (2) a nanocage monomer or subunit thereof, wherein the Fc region comprises the I253A mutation, wherein numbering is according to the EU index.

In an aspect, the Fc region further comprises the LALAP (L234A/L235A/P329G) mutations, wherein numbering is according to the EU index.

In an aspect, the Fc region is an IgG1 Fc region.

In an aspect, the nanocage monomer is a ferritin monomer.

In an aspect, the ferritin monomer is a ferritin light chain.

In an aspect, the ferritin light chain is a human ferritin light chain.

In an aspect, the Fc region is linked via an amino acid linker to the nanocage monomer or subunit thereof.

In an aspect, the Fc region is linked to the N-terminus of the nanocage monomer or subunit thereof.

In an aspect, the Fc region is a single chain Fc (scFc).

In an aspect, the Fc region is an Fc monomer.

In accordance with an aspect, there is provided a self-assembled polypeptide complex comprising:
(a) a plurality of first fusion polypeptides, each first fusion polypeptide comprising (1) an Fc region linked to (2) a nanocage monomer or subunit thereof, and
(b) a plurality of second fusion polypeptides, each second fusion polypeptide comprising (1) a SARS-CoV-2-binding antibody fragment linked to (2) a nanocage monomer or subunit thereof.

In an aspect, the nanocage monomer is a ferritin monomer.

In an aspect, the nanocage monomer is a ferritin light chain.

In an aspect, the self-assembled polypeptide complex does not comprise any ferritin heavy chains or subunits of ferritin heavy chains.

In an aspect, the nanocage monomer is a human ferritin light chain.

In an aspect, the SARS-CoV-2-binding antibody fragment binds to the receptor binding domain or the Spike protein of SARS-CoV-2.

In an aspect, the SARS-CoV-2-binding antibody fragment comprises a light chain variable domain and a heavy chain variable domain.

In an aspect, the SARS-CoV-2-binding antibody fragment comprises an Fab of an antibody that is capable of binding to SARS-CoV-2.

In an aspect, the SARS-CoV-2-binding antibody fragment comprises a VK domain and a VH domain.

In an aspect, the self-assembled polypeptide complex is characterized by a 1:1 ratio of first fusion polypeptides to second fusion polypeptides.

In an aspect, the Fc region is an IgG1 Fc region.

In an aspect, the Fc region is linked to the nanocage monomer or subunit thereof via an amino acid linker.

In an aspect, the Fc region is linked to the N-terminus of the nanocage monomer or subunit thereof.

In an aspect, the self-assembled polypeptide complex comprises at total of least 24 fusion polypeptides.

In an aspect, the self-assembled polypeptide complex comprises a total of at least 32 fusion polypeptides.

In an aspect, the self-assembled polypeptide complex has a total of about 32 fusion polypeptides.

In accordance with an aspect, there is provided a self-assembled polypeptide complex comprising:
(a) a plurality of first fusion polypeptides, each first fusion polypeptide comprising (1) an IgG1 Fc region linked to (2) a human ferritin monomer or subunit thereof, wherein the IgG1 Fc region comprises the LALAP (L234A/L235A/P329G) and I253A mutations, wherein numbering is according to the EU index, and
(b) a plurality of second fusion polypeptides, each second fusion polypeptide comprising (1) a Fab fragment of an antibody that is capable of binding to a SARS-CoV-2 protein, the Fab fragment being linked to (2) a human ferritin monomer or subunit thereof.

In an aspect:
(1) each first fusion polypeptide comprises a ferritin monomer subunit which is C-half-ferritin and each second fusion polypeptide comprises a ferritin monomer subunit which is N-half-ferritin; or
(2) each first fusion polypeptide comprises a ferritin monomer subunit which is N-half ferritin and each second fusion polypeptide comprises a ferritin monomer subunit which is C-half-ferritin.

In an aspect, the self-assembled polypeptide complex is characterized by a 1:1 ratio of first fusion polypeptides to second fusion polypeptides.

In an aspect, each first fusion polypeptide comprises a ferritin monomer subunit which is C-half-ferritin.

In an aspect, the IgG1 Fc region is linked to the C-half-ferritin via an amino acid linker.

In an aspect, the IgG1 Fc region is linked to the C-half-ferritin via the N-terminus of the C-half-ferritin.

In an aspect, each second fusion polypeptide comprises a ferritin monomer subunit which is N-half-ferritin.

In an aspect, the Fab fragment is linked to the N-half-ferritin via an amino acid linker.

In an aspect, the Fab fragment is linked to the N-half-ferritin via the N-terminus of the N-half-ferritin.

In an aspect, the self-assembled polypeptide complex further comprises a plurality of third fusion polypeptides, each third fusion polypeptide comprising (1) a human ferritin monomer linked to (2) a Fab fragment of an antibody that is capable of binding to a SARS-CoV-2 protein.

In an aspect, the self-assembled polypeptide complex is characterized by a 1:1:2 ratio of first fusion polypeptides to second fusion polypeptides to third fusion polypeptides.

In an aspect, the self-assembled polypeptide complex comprises at total of least 24 fusion polypeptides.

In an aspect, the self-assembled polypeptide complex comprises a total of at least 32 fusion polypeptides.

In an aspect, the self-assembled polypeptide complex has a total of 32 fusion polypeptides.

In an aspect, wherein the Fab fragment comprises a VK domain and a VH domain, wherein
(1) the VK domain has an amino acid sequence of SEQ ID NO: 11 and the VH domain has an amino acid sequence of SEQ ID NO:12;
(2) the VK domain has an amino acid sequence of SEQ ID NO:17 and the VH domain has an amino acid sequence of SEQ ID NO:18;
(3) the VK domain has an amino acid sequence of the VK within SEQ ID NO:25 and the VH domain has an amino acid sequence of the VH within SEQ ID NO:26;
(4) the VK domain has an amino acid sequence of the VK within SEQ ID NO:27 and the VH domain has an amino acid sequence of the VH within SEQ ID NO:28;
(5) the VK domain has an amino acid sequence of the VK within SEQ ID NO:29 and the VH domain has an amino acid sequence of the VH within SEQ ID NO:30;
(6) the VK domain has an amino acid sequence of the VK within SEQ ID NO:31 and the VH domain has an amino acid sequence of the VH within SEQ ID NO:32;
(7) the VK domain has an amino acid sequence of the VK within SEQ ID NO:33 and the VH domain has an amino acid sequence of the VH within SEQ ID NO:34;
(8) the VK domain has an amino acid sequence of the VK within SEQ ID NO:35 and the VH domain has an amino acid sequence of the VH within SEQ ID NO:36;
(9) the VK domain has an amino acid sequence of the VK within SEQ ID NO:37 and the VH domain has an amino acid sequence of the VH within SEQ ID NO:38;
(10) the VK domain has an amino acid sequence of the VK within SEQ ID NO:39 and the VH domain has an amino acid sequence of the VH within SEQ ID NO:40;
(11) the VK domain has an amino acid sequence of the VK within SEQ ID NO:41 and the VH domain has an amino acid sequence of the VH within SEQ ID NO:42;
(12) the VK domain has an amino acid sequence of the VK within SEQ ID NO:43 and the VH domain has an amino acid sequence of the VH within SEQ ID NO:44;
(13) the VK domain has an amino acid sequence of the VK within SEQ ID NO:45 and the VH domain has an amino acid sequence of the VH within SEQ ID NO:46;
(14) the VK domain has an amino acid sequence of the VK within SEQ ID NO:47 and the VH domain has an amino acid sequence of the VH within SEQ ID NO:48;
(15) the VK domain has an amino acid sequence of the VK within SEQ ID NO:49 and the VH domain has an amino acid sequence of the VH within SEQ ID NO:50;
(16) the VK domain has an amino acid sequence of the VK within SEQ ID NO:51 and the VH domain has an amino acid sequence of the VH within SEQ ID NO:52;
(17) the VK domain has an amino acid sequence of the VK within SEQ ID NO:53 and the VH domain has an amino acid sequence of the VH within SEQ ID NO:54;
(18) the VK domain has an amino acid sequence of the VK within SEQ ID NO:55 and the VH domain has an amino acid sequence of the VH within SEQ ID NO:56;
(19) the VK domain has an amino acid sequence of the VK within SEQ ID NO:57 and the VH domain has an amino acid sequence of the VH within SEQ ID NO:58;
(20) the VK domain has an amino acid sequence of the VK within SEQ ID NO:59 and the VH domain has an amino acid sequence of the VH within SEQ ID NO:60;
(21) the VK domain has an amino acid sequence of the VK within SEQ ID NO:61 or SEQ ID NO:62 and the VH domain has an amino acid sequence of the VH within SEQ ID NO:63; or
(22) the VK domain has an amino acid sequence of the VK within SEQ ID NO:64 and the VH domain has an amino acid sequence of the VH within SEQ ID NO:65.

In an aspect, the human ferritin monomer is human ferritin light chain.

In an aspect, the self-assembled polypeptide complex does not comprise any ferritin heavy chains or subunits of ferritin heavy chains.

In accordance with an aspect, there is provided a method of treating, ameliorating, or preventing a SARS-CoV-2-related condition, the method comprising administering to a subject a composition comprising the self-assembled polypeptide complex described herein.

In an aspect, the subject is a mammal.

In an aspect, the subject is human.

The novel features of the present invention will become apparent to those of skill in the art upon examination of the following detailed description of the invention. It should be understood, however, that the detailed description of the invention and the specific examples presented, while indicating certain aspects of the present invention, are provided for illustration purposes only because various changes and modifications within the spirit and scope of the invention will become apparent to those of skill in the art from the detailed description of the invention and claims that follow.

### BRIEF DESCRIPTION OF THE DRAWINGS

The present invention will be further understood from the following description with reference to the Figures, in which:

FIG. 1. Avidity enhances binding and neutralization of VHH against SARS-CoV-2. a Schematic representation of a monomeric VHH domain and its multimerization using a conventional Fc (dark red) scaffold or human apoferritin (gray). b Size exclusion chromatography and SDS-PAGE of apoferritin alone (gray) and VHH-72 apoferritin particles (gold). c Negative stain electron microscopy of VHH-72 apoferritin particles. (Scale bar 50 nm, representative of two independent experiments). d Comparison of the binding avidity (apparent K_{D}) of VHH-72 to SARS-CoV-2 S protein when displayed in a bivalent (dark red) or 24-mer (gold) format. Bars indicate the mean values of n=2 biologically independent experiments. Apparent K_{D} lower than 10⁻¹=M (dash line) is beyond the instrument detection limit. e Neutralization potency against SARS-CoV-2 PsV (color coding is as in (d)). One representative out of two biologically independent replicates with similar results is shown. Mean values ±SD of two technical replicates is represented in the plot. Median IC₅₀ values of the two biologically independent replicates are shown.

FIG. 2. Binding interfaces of Fabs 52 and 298 and the RBD. Interaction of Fab 298 (a) and 52 (b) with RBD (light and dark green for the core and RBM regions, respectively) is mediated by complementarity determining regions (CDR) heavy (H) 1 (yellow), H2 (orange), H3 (red), kappa light (K) 1 (light blue) and K3 (purple). Critical binding residues are shown as sticks (insets). H-bonds and salt bridges are depicted as black dashed lines. L and H chains of Fabs are shown in tan and white, respectively. c) Bottom and side views of ACE2 (left) and Fab 298 (right) bound to RBD. RBD side-chains that are part of the binding interface of the ACE2-RBD and Fab 298-RBD complexes are depicted in pink, while RBD side-chains unique to a given interface are shown in yellow. Surfaces of ACE2, variable regions of Fab 298 HC and Fab 298 KC are shown in white, grey and tan, respectively. The RBD is colored as in (a). d) Superposition of Fabs 46 (light pink) and 52 (dark pink) when bound to the RBD (green) reveals a distinct angle of approach for the two mAbs. Stereo-image of the composite omit map electron density contoured at 1.3 sigma at the interfaces of e) 298-RBD and f) 52-RBD.

FIG. 3. Bioavailability, biodistribution, and immunogenicity of a mouse surrogate Multabody. a Binding kinetics of WT and Fc-modified (LALAP mutation) MB to mouse FcγRI (left) and mouse FcRn at endosomal (middle) and physiological (right) pH in comparison to the parental IgG. Two-fold dilution series from 100 to 3 nM (IgG) and 10 to 0.3 nM (MB) were used. Red lines represent raw data; black lines represent global fits. b Five male C57BL/6 mice per group were used to assess the serum concentration of a surrogate mouse MB, a Fc-modified MB (LALAP mutation), and parental mouse IgGs (IgG1 and IgG2a subtypes) after subcutaneous administration of 5 mg/kg. c MB and IgG2a samples were labeled with Alexa-647 for visualization of their biodistribution post subcutaneous injection into three male BALB/c mice/group via live noninvasive 2D whole body imaging. 15 nm fluorescently-labeled gold nanoparticles (GNP), which have a similar Rh value as the Multabody are shown as a comparator. d Five male C57BL/6 mice per group were used to assess any anti-drug-antibody response induced by the mouse surrogate Multabody in comparison to parental IgG and a species-mismatched malaria PfCSP peptide fused to Helicobacter pylori ferritin (HpFerr). Mean values ±SD of n=5 mice is shown in (b) and (d).

FIG. 4. 3D biodistribution of a surrogate mouse Multabody is comparable to its parental IgG. The biodistribution of 15 nm gold nanoparticles (GNP), MB and IgG samples labeled with Alexa-647 were visualized post subcutaneous injection into BALB/c mice via live non-invasive 3D whole body imaging. a) Representative 3D rendered fluorescent image overlaid with CT scan from PBS injected control. b) Depiction of the localization of major mouse organs overlaid with CT scan. c) 3D rendered fluorescent images overlaid with CT scan at 1 h (1H), two days (D2), eight days (D8) and 11 days (D11) post subcutaneous injection of gold nanoparticles (top), MB (three middle panels) or IgG (bottom panel). Each 3D image set is displayed showing dorsal view overlaid with CT scan (right), as well as a selected frontal (top left), medial (middle), and transverse (bottom left) planes based on signal localization. 3D fluorescent images were mapped to a rainbow look-up Table (LUT), with color scale minimum set to background and maximum set to 50 pmol M⁻¹ cm⁻¹ (GNP) or 1000 pmol M⁻¹ cm⁻¹ (MB and IgG).

FIG. 5. Protein engineering to multimerize IgG-like particles against SARS-CoV-2. a Schematic representation of the human apoferritin split design. b Negative stain electron micrograph of the MB. (Scale bar 50 nm, representative of two independent experiments). c Hydrodynamic radius (Rh) of the MB. d Avidity effect on the binding (apparent Kc) of 4A8 (purple) and BD23 (gray) to the SARS-CoV-2 Spike. e Sensograms of BD23 IgG and MB with different Fc sequence variants binding to FcγRI (top row), FcRn at endosomal pH (middle row) and FcRn at physiological pH (bottom row). Red lines represent raw data whereas black lines represent global fits. f Neutralization of SARS-CoV-2 PsV by 4A8 and BD23 IgGs and MBs. Representative data of three biologically independent samples. The mean values ±SD for two technical replicates is shown in each neutralization plot. Median IC₅₀ values of the three biologically independent replicates are indicated.

FIG. 6. The Multabody enhances the potency of human mAbs from phage display. a Work flow for the identification of potent anti-SARS-CoV-2 neutralizers using the MB technology. Created with Biorender. b Comparison of neutralization potency between IgGs (cyan) and MBs (pink) that display the same human Fab sequences derived from phage display. c IC₅₀ values fold increase upon multimerization. d Apparent affinity (K_{D}), association (kₒₙ), and dissociation (k_{off}) rates of the most potent neutralizing MBs (pink) compared to their IgG counterparts (cyan) for binding the SARS-CoV-2 S protein. Three biological replicates and their mean are shown for IC₅₀ values in (b) and (c).

FIG. 7. Neutralization of SARS-CoV-2 RBD-targeting Multabodies and their parental IgGs. a) Representative neutralization titration curves of 20 antibodies against SARS-CoV-2 PsV when displayed as IgGs (black) and MBs (dark red). The mean IC₅₀ values of three biological replicates are displayed for comparison. The mean values ±SD for two technical replicates are shown in each neutralization plot. b) Neutralization profiles of selected IgGs and MBs against SARS-CoV-2 PsV targeting 293T-ACE2 (black) and HeLa-ACE2 (gray) target cells. The mean IC₅₀ value and individual IC₅₀ values of three and two biological replicates are shown for 293T-ACE2 and HeLa-ACE2 cells, respectively. c) Neutralization titration curves of three biological replicates (different shades of gray) against the authentic SARS-CoV-2/SB2-P4-PB strain. The mean IC₅₀ is indicated. Neutralization potencies of recombinant mAbs REGN10933 (red) and REGN10987 (blue) are included in (a) and (c) as benchmarks for comparison.

FIG. 8. Expression yields and homogeneity of SARS-CoV-2 RBD-targeting Multabodies. a) Yield (mg/L) of the seven most potent IgGs (white) and their respective MBs (dark red). Mean values ±SD for two biologically independent samples. b) Aggregation temperature (Tagg, ° C.) comparison as in (a). The solid line denotes the mean Tagg value of two biologically independent samples. c) SEC chromatograms of 298 IgG (top row, black) and 298 MB (bottom row, dark red) from three independent expressions and purifications. Prior to SEC, in both cases, the samples were purified using Protein A affinity chromatography. The arrows indicate the peak used to perform a PsV neutralization assay from each batch. IC₅₀ values (μg/mL) are noted. Mean values ±SD for two technical replicates are shown in each neutralization plot.

FIG. 9. Binding profiles of IgGs and MBs. Sensograms of IgGs and MBs binding to RBD (left) and S protein (right) of SARS-CoV-2 immobilized onto Ni-NTA biosensors. 2-fold dilution series from 125 to 4 nM (IgG), and 16 to 0.5 nM (MB) were used. Red lines represent raw data, whereas black lines represent global fits.

FIG. 10. Epitope delineation of the most potent mAb specificities. a Surface and cartoon representation of RBD (light green for the core and dark green for RBM) and ACE2⁶⁶ (light brown) binding. Heat map showing binding competition experiments. High signal responses (red) represent low competition while low signal responses (white) correspond to high competition. Epitope bins are highlighted by dashed-line boxes. b 15.0 Å filtered cryo-EM reconstruction of the Spike (gray) in complex with Fab 80 (yellow), 298 (orange), and 324 (red). The RBD and NTD are shown in green and blue, respectively. c Cryo-EM reconstruction of the Fab 46 (pink) and RBD (green) complex. A RBD⁶⁶ secondary structure cartoon is fitted into the partial density observed for the RBD. d Crystal structure of the ternary complex formed by Fab 52 (purple), Fab 298 (orange), and RBD (green). e Composite image depicting the side and top view of the unliganded (PDB 6XM4) and the antibody-bound SARS-CoV-2 spike with available PDB or EMD entries^{3,4,9,10,13,15,17,67,68,69,70,72}. Inset: close up view of antibodies targeting different antigenic sites on the RBD. The mAb with the lowest reported IC₅₀ value against SARS-CoV-2 PsV was selected as a representative antibody of the bin (highlighted in bold) and those antibodies with similar binding epitopes are listed in the same color below (color coding of Spike, NTD and RBD as in (b)). Individual protomers in the unliganded spike are shown in white, pink, and purple.

FIG. 11. Epitope binning. mAb binding competition experiments to His-tagged RBD as measured by biolayer interferometry (BLI). 50 μg/ml of mAb 1 was incubated for 3 min followed by incubation with 50 μg/ml of mAb 2 for 5 min.

FIG. 12. Cryo-EM analysis of the Fab-Spike and Fab-RBD complexes. Representative cryo-EM micrograph (scale bar 50 nm, top left), selected 2D class averages (top right), Fourier shell correlation curve from the final 3D non-uniform refinement (bottom left) and local resolution (A) plotted on the surface of the cryo-EM map (bottom right) are shown for the Fab 80-Spike complex (a), the Fab 298-Spike complex (b), the Fab 324-Spike complex (c), and the Fab 46-RBD complex (d).

FIG. 13. Multabodies overcome SARS-CoV-2 sequence diversity. a Cartoon representation of the RBD showing four naturally occurring mutations as spheres. The epitopes of mAbs 52 (light pink) and 298 (yellow) are shown as representative epitopes of each bin. b Affinity and c IC₅₀ fold-change comparison between WT and mutated RBD and PsV, respectively. d Neutralization potency of IgG (gray bars) vs MB (dark red bars) against SARS-CoV-2 PsV variants in comparison to WVT PsV. e Neutralization potency comparison of two IgG cocktails (three IgGs), monospecific MB cocktails (three MBs) and tri-specific MBs against WT SARS-CoV-2 PsV and variants. mAbs sensitive to one or more PsV variants (d) were selected to generate the cocktails and the tri-specific MBs. f Neutralization potency of the tri-specific 298-80-52 MB against SARS-CoV-2 B.1.351 PsV variant. g IC₅₀ values in PsV (y-axis) and replication competent SARS-CoV-2 virus (SB2-P4-PB: x-axis) demonstrating the ability of tri-specific MBs (red) to enhance potency across a wide range of mAb characteristics (blue and black). h IC₅₀ values fold increase upon multimerization. The mean of three biological replicates is shown in (b-h).

FIG. 14. MBs potently overcome SARS-CoV-2 sequence variability. a) Comparison of the neutralization potency of selected IgGs and MBs against WT PsV (dark red) and the more infectious D614G PsV (grey). b) Schematic representation of a tri-specific MB generated by combination of three Fab specificities and the Fc fragment using the MB split design. c) Cocktails and tri-specific MBs that combine the specificities of mAbs 298, 80 and 52, or 298, 324 and 46 were generated and tested against WT PsV. The mean values ±SD for two technical replicates is represented in each representative neutralization plot. Source data are provided as a Source Data file. d) Neutralization potency change of cocktails and tri-specific MBs against pseudotyped SARS-CoV-2 variants in comparison to WT PsV. PsV variants that were sensitive to individual antibodies within the cocktails were selected. The area within the dotted lines represents a 3-fold change in IC50 value. This threshold was established as the cut-off for increased sensitivity (up bars) or increased resistance (down bars). e) Neutralization titration curves showing three biological replicates of cocktails and tri-specific MBs against the authentic SARS-CoV-2/SB2-P4-PB strain. Mean IC50 values of three biologically independent replicates are shown.

FIG. 15. The N92T mutation did not have any effects in potency as both an IgG or as a monospecific MB in a WT pseudovirus neutralization assay.

FIG. 16. The 298-80-52 trispecific MB containing the N92T mutation in the VL of mAb 52 was screened in a P.1 PsV neutralization assay and the results confirmed that there was no loss in potency observed compared to the parental trispecific MB.

FIG. 17. An example trispecific MB, 298-80-52, was assessed for potency across the variants of concern (VOCs) in pseudovirus neutralization assays.

### DETAILED DESCRIPTION OF CERTAIN ASPECTS

### Definitions

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Although any methods and materials similar or equivalent to those described herein can be used in the practice for testing of the present invention, the typical materials and methods are described herein. In describing and claiming the present invention, the following terminology will be used.

It is also to be understood that the terminology used herein is for the purpose of describing particular aspects only, and is not intended to be limiting. Many patent applications, patents, and publications are referred to herein to assist in understanding the aspects described. Each of these references are incorporated herein by reference in their entirety.

In understanding the scope of the present application, the articles “a”, “an”, “the”, and “said” are intended to mean that there are one or more of the elements. Additionally, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

It will be understood that any aspects described as “comprising” certain components may also “consist of” or “consist essentially of,” wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Typically, a composition consisting essentially of a set of components will comprise less than 5% by weight, typically less than 3% by weight, more typically less than 1%, and even more typically less than 0.1% by weight of non-specified component(s).

It will be understood that any component defined herein as being included may be explicitly excluded from the claimed invention by way of proviso or negative limitation. For example, in some aspects the nanocages and/or fusion proteins described herein may exclude a ferritin heavy chain and/or may exclude an iron-binding component.

In addition, all ranges given herein include the end of the ranges and also any intermediate range points, whether explicitly stated or not.

Terms of degree such as “substantially”, “about” and “approximately” as used herein mean a reasonable amount of deviation of the modified term such that the end result is not significantly changed. These terms of degree should be construed as including a deviation of up to and including at least ±5% of the modified term if this deviation would not negate the meaning of the word it modifies. For example, the term “about” may encompass a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.”

The abbreviation, “e.g.” is derived from the Latin exempli gratia, and is used herein to indicate a non-limiting example. Thus, the abbreviation “e.g.” is synonymous with the terms “for example,” or “such as.” The word “or” is intended to include “and” unless the context clearly indicates otherwise.

The term “subject” as used herein refers to any member of the animal kingdom, typically a mammal. The term “mammal” refers to any animal classified as a mammal, including humans, other higher primates, domestic and farm animals, and zoo, sports, or pet animals, such as dogs, cats, cattle, horses, sheep, pigs, goats, rabbits, etc. Typically, the mammal is human.

The terms “protein nanoparticle,” “nanocage,” and “multabody” are used interchangeably herein and refer to a multi-subunit, protein-based polyhedron shaped structure. The subunits or nanocage monomers are each composed of proteins or polypeptides (for example a glycosylated polypeptide), and, optionally of single or multiple features of the following: nucleic acids, prosthetic groups, organic and inorganic compounds. Non-limiting examples of protein nanoparticles include ferritin nanoparticles (see, e.g., Zhang, Y. Int. J. Mol. Sci., 12:5406-5421, 2011, incorporated by reference herein), encapsulin nanoparticles (see, e.g., Sutter et al., Nature Struct, and Mol. Biol., 15:939-947, 2008, incorporated by reference herein), Sulfur Oxygenase Reductase (SOR) nanoparticles (see, e.g., Urich et al., Science, 311:996-1000, 2006, incorporated by reference herein), lumazine synthase nanoparticles (see, e.g., Zhang et al., J. Mol. Biol., 306: 1099-1114, 2001) or pyruvate dehydrogenase nanoparticles (see, e.g., Izard et al., PNAS 96: 1240-1245, 1999, incorporated by reference herein). Ferritin, apoferritin, encapsulin, SOR, lumazine synthase, and pyruvate dehydrogenase are monomeric proteins that self-assemble into a globular protein complexes that in some cases consists of 24, 60, 24, 60, and 60 protein subunits, respectively. Ferritin and apoferritin are generally referred to interchangeably herein and are understood to both be suitable for use in the fusion proteins, nanocages, and methods described herein. Carboxysome, vault proteins, GroEL, heat shock protein, E2P and MS2 coat protein also produce nanocages are contemplated for use herein. In addition, fully or partially synthetic self-assembling monomers are also contemplated for use herein.

It will be understood that each nanocage monomer may be divided into two or more subunits that will self-assemble into a functional nanocage monomer. For example, ferritin or apoferritin may be divided into an N- and C-subunit, e.g., an N- and C-subunit obtained by dividing full-length ferritin substantially in half, so that each subunit may be separately bound to a different SARS-CoV-2 binding moiety or bioactive moiety for subsequent self-assembly into a nanocage monomer and then a nanocage. Each subunit may, in aspects, bind a SARS-CoV-2 binding moiety and/or bioactive moiety at both termini, either the same or different. By “functional nanocage monomer” it is intended that the nanocage monomer is capable of self-assembly with other such monomers into a nanocage as described herein.

The terms “ferritin” and “apoferritin” are used interchangeably herein and generally refer to a polypeptide (e.g., a ferritin chain) that is capable of assembling into a ferritin complex which typically comprises 24 protein subunits. It will be understood that the ferritin can be from any species. Typically, the ferritin is a human ferritin. In some embodiments, the ferritin is a wild-type ferritin. For example, the ferritin may be a wild-type human ferritin. In some embodiments, a ferritin light chain is used as a nanocage monomer, and/or a subunit of a ferritin light chain is used as a nanocage monomer subunit. In some embodiments, assembled nanocages do not include any ferritin heavy chains or other ferritin components capable of binding to iron.

The term “multispecific,” as used herein, refers to the characteristic of having at least two binding sites at which at least two different binding partners, e.g., an antigen or receptor (e.g., Fc receptor), can bind. For example, a nanocage that comprises at least two Fab fragments, wherein each of the two Fab fragments binds to a different antigen, is “multispecific.” As an additional example, a nanocage that comprises an Fc fragment (which is capable of binding to an Fc receptor) and an Fab fragment (which is capable of binding to an antigen) is “multispecific.”

The term “multivalent,” as used herein, refers to the characteristic of having at least two binding sites at which a binding partner, e.g., an antigen or receptor (e.g., Fc receptor), can bind. The binding partners that can bind to the at least two binding sites may be the same or different.

The term “antibody”, also referred to in the art as “immunoglobulin” (Ig), used herein refers to a protein constructed from paired heavy and light polypeptide chains; various Ig isotypes exist, including IgA, IgD, IgE, IgG, such as IgG₁, IgG₂, IgG₃, and IgG₄, and IgM. It will be understood that the antibody may be from any species, including human, mouse, rat, monkey, llama, or shark. When an antibody is correctly folded, each chain folds into a number of distinct globular domains joined by more linear polypeptide sequences. For example, in the case of IgGs, the immunoglobulin light chain folds into a variable (V_{L}) and a constant (CL) domain, while the heavy chain folds into a variable (V_{H}) and three constant (C_{H}, C_{H2}, C_{H3}) domains. Interaction of the heavy and light chain variable domains (V_{H} and V_{L}) results in the formation of an antigen binding region (Fv). Each domain has a well-established structure familiar to those of skill in the art.

The light and heavy chain variable regions are responsible for binding the target antigen and can therefore show significant sequence diversity between antibodies. The constant regions show less sequence diversity, and are responsible for binding a number of natural proteins to elicit important immunological events. The variable region of an antibody contains the antigen binding determinants of the molecule, and thus determines the specificity of an antibody for its target antigen. The majority of sequence variability occurs in six hypervariable regions, three each per variable heavy and light chain; the hypervariable regions combine to form the antigen-binding site, and contribute to binding and recognition of an antigenic determinant. The specificity and affinity of an antibody for its antigen is determined by the structure of the hypervariable regions, as well as their size, shape and chemistry of the surface they present to the antigen.

An “antibody fragment” as referred to herein may include any suitable antigen-binding antibody fragment known in the art. The antibody fragment may be a naturally-occurring antibody fragment, or may be obtained by manipulation of a naturally-occurring antibody or by using recombinant methods. For example, an antibody fragment may include, but is not limited to a Fv, single-chain Fv (scFv; a molecule consisting of V_{L} and V_{H} connected with a peptide linker), Fc, single-chain Fc, Fab, single-chain Fab, F(ab′)₂, single domain antibody (sdAb; a fragment composed of a single V_{L} or V_{H}), and multivalent presentations of any of these.

By the term “synthetic antibody” as used herein, is meant an antibody which is generated using recombinant DNA technology. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “epitope” refers to an antigenic determinant. An epitope is the particular chemical groups or peptide sequences on a molecule that are antigenic, that is, that elicit a specific immune response. An antibody specifically binds a particular antigenic epitope, e.g., on a polypeptide. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, about 11, or about 8 to about 12 amino acids in a unique spatial conformation. Methods of determining spatial conformation of epitopes include, for example, x-ray crystallography and 2-dimensional nuclear magnetic resonance. See, e.g., “Epitope Mapping Protocols” in Methods in Molecular Biology, Vol. 66, Glenn E. Morris, Ed (1996).

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the aspects described herein include, but are not limited to, the use of partial nucleotide sequences of more than one gene and that these nucleotide sequences could be arranged in various combinations to elicit the desired immune response. Moreover, a skilled artisan will understand that an antigen need not be encoded by a “gene” at all. It is readily apparent that an antigen can be synthesized or can be derived from a biological sample. Such a biological sample can include, but is not limited to a tissue sample, a cell, or a biological fluid.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, typically, a human.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Parenteral” administration of composition includes, e.g., subcutaneous (s.c.), intravenous (i.v.), intramuscular (i.m.), or intrasternal injection, or infusion techniques. Also included are inhalation and intranasal administration.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The terms “therapeutically effective amount”, “effective amount” or “sufficient amount” mean a quantity sufficient, when administered to a subject, including a mammal, for example a human, to achieve a desired result, for example an amount effective to cause a protective immune response. Effective amounts of the compounds described herein may vary according to factors such as the molecule, age, sex, species, and weight of the subject. Dosage or treatment regimes may be adjusted to provide the optimum therapeutic response, as is understood by a skilled person. For example, administration of a therapeutically effective amount of the fusion proteins described herein is, in aspects, sufficient to treat and/or prevent COVID-19.

Moreover, a treatment regime of a subject with a therapeutically effective amount may consist of a single administration, or alternatively comprise a series of applications. The frequency and length of the treatment period depends on a variety of factors, such as the molecule, the age of the subject, the concentration of the agent, the responsiveness of the patient to the agent, or a combination thereof. It will also be appreciated that the effective dosage of the agent used for the treatment may increase or decrease over the course of a particular treatment regime. Changes in dosage may result and become apparent by standard diagnostic assays known in the art. The fusion proteins described herein may, in aspects, be administered before, during or after treatment with conventional therapies for the disease or disorder in question. For example, the fusion proteins described herein may find particular use in combination with conventional treatments for viral infections.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Administration “in combination with” one or more further therapeutic agents includes simultaneous (concurrent) and consecutive administration in any order.

The term “pharmaceutically acceptable” means that the compound or combination of compounds is compatible with the remaining ingredients of a formulation for pharmaceutical use, and that it is generally safe for administering to humans according to established governmental standards, including those promulgated by the United States Food and Drug Administration.

The term “pharmaceutically acceptable carrier” includes, but is not limited to solvents, dispersion media, coatings, antibacterial agents, antifungal agents, isotonic and/or absorption delaying agents and the like. The use of pharmaceutically acceptable carriers is well known.

“Variants” are biologically active fusion proteins, antibodies, or fragments thereof having an amino acid sequence that differs from a comparator sequence by virtue of an insertion, deletion, modification and/or substitution of one or more amino acid residues within the comparative sequence. Variants generally have less than 100% sequence identity with the comparative sequence. Ordinarily, however, a biologically active variant will have an amino acid sequence with at least about 70% amino acid sequence identity with the comparative sequence, such as at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity. The variants include peptide fragments of at least 10 amino acids that retain some level of the biological activity of the comparator sequence. Variants also include polypeptides wherein one or more amino acid residues are added at the N- or C-terminus of, or within, the comparative sequence. Variants also include polypeptides where a number of amino acid residues are deleted and/or optionally substituted by one or more amino acid residues. Variants also may be covalently modified, for example by substitution with a moiety other than a naturally occurring amino acid or by modifying an amino acid residue to produce a non-naturally occurring amino acid.

“Percent amino acid sequence identity” is defined herein as the percentage of amino acid residues in the candidate sequence that are identical with the residues in the sequence of interest, such as the polypeptides of the invention, after aligning the sequences and introducing gaps, if necessary, to achieve the maximum percent sequence identity, and not considering any conservative substitutions as part of the sequence identity. None of N-terminal, C-terminal, or internal extensions, deletions or insertions into the candidate sequence shall be construed as affecting sequence identity or homology. Methods and computer programs for the alignment are well known in the art, such as “BLAST”.

“Active” or “activity” for the purposes herein refers to a biological and/or an immunological activity of the fusion proteins described herein, wherein “biological” activity refers to a biological function (either inhibitory or stimulatory) caused by the fusion proteins.

The fusion proteins described herein may include modifications. Such modifications include, but are not limited to, conjugation to an effector molecule. Modifications further include, but are not limited to conjugation to detectable reporter moieties. Modifications that extend half-life (e.g., pegylation) are also included. Modifications for de-immunization are also included. Proteins and non-protein agents may be conjugated to the fusion proteins by methods that are known in the art. Conjugation methods include direct linkage, linkage via covalently attached linkers, and specific binding pair members (e.g., avidin-biotin). Such methods include, for example, that described by Greenfield et al., Cancer Research 50, 6600-6607 (1990), which is incorporated by reference herein and those described by Amon et al., Adv. Exp. Med. Biol. 303, 79-90 (1991) and by Kiseleva et al, Mol. Biol. (USSR)25, 508-514 (1991), both of which are incorporated by reference herein.

### Fusion Proteins

Described herein are fusion proteins. The fusion proteins comprise a nanocage monomer linked to a SARS-CoV-2 binding moiety. A plurality of the fusion proteins self-assemble to form a nanocage. In this way, the SARS-CoV-2 binding moiety may decorate the interior surface of the assembled nanocage, the exterior surface of the assembled nanocage, or both.

The SARS-CoV-2 binding moiety is typically an antibody or a fragment thereof and, while it can target any part of the SARS-CoV-2 virus, it typically targets the SARS-CoV-2 S glycoprotein. It will be understood that the SARS-CoV-2 binding moiety need not be an antibody or fragment thereof and may be a molecule such as a protein that binds and blocks the virus or an RBD domain in the virus, for example.

It will be understood that the antibody or fragment thereof may comprise, for example, a heavy and/or light chain of a Fab fragment. The antibody or fragment thereof may comprise a scFab fragment, a scFv fragment, a sdAb fragment, and/or a VHH region for example. It will be understood that any antibody or fragment thereof may be used in the fusion proteins described herein.

Generally, the fusion protein described herein is associated with a Fab light chain and/or heavy chain, which may be produced separately or contiguously with the fusion protein.

For example, the SARS-CoV-2 binding moiety may comprise single chain variable domain VHH-72, BD23 and/or 4A8. Alternatively or additionally, the SARS-CoV-2 binding moiety may be selected from any one or a combination of the mAbs listed in Table 4 herein. For example, the SARS-CoV-2 binding moiety may be selected from any one or a combination of mAbs 298, 324, 46, 80, 52, 82, and 236 from Table 4.

In certain aspects, the nanocage monomer described herein may be split into subunits, allowing for more SARS-CoV-2 binding moieties or other moieties to be attached thereto in various ratios. For example, in aspects, the nanocage monomer comprises a first nanocage monomer subunit linked to the SARS-CoV-2 binding moiety. In use, the first nanocage monomer subunit self-assembles with a second nanocage monomer subunit to form the nanocage monomer. As described above, a plurality of the nanocage monomers self-assemble to form a nanocage. The nanocage monomer subunits may be provided alone or in combination and may have the same or a different SARS-CoV-2 binding moiety fused thereto.

A nanocage made from the nanocage monomers and/or nanocage monomer subunits described herein may have bioactive moieties included in addition to one or more SARS-CoV-2 binding moieties.

For example, the bioactive moiety may comprise, for example, one or both chains of an Fc fragment. The Fc fragment may be derived from any type of antibody as will be understood but is, typically, an IgG1 Fc fragment. The Fc fragment may further comprise one or more mutations, such as LS, YTE, LALA, I253A, and/or LALAP, that modulate the half-life and/or effector functions of the fusion protein and/or the resulting assembled nanocage comprising the fusion protein. For example, the half-life may be in the scale of minutes, days, weeks, or even months.

Moreover, other substitutions in the fusion proteins and nanocages described herein are contemplated, including Fc sequence modifications and addition of other agents (e.g. human serum albumin peptide sequences), that allow changes in bioavailability and will be understood by a skilled person. Furthermore, the fusion proteins and nanocages described herein can be modulated in sequence or by addition of other agents to mute immunogenicity and anti-drug responses (therapeutic, e.g. matching sequence to host, or addition of immunosuppressive therapies [such as, for example, methotrexate when administering infliximab for treating rheumatoid arthritis or induction of neonatal tolerance, which is a primary strategy in reducing the incidence of inhibitors against FVIII (reviewed in: DiMichele D M, Hoots W K, Pipe S W, Rivard G E, Santagostino E. International workshop on immune tolerance induction: consensus recommendations. Haemophilia. 2007; 13:1-22, incorporated herein by reference in its entirety]).

In certain embodiments, fragment crystallizable (Fc) regions comprise an I253A mutation. In some embodiments, Fc regions further comprise the LALAP (L234A/L235A/P329G) mutations. Unless otherwise noted, numbering of mutations throughout this disclosure is according to the EU index.

In some embodiments, the Fc region is an IgG1 Fc region, (e.g., a human IgG1 Fc region), that is, except for mutations noted herein, the Fc region comprises a Fc chains that each have an amino acid sequence that is substantially similar to that of the chains within a wild type IgG1 Fc. In some embodiments, the wild type reference IgG1 Fc is a human IgG1 Fc, in which each Fc chain has an amino acid sequence of SEQ ID NO: 24.

For example, an IgG1 Fc region may comprise an Fc chain with an amino acid sequence that is at least 85%, at least 87.5%, at least 90%, at least 91%, at least 92%, at least 93%, at least 94%, at least 95%, at least 96%, at least 97%, at least 98%, or at least 99% identical to that of an Fc chain within a wild-type IgG1 Fc. In some embodiments, an IgG1 Fc region comprises an Fc chain that comprises the Fc mutations specifically described for that IgG1 Fc region, but has an amino acid sequence that is otherwise 100% identical to an Fc chain within a wild type IgG1 Fc.

In some embodiments, the Fc region is a single chain Fc (scFc), which comprises two Fc chains linked together by a covalent linker, e.g., via an amino acid linker. In some embodiments, the Fc region is an Fc monomer, which comprises a single Fc chain.

In cases where the antibody or fragment thereof comprises two chains, such as a first and second chain in the case of a Fc fragment, or a heavy and light chain, the two chains are optionally separated by a linker. The linker may be flexible or rigid, but it typically flexible to allow the chains to fold appropriately. The linker is generally long enough to impart some flexibility to the fusion protein, although it will be understood that linker length will vary depending upon the nanocage monomer and bioactive moiety sequences and the three-dimensional conformation of the fusion protein. Thus, the linker is typically from about 1 to about 130 amino acid residues, such as from about 1, 5, 10, 15, 20, 25, 30, 35, 40, 45, 50, 55, 60, 65, 70, 75, 80, 85, 90, 95, 100, 105, 110, 115, 120, or 125 to about 5, 10, 15, 20, 25, 30, 35, 40, 45, 50, 55, 60, 65, 70, 75, 80, 85, 90, 95, 100, 105, 110, 115, 120, 125, or 130 amino acid residues, such as from about 50 to about 90 amino acid residues, such as 70 amino acid residues.

The linker may be of any amino acid sequence and, in one typical example, the linker comprises a GGS repeat and, more typically, the linker comprises about 2, 3, 4, 5, or 6 GGS repeats, such as about 4 GGS repeats. In specific aspects, the linker comprises or consists of a sequence at least 70% (such as at least 75%, 80%, 85%, 90%, 95%, 96%, 97%, 98%, 99%, or 100%) identical to:
GGGGSGGGGSGGGGSGGGGSGGGGSGGGGSGGGGSGGGGSGGGGSGGGG SGGGGSGGGGSGGGGSGGGGS.

In certain embodiments, linkers are used within fusion polypeptides and/or within single-chain molecules such as scFcs. In some embodiments, the linker is an amino acid linker. For example, a linker as employed herein may comprise from about 1 to about 100 amino acid residues, e.g., about 1 to about 70, about 2 to about 70, about 1 to about 30, or about 2 to about 30 amino acid residues. In some embodiments, the linker comprises at least 2, at least 3, at least 4, at least 5, at least 6, at least 7, at least 8, at least 9, or at least 10 amino acid residues.

In certain embodiments, the linker comprises a glycine-serine sequence, e.g., a (GₙS)ₘ sequence (e.g., GGS, GGGS, or GGGGS sequence) that is present in at least 1, at least 2, at least 3, at least 4, at least 5, at least 6, at least 7, at least 8, at least 9, at least 10, at least 11, at least 12, at least 13, or at least 14 copies within the linker.

In typical aspects, the antibody or fragment thereof binds specifically to an antigen associated with SARS-CoV-2. Typically, the antigen is associated with SARS-CoV-2 and the antibody or fragment thereof comprises, for example, a binding domain from Table 4, such as binding domain 298, 52, 46, 80, 82, 236, 324 or combinations thereof.

In certain embodiments, the SARS-CoV-2-binding antibody fragment is capable of binding to the receptor binding domain (RBD) of SARS-CoV-2. In certain embodiments, the SARS-CoV-2-binding antibody fragment is capable of binding to the Spike protein (S protein) of SARS-CoV-2. In some embodiments, the SARS-CoV-2-binding antibody fragment is capable of binding to the N-terminal Domain (NTD) of the S protein of SARS-CoV-2.

In some embodiments, the SARS-CoV-2-binding antibody fragment comprises a heavy chain variable region (e.g., a V_{H} or V_{H}H). In certain embodiments, the SARS-CoV-2-binding antibody fragment comprises a heavy chain variable domain (e.g., V_{H}) and a light chain variable domain (e.g., a V_{L} or V_{K}). In certain embodiments, the SARS-CoV-2-binding antibody fragment comprises an Fab which comprises a heavy chain variable domain (e.g., V_{H}) and a light chain variable domain (e.g., a V_{L} or V_{K}).

In some embodiments, the SARS-CoV-2-binding antibody fragment comprises a V_{H} heavy chain variable domain and a V_{K} light chain variable domain. In some embodiments, the SARS-CoV-2-binding antibody fragment comprises an Fab which comprises a V_{H} heavy chain variable domain and V_{K} a light chain variable domain.

In a specific example, the antibody or fragment thereof comprises or consists of a sequence at least 70% (such as at least 75%, 80%, 85%, 90%, 95%, 96%, 97%, 98%, 99%, or 100%) identical to one or more of the following sequences:

Fc chain 1:
DKTHTCPPCPAPELLGGPSVFLFPPKPKDTLMISRTPEVTCVVVD
VSHEDPEVKFNWYVDGVEVHNAKTKPREEQYNSTYRWSVLTVLHQ
DWLNGKEYKCKVSNKALPAPIEKTISKAKGQPREPQVYTLPPSRE
EMTKNQVSLTCLVKGFYPSDIAVEWESNGQPENNYKTTPPVLDSD
GSFFLYSKLTVDKSRWQQGNVFSCSVLHEALHSHYTQKSLSLSPGK;
Fc chain 2:
DKTHTCPPCPAPELLGGPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
EEMTKNQVSLTCLVKGFYPSDIAVEWESNGQPENNYKTTPPVLDS
DGSFFLYSKLTVDKSRWQQGNVFSCSVLHEALHSHYTQKSLSLSP
GK;
298 light chain
DIVMTQSPDSLAVSLGERATINCKSSQSVLYSSNNKNYLAWYQQK
PGQPPKLLIYWASTRESGVPDRFSGSGSGTDFTLTISSLQAEDVA
VYYCQQYYSTPPTFGQGTKLEIKRTVAAPSVFIFPPSDEQLKSGT
ASVVCLLNNFYPREAKVQWKVDNALQSGNSQESVTEQDSKDSTYS
LSSTLTLSKADYEKHKVYACEVTHQGLSSPVTKSFNRGEC
298 Fab heavy chain
QVQLVQSGAEVKKPGASVKVSCKASGGTFSTYGISWVRQAPGQGL
EWMGWISPNSGGTDLAQKFQGRVTMTRDTSTSTVYMELSSLRSED
TAVYYCASDPRDDIAGGYWGQGTLVTVSSASTKGPSVFPLAPSSK
STSGGTAALGCLVKDYFPEPVTVSWNSGALTSGVHTFPAVLQSSG
LYSLSSVTVPSSSLGTQTYICNVNHKPSNTKVDKKVEPKSC
52 light chain
DIQMTQSPSSLSASVGDRVTITCRASQGISNNLNWYQQKPGKAPK
LLIYAASSLESGVPSRFSGSGSGTDFTLTISSLQPEDFATYYCQQ
GNGFPLTFGPGTKVDIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
52 Fab heavy chain
QVQLVQSGAEVKKPGSSVKVSCKASGYTFTSYGISWVRQAPGQGL
EWMGGIIPMFGTTNYAQKFQGRVTITADKSTSTAYMELSSLRSED
TAVYYCARDRGDTIDYWGQGTLVTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKKVEPKSC
46 light chain
DIQMTQSPSSLSASVGDRVTITCRASQSISSWLAWYQQKPGKAPK
LLIYDASNLETGVPSRFSGSGSGTDFTLTISSLQPEDFATYYCQQ
SYSTPFTFGPGTKVDIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
46 Fab heavy chain
EVQLLESGGGLVQPGRSLRLSCAASGFTFSSYAMSWVRQAPGKGL
EWVSTIYSGGSTYYADSVKGRFTISRDNSKNTLYLQMNSLRAEDT
AVYYCARGDSRDAFDIWGQGTMVTVSSASTKGPSVFPLAPSSKST
SGGTAALGCLVKDYFPEPVTVSWNSGALTSGVHTFPAVLQSSGLY
SLSSWTVPSSSLGTQTYICNVNHKPSNTKVDKKVEPKSC
80 light chain
DIVMTQSPDSLAVSLGERATINCKSSQSVLYSSNNKNYLAWYQQK
PGQPPKLLIYWASTRESGVPDRFSGSGSGTDFTLTISSLQAEDVA
VYYCQQYYSAPLTFGGGTKVEIKRTVAAPSVFIFPPSDEQLKSGT
ASWVCLLNNFYPREAKVQWKVDNALQSGNSQESVTEQDSKDSTYS
LSSTLTLSKADYEKHKVYACEVTHQGLSSPVTKSFNRGEC
80 Fab heavy chain
QVQLVQSGAEVKKPGSSVKVSCKASGGTFNRYAFSWVRQAPGQGL
EWMGGIIPIFGTANYAQKFQGRVTITADESTSTAYMELSSLRSED
TAVYYCARSTRELPEVVDWYFDLWGRGTLVTVSSASTKGPSVFPL
APSSKSTSGGTAALGCLVKDYFPEPVTVSWNSGALTSGVHTFPAV
LQSSGLYSLSSVVTVPSSSLGTQTYICNVNHKPSNTKVDKKVEPK
SC
82 light chain
DIQMTQSPSSLSASVGDRVTITCRASQVISNYLAWYQQKPGKAPK
LLIYDASNLETGVPSRFSGSGSGTDFTLTISSLQPEDFATYYCQQ
SFSPPPTFGQGTRLEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
82 Fab heavy chain
QVQLVQSGAEVKKPGASVKVSCKASGGSFSTSAFYWVRQAPGQGL
EWMGWINPYTGGTNYAQKFQGRVTMTRDTSTSTVYMELSSLRSED
TAVYYCARSRALYGSGSYFDYWGQGTLVTVSSASTKGPSVFPLAP
SSKSTSGGTAALGCLVKDYFPEPVTVSWNSGALTSGVHTFPAVLQ
SSGLYSLSSVVTVPSSSLGTQTYICNVNHKPSNTKVDKKVEPKSC
236 light chain
DIVMTQSPLSLPVTPGEPASISCRSSQSLLHSNGYNYLDWYLQKP
GQSPQLLIYLGSNRASGVPDRFSGSGSGTDFTLKISRVEAEDVGV
YYCMQALQTPPTFGQGTRLEIKRTVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSFNRGEC
236 Fab heavy chain
QVQLVQSGAEVKKPGASVKVSCKASGGTFTSYGINWVRQAPGQGL
EWMGWMNPNSGNTGYAQKFQGRVTMTRDTSTSTVYMELSSLRSED
TAVYYCASRGIQLLPRGMDVWGQGTTVTVSSASTKGPSVFPLAPS
SKSTSGGTAALGCLVKDYFPEPVTVSWNSGALTSGVHTFPAVLQS
SGLYSLSSVVTVPSSSLGTQTYICNVNHKPSNTKVDKKVEPKSC
324 light chain
DIQMTQSPSSLSASVGDRVTITCRASQSITTYLNWYQQKPGKAPK
LLIYDASNLETGVPSRFSGSGSGTDFTLTISSLQPEDFATYYCQQ
SYSTPPTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
324 Fab heavy chain
QVQLVQSGAEVKKPGASVKVSCKASGGTFNNYGISWVRQAPGQGL
EWMGWMNPNSGNTGYAQKFQGRVTMTRDTSTSTVYMELSSLRSED
TAVYYCARVGDYGDYIVSPFDLWGRGTLVTVSSASTKGPSVFPLA
PSSKSTSGGTAALGCLVKDYFPEPVTVSWNSGALTSGVHTFPAVL
QSSGLYSLSSVVTVPSSSLGTQTYICNVNHKPSNTKVDKKVEPKS
C

or combinations thereof.

In further aspects, the antibody or fragment thereof is conjugated to or associated with a further moiety, such as a detectable moiety (e.g., a small molecule, fluorescent molecule, radioisotope, or magnetic particle), a pharmaceutical agent, a diagnostic agent, or combinations thereof and may comprise, for example, an antibody-drug conjugate.

In aspects wherein the bioactive moiety is a detectable moiety, the detectable moiety may comprise a fluorescent protein, such as GFP, EGFP, Ametrine, and/or a flavin-based fluorescent protein, such as a LOV-protein, such as iLOV.

In aspects wherein the bioactive moiety is a pharmaceutical agent, the pharmaceutical agent may comprise for example, a small molecule, peptide, lipid, carbohydrate, or toxin.

In typical aspects, the nanocage assembled from the fusion proteins described herein comprises from about 3 to about 100 nanocage monomers, such as from about 3, 4, 5, 6, 7, 8, 9, 10, 12, 14, 16, 18, 20, 22, 24, 26, 28, 30, 32, 34, 36, 38, 40, 42, 44, 46, 48, 50, 52, 55, 56, 58, 60, 62, 64, 66, 68, 70, 72, 74, 76, 78, 80, 82, 84, 86, 88, 90, 92, 94, 96, or 98 to about 4, 5, 6, 7, 8, 9, 10, 12, 14, 16, 18, 20, 22, 24, 26, 28, 30, 32, 34, 36, 38, 40, 42, 44, 46, 48, 50, 52, 55, 56, 58, 60, 62, 64, 66, 68, 70, 72, 74, 76, 78, 80, 82, 84, 86, 88, 90, 92, 94, 96, 98, or 100 nanocage monomers, such as 24, 32, or 60 monomers. The nanocage monomer may be any known nanocage monomer, natural, synthetic, or partly synthetic and is, in aspects, selected from ferritin, apoferritin, encapsulin, SOR, lumazine synthase, pyruvate dehydrogenase, carboxysome, vault proteins, GroEL, heat shock protein, E2P, MS2 coat protein, fragments thereof, and variants thereof. Typically, the nanocage monomer is ferritin or apoferritin.

When apoferritin is chosen as the nanocage monomer, typically the first and second nanocage monomer subunits interchangeably comprise the “N” and “C” regions of apoferritin. It will be understood that other nanocage monomers can be divided into bipartite subunits much like apoferritin as described herein so that the subunits self-assemble and are each amenable to fusion with a bioactive moiety.

In some embodiments, the nanocage monomer is a ferritin monomer. The term “ferritin monomer,” is used herein to refer to a single chain of a ferritin that, in the presence of other ferritin chains, is capable of self-assembling into a polypeptide complex comprising a plurality of ferritin chains. In some embodiments, ferritin chains self-assembled into a polypeptide complex comprising 24 or more ferritin chains. In some embodiments, the ferritin monomer is a ferritin light chain. In some embodiments, the ferritin monomer does not include a ferritin heavy chain or other ferritin components capable of binding to iron.

In some embodiments, each fusion polypeptide within the self-assembled polypeptide complex comprises a ferritin light chain or a subunit of a ferritin light chain. In these embodiments, the self-assembled polypeptide complex does not comprise any ferritin heavy chains or subunits of ferritin heavy chains.

In some embodiments, the ferritin monomer is a human ferritin chain, e.g., a human ferritin light chain, e.g., a human ferritin light chain having the sequence of at least residues 2-175 of SEQ ID NO:1. In some embodiments, the ferritin monomer is a mouse ferritin chain.

A “subunit” of a ferritin monomer refers to a portion of a ferritin monomer that is capable of spontaneously associating with another, distinct subunit of a ferritin monomer, so that the subunits together form a ferritin monomer, which ferritin monomer, in turn, is capable of self-assembling with other ferritin monomers to form a polypeptide complex.

In some embodiments, the ferritin monomer subunit comprises approximately half of a ferritin monomer. As used herein, the term “N-half ferritin” refers to approximately half of a ferritin chain, which half comprises the N-terminus of the ferritin chain. As used herein, the term “C-half ferritin” refers to approximately half a ferritin chain, which half comprises the C-terminus of the ferritin chain. The exact point at which a ferritin chain may be divided to form the N-half ferritin and the C-half ferritin may vary depending on the embodiment. In the context of ferritin monomer subunits based on human ferritin light chain, for example, the halves may divided at a point that corresponds to a position between about position 75 to about position 100 of SEQ ID NO:1. For example, in some embodiments, an N-half ferritin based on a human ferritin light chain has an amino acid sequence corresponding to residues 1-95 of SEQ ID NO:1 (or a substantial portion thereof), and a C-half ferritin based on a human ferritin light chain has an amino acid sequence corresponding to residues 96-175 of SEQ ID NO:1 (or a substantial portion thereof).

In some embodiments, the halves are divided at a point that corresponds to a position between about position 85 to about position 92 of SEQ ID NO:1. For example, in some embodiments, an N-half ferritin based on a human ferritin light chain has an amino acid sequence corresponding to residues 1-90 of SEQ ID NO:1 (or a substantial portion thereof), and a C-half ferritin based on a human ferritin light chain has an amino acid sequence corresponding to residues 91-175 of SEQ ID NO:1 (or a substantial portion thereof).

Typically, the “N” region of apoferritin comprises or consists of a sequence at least 70% (such as at least 75%, 80%, 85%, 90%, 95%, 96%, 97%, 98%, 99%, or 100%) identical to:
MSSQIRQNYSTDVEAAVNSLVNLYLQASYTYLSLGFYFDRDDVALEGVSHFFRELA EEKREGYERLLKMQNQRGGRALFQDIKKPAEDEW.

Typically, the “C” region of apoferritin comprises or consists of a sequence at least 70% (such as at least 75%, 80%, 85%, 90%, 95%, 96%, 97%, 98%, 99%, or 100%) identical to:
GKTPDAMKAAMALEKKLNQALLDLHALGSARTDPHLCDFLETHFLDEEVKLIKKMG DHLTNLHRLGGPEAGLGEYLFERLTLRHD

or
GKTPDAMKAAMALEKKLNQALLDLHALGSARTDPHLCDFLETHFLDEEVKLIKKMG DHLTNLHRLGGPEAGLGEYLFERLTLKHD.

In aspects, the fusion protein described herein, further comprises a linker between the nanocage monomer subunit and the bioactive moiety, much like the linker described above. Again, the linker may be flexible or rigid, but it typically flexible to allow the bioactive moiety to retain activity and to allow the pairs of nanocage monomer subunits to retain self-assembly properties. The linker is generally long enough to impart some flexibility to the fusion protein, although it will be understood that linker length will vary depending upon the nanocage monomer and bioactive moiety sequences and the three-dimensional conformation of the fusion protein. Thus, the linker is typically from about 1 to about 30 amino acid residues, such as from about 1, 2, 3, 4, 5, 6, 7, 8, 9, 10, 11, 12, 13, 14, 15, 16, 17, 18, 19, 20, 21, 22, 23, 24, 25, 26, 27, 28, or 29 to about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues, such as from about 8 to about 16 amino acid residues, such as 8, 10, or 12 amino acid residues.

The linker may be of any amino acid sequence and, in one typical example, the linker comprises a GGS repeat and, more typically, the linker comprises about 2, 3, 4, 5, or 6 GGS repeats, such as about 4 GGS repeats. In specific aspects, the linker comprises or consists of a sequence at least 70% (such as at least 75%, 80%, 85%, 90%, 95%, 96%, 97%, 98%, 99%, or 100%) identical to:
GGGGSGGGGSGGGGSGGGGSGGGGSGG

Similarly, the fusion protein may further comprising a C-terminal linker for improving one or more attributes of the fusion protein. In aspects, the comprises a GGS repeat and, more typically, the linker comprises about 2, 3, 4, 5, or 6 GGS repeats, such as about 4 GGS repeats. In specific aspects, the C-terminal linker comprises or consists of a sequence at least 70% (such as at least 75%, 80%, 85%, 90%, 95%, 96%, 97%, 98%, 99%, or 100%) identical to:
GGSGGSGGSGGSGGGSGGSGGSGGSG

Also described herein is a pair of the fusion proteins described above, wherein the pair self-assembles to form a nanocage monomer, wherein the first and second nanocage monomer subunits are fused to different SARS-CoV-2 binding moieties. This provides multivalency and/or multispecificity to a single nanocage monomer assembled from the pair of subunits.

A substantially identical sequence may comprise one or more conservative amino acid mutations. It is known in the art that one or more conservative amino acid mutations to a reference sequence may yield a mutant peptide with no substantial change in physiological, chemical, or functional properties compared to the reference sequence; in such a case, the reference and mutant sequences would be considered “substantially identical” polypeptides. Conservative amino acid mutation may include addition, deletion, or substitution of an amino acid; a conservative amino acid substitution is defined herein as the substitution of an amino acid residue for another amino acid residue with similar chemical properties (e.g. size, charge, or polarity).

In a non-limiting example, a conservative mutation may be an amino acid substitution. Such a conservative amino acid substitution may substitute a basic, neutral, hydrophobic, or acidic amino acid for another of the same group. By the term “basic amino acid” it is meant hydrophilic amino acids having a side chain pK value of greater than 7, which are typically positively charged at physiological pH. Basic amino acids include histidine (His or H), arginine (Arg or R), and lysine (Lys or K). By the term “neutral amino acid” (also “polar amino acid”), it is meant hydrophilic amino acids having a side chain that is uncharged at physiological pH, but which has at least one bond in which the pair of electrons shared in common by two atoms is held more closely by one of the atoms. Polar amino acids include serine (Ser or S), threonine (Thr or T), cysteine (Cys or C), tyrosine (Tyr or Y), asparagine (Asn or N), and glutamine (Gln or Q). The term “hydrophobic amino acid” (also “non-polar amino acid”) is meant to include amino acids exhibiting a hydrophobicity of greater than zero according to the normalized consensus hydrophobicity scale of Eisenberg (1984). Hydrophobic amino acids include proline (Pro or P), isoleucine (Ile or I), phenylalanine (Phe or F), valine (Val or V), leucine (Leu or L), tryptophan (Trp or W), methionine (Met or M), alanine (Ala or A), and glycine (Gly or G).

“Acidic amino acid” refers to hydrophilic amino acids having a side chain pK value of less than 7, which are typically negatively charged at physiological pH. Acidic amino acids include glutamate (Glu or E), and aspartate (Asp or D).

Sequence identity is used to evaluate the similarity of two sequences; it is determined by calculating the percent of residues that are the same when the two sequences are aligned for maximum correspondence between residue positions. Any known method may be used to calculate sequence identity; for example, computer software is available to calculate sequence identity. Without wishing to be limiting, sequence identity can be calculated by software such as NCBI BLAST2 service maintained by the Swiss Institute of Bioinformatics (and as found at ca.expasy.org/tools/blast/), BLAST-P, Blast-N, or FASTA-N, or any other appropriate software that is known in the art.

The substantially identical sequences of the present invention may be at least 85% identical; in another example, the substantially identical sequences may be at least 70, 75, 80, 85, 90, 95, 96, 97, 98, 99, or 100% (or any percentage there between) identical at the amino acid level to sequences described herein. In specific aspects, the substantially identical sequences retain the activity and specificity of the reference sequence. In a non-limiting embodiment, the difference in sequence identity may be due to conservative amino acid mutation(s).

The polypeptides or fusion proteins of the present invention may also comprise additional sequences to aid in their expression, detection or purification. Any such sequences or tags known to those of skill in the art may be used. For example, and without wishing to be limiting, the fusion proteins may comprise a targeting or signal sequence (for example, but not limited to ompA), a detection tag, exemplary tag cassettes include Strep tag, or any variant thereof; see, e.g., U.S. Pat. No. 7,981,632, His tag, Flag tag having the sequence motif DYKDDDDK, Xpress tag, Avi tag, Calmodulin tag, Polyglutamate tag, HA tag, Myc tag, Nus tag, S tag, SBP tag, Softag 1, Softag 3, V5 tag, CREB-binding protein (CBP), glutathione S-transferase (GST), maltose binding protein (MBP), green fluorescent protein (GFP), Thioredoxin tag, or any combination thereof; a purification tag (for example, but not limited to a His₅ or His₆), or a combination thereof.

In another example, the additional sequence may be a biotin recognition site such as that described by Cronan et al in WO 95/04069 or Voges et al in WO/2004/076670. As is also known to those of skill in the art, linker sequences may be used in conjunction with the additional sequences or tags.

More specifically, a tag cassette may comprise an extracellular component that can specifically bind to an antibody with high affinity or avidity. Within a single chain fusion protein structure, a tag cassette may be located (a) immediately amino-terminal to a connector region, (b) interposed between and connecting linker modules, (c) immediately carboxy-terminal to a binding domain, (d) interposed between and connecting a binding domain (e.g., scFv or scFab) to an effector domain, (e) interposed between and connecting subunits of a binding domain, or (f) at the amino-terminus of a single chain fusion protein. In certain embodiments, one or more junction amino acids may be disposed between and connecting a tag cassette with a hydrophobic portion, or disposed between and connecting a tag cassette with a connector region, or disposed between and connecting a tag cassette with a linker module, or disposed between and connecting a tag cassette with a binding domain.

Also encompassed herein are isolated or purified fusion proteins, polypeptides, or fragments thereof immobilized onto a surface using various methodologies; for example, and without wishing to be limiting, the polypeptides may be linked or coupled to the surface via His-tag coupling, biotin binding, covalent binding, adsorption, and the like. The solid surface may be any suitable surface, for example, but not limited to the well surface of a microtiter plate, channels of surface plasmon resonance (SPR) sensorchips, membranes, beads (such as magnetic-based or sepharose-based beads or other chromatography resin), glass, a film, or any other useful surface.

In other aspects, the fusion proteins may be linked to a cargo molecule; the fusion proteins may deliver the cargo molecule to a desired site and may be linked to the cargo molecule using any method known in the art (recombinant technology, chemical conjugation, chelation, etc.). The cargo molecule may be any type of molecule, such as a therapeutic or diagnostic agent.

In some aspects, the cargo molecule is a protein and is fused to the fusion protein such that the cargo molecule is contained in the nanocage internally. In other aspects, the cargo molecule is not fused to the fusion protein and is contained in the nanocage internally. The cargo molecule is typically a protein, a small molecule, a radioisotope, or a magnetic particle.

The fusion proteins described herein specifically bind to their targets. Antibody specificity, which refers to selective recognition of an antibody for a particular epitope of an antigen, of the antibodies or fragments described herein can be determined based on affinity and/or avidity. Affinity, represented by the equilibrium constant for the dissociation of an antigen with an antibody (K_{D}), measures the binding strength between an antigenic determinant (epitope) and an antibody binding site. Avidity is the measure of the strength of binding between an antibody with its antigen. Antibodies typically bind with a K_{D} of 10⁻⁵ to 10⁻¹¹ M. Any K_{D} greater than 10⁻⁴ M is generally considered to indicate non-specific binding. The lesser the value of the K_{D}, the stronger the binding strength between an antigenic determinant and the antibody binding site. In aspects, the antibodies described herein have a K_{D} of less than 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, 10⁻¹⁴ M, or 10⁻¹⁵ M.

Also described herein are nanocages comprising at least one fusion protein described herein and at least one second nanocage monomer subunit that self-assembles with the fusion protein to form a nanocage monomer. Further, pairs of the fusion proteins are described herein, wherein the pair self-assembles to form a nanocage monomer and wherein the first and second nanocage monomer subunits are fused to different bioactive moieties.

It will be understood that the nanocages may self-assemble from multiple identical fusion proteins, from multiple different fusion proteins (and therefore be multivalent and/or multispecific), from a combination of fusion proteins and wild-type proteins, and any combination thereof. For example, the nanocages may be decorated internally and/or externally with at least one of the fusion proteins described herein in combination with at least one anti-SARS-CoV-2 antibody. In typical aspects, from about 20% to about 80% of the nanocage monomers comprise the fusion protein described herein. In view of the modular solution described herein, the nanocages could in theory comprise up to twice as many bioactive moieties as there are monomers in the nanocage, as each nanocage monomer may be divided into two subunits, each of which can independently bind to a different bioactive moiety. It will be understood that this modularity can be harnessed to achieve any desired ratio of bioactive moieties as described herein in specific example to a 4:2:1:1 ratio of four different bioactive moieties. For example, the nanocages described herein may comprise at least 2, 3, 4, 5, 6, 7, 8, 9, or 10 different bioactive moieties. In this way, the nanocages can be multivalent and/or multispecific and the extent of this can be controlled with relative ease.

In aspects, the nanocages described herein may further comprise at least one whole nanocage monomer, optionally fused to a bioactive moiety that may be the same or different from the bioactive moiety described herein as being linked to a nanocage monomer subunit.

In typical aspects, the nanocages described herein comprise a first, second, and third fusion protein to a subunit or the monomer, and optionally at least one whole nanocage monomer, optionally fused to a bioactive moiety, wherein the bioactive moieties of the first, second, and third fusion proteins and of the whole nanocage monomer are all different from one another.

More typically, the first, second, and third fusion proteins each comprise an antibody or Fc fragment thereof fused to N- or C-half ferritin, wherein at least one of the first, second, and third fusion proteins is fused to N-half ferritin and at least one of the first, second, and third fusion proteins is fused to C-half ferritin. For example, the antibody or fragment thereof of the first fusion protein is typically an Fc fragment; the second and third fusion proteins typically each comprise an antibody or fragment thereof specific for a different antigen of a virus such as SARS-CoV-2 and the whole nanocage monomer is fused to a bioactive moiety that is specific for another different antigen, optionally of the same virus such as SARS-CoV-2.

In aspects, the antibody or fragment thereof of the second fusion protein is 46 or 52; and the antibody or fragment thereof of the third fusion protein is 324 or 80. In a typical aspect, the nanocage described herein comprises the following four fusion proteins, optionally in a 4:2:1:1: ratio:
a. 298 (optionally sc298) fused to full length ferritin;
b. Fc (optionally scFc) fused to N-ferritin;
c. 46 or 52 (optionally sc46 or sc52) fused to C-ferritin; and
d. 324 or 80 (optionally sc324 or sc80) fused to C-ferritin.

In aspects, the nanocage described herein comprises or consists of sequences at least 70% (such as at least 75%, 80%, 85%, 90%, 95%, 96%, 97%, 98%, 99%, or 100%) identical to one or more of the following sequences, where ferritin subunits are in bold, linkers are underlined, light chains are italicized, and heavy chains are in lowercase:

a. 298-hFerr:
DIVMTQSPDSLAVSLGERATINCKSSQSVLYSSNNKNYLAWYQQK
PGQPPKLLIYWASTRESGVPDRFSGSGSGTDFTLTISSLQAEDVA
VYYCQQYYSTPPTFGQGTKLEIKRTVAAPSVFIFPPSDEQLKSGT
ASVVCLLNNFYPREAKVQWKVDNALQSGNSQESVTEQDSKDSTYS
LSSTLTLSKADYEKHKVYACEVTHQGLSSPVTKSFNRGECGGGGS
GGGGSGGGGSGGGGSGGGGSGGGGSGGGGSGGGGSGGGGSGGGGS
GGGGSGGGGSGGGGSGGGGSQVQLVQSGAEVKKPGASVKVSCKAS
GGTFSTYGISWVRQAPGQGLEWMGWISPNSGGTDLAQKFQGRVTM
TRDTSTSTVYMELSSLRSEDTAVYYCASDPRDDIAGGYWGQGTLV
TVSSASTKGPSVFPLAPSSKSTSGGTAALGCLVKDYFPEPVTVSW
NSGALTSGVHTFPAVLQSSGLYSLSSVVTVPSSSLGTQTYICNVN
HKPSNTKVDKKVEPKSCDGGGGSGGGGSGGGGSGGGGSGGGGSGG
MSSQIRQNYSTDVEAAVNSLVNLYLQASYTYLSLGFYFDRDDVAL
EGVSHFFRELAEEKREGYERLLKMQNQRGGRALFQDIKKPAEDEW
GKTPDAMKAAMALEKKLNQALLDLHALGSARTDPHLCDFLETHFL
DEEVKLIKKMGDHLTNLHRLGGPEAGLGEYLFERLTLRHD
or
DIVMTQSPDSLAVSLGERATINCKSSQSVLYSSNNKNYLAWYQQK
PGQPPKLLIYWASTRESGVPDRFSGSGSGTDFTLTISSLQAEDVA
VYYCQQYYSTPPTFGQGTKLEIKRTVAAPSVFIFPPSDEQLKSGT
ASVVCLLNNFYPREAKVQWKVDNALQSGNSQESVTEQDSKDSTYS
LSSTLTLSKADYEKHKVYACEVTHQGLSSPVTKSFNRGECGGGGS
GGGGSGGGGSGGGGSGGGGSGGGGSGGGGSGGGGSGGGGSGGGGS
GGGGSGGGGSGGGGSGGGGSQVQLVQSGAEVKKPGASVKVSCKAS
GGTFSTYGISWVRQAPGQGLEWMGWISPNSGGTDLAQKFQGRVTM
TRDTSTSTVYMELSSLRSEDTAVYYCASDPRDDIAGGYWGQGTLV
TVSSASTKGPSVFPLAPSSKSTSGGTAALGCLVKDYFPEPVTVSW
NSGALTSGVHTFPAVLQSSGLYSLSSVVTVPSSSLGTQTYICNVN
HKPSNTKVDKKVEPKSCDGGGGSGGGGSGGGGSGGGGSGGGGSGG
SSQIRQNYSTDVEAAVNSLVNLYLQASYTYLSLGFYFDRDDVALE
GVSHFFRELAEEKREGYERLLKMQNQRGGRALFQDIKKPAEDEWG
KTPDAMKAAMALEKKLNQALLDLHALGSARTDPHLCDFLETHFLD
EEVKLIKKMGDHLTNLHRLGGPEAGLGEYLFERLTLRHD
b. Fc-N-hFerr LALAP 1253A
DKTHTCPPCPAPEAAGGPSVFLFPPKPKDTLMASRTPEVTCVVVD
VSHEDPEVKFNWYVDGVEVHNAKTKPREEQYNSTYRVVSVLTVLH
QDWLNGKEYKCKVSNKALGAPIEKTISKAKGQPREPQVYTLPPSR
EEMTKNQVSLTCLVKGFYPSDIAVEWESNGQPENNYKTTPPVLDS
DGSFFLYSKLTVDKSRWQQGNVFSCSVMHEALHNHYTQKSLSLSP
GKGGGGSGGGGSGGGGSGGGGSGGGGGGGGSGGGGSGGGGSGGGG
SGGGGSGGGGSGGGGSGGGGSGGGGSDKTHTCPPCPAPEAAGGPS
VFLFPPKPKDTLMASRTPEVTCVVVDVSHEDPEVKFNWYVDGVEV
HNAKTKPREEQYNSTYRVVSVLTVLHQDWLNGKEYKCKVSNKALG
APIEKTISKAKGQPREPQVYTLPPSREEMTKNQVSLTCLVKGFYP
SDIAVEWESNGQPENNYKTTPPVLDSDGSFFLYSKLTVDKSRWQQ
GNVFSCSVMHEALHNHYTQKSLSLSPGKGGGGSGGGGSGGGGSGG
GGSGGGGSGGMSSQIRQNYSTDVEAAVNSLVNLYLQASYTYLSLG
FYFDRDDVALEGVSHFFRELAEEKREGYERLLKMQNQRGGRALFQ
DIKKPAEDEW
c1. 52-C-hFerr
DIQMTQSPSSLSASVGDRVTITCRASQGISNNLNWYQQKPGKAPK
LLIYAASSLESGVPSRFSGSGSGTDFTLTISSLQPEDFATYYCQQ
GNGFPLTFGPGTKVDIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGECGGGGSGGGGSG
GGGSGGGGSGGGGSGGGGSGGGGSGGGGSGGGGSGGGGSGGGGSG
GGGSGGGGSGGGGSQVQLVQSGAEVKKPGSSVKVSCKASGYTFTS
YGISWVRQAPGQGLEWMGGIIPMFGTTNYAQKFQGRVTITADKST
STAYMELSSLRSEDTAVYYCARDRGDTIDYWGQGTLVTVSSASTK
GPSVFPLAPSSKSTSGGTAALGCLVKDYFPEPVTVSWNSGALTSG
VHTFPAVLQSSGLYSLSSVVTVPSSSLGTQTYICNVNHKPSNTKV
DKKVEPKSCDGGGGSGGGGSGGGGSGGGGSGGGGSGGGKTPDAMK
AAMALEKKLNQALLDLHALGSARTDPHLCDFLETHFLDEEVKLIK
KMGDHLTNLHRLGGPEAGLGEYLFERLTLRHD
c2. 46-C-hFerr
DIQMTQSPSSLSASVGDRVTITCRASQSISSWLAWYQQKPGKAPK
LLIYDASNLETGVPSRFSGSGSGTDFTLTISSLQPEDFATYYCQQ
SYSTPFTFGPGTKVDIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGECGGGGGGGGSGG
GGGGGGSGGGGSGGGGGGGGSGGGGGGGGSGGGGSGGGGSGGGGS
GGGGSGGGGSEVQLLESGGGLVQPGRSLRLSCAASGFTFSSYAMS
WVRQAPGKGLEWVSTIYSGGSTYYADSVKGRFTISRDNSKNTLYL
QMNSLRAEDTAVYYCARGDSRDAFDIWGQGTMVTVSSASTKGPSV
FPLAPSSKSTSGGTAALGCLVKDYFPEPVTVSWNSGALTSGVHTF
PAVLQSSGLYSLSSVVTVPSSSLGTQTYICNVNHKPSNTKVDKKV
EPKSCDGGGGSGGGGSGGGGSGGGGSGGGGSGGGKTPDAMKAAMA
LEKKLNQALLDLHALGSARTDPHLCDFLETHFLDEEVKLIKKMGD
HLTNLHRLGGPEAGLGEYLFERLTLRHD
d1. 324-C-hFerr
DIQMTQSPSSLSASVGDRVTITCRASQSITTYLNWYQQKPGKAPK
LLIYDASNLETGVPSRFSGSGSGTDFTLTISSLQPEDFATYYCQQ
SYSTPPTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGECGGGGSGGGGSG
GGGSGGGGSGGGGSGGGGSGGGGSGGGGSGGGGSGGGGSGGGGSG
GGGSGGGGSGGGGSQVQLVQSGAEVKKPGASVKVSCKASGGTFNN
YGISWVRQAPGQGLEWMGWMNPNSGNTGYAQKFQGRVTMTRDTST
STVYMELSSLRSEDTAVYYCARVGDYGDYIVSPFDLWGRGTLVTV
SSASTKGPSVFPLAPSSKSTSGGTAALGCLVKDYFPEPVTVSWNS
GALTSGVHTFPAVLQSSGLYSLSSVVTVPSSSLGTQTYICNVNHK
PSNTKVDKKVEPKSCDGGGGSGGGGSGGGGSGGGGSGGGGSGGGK
TPDAMKAAMALEKKLNQALLDLHALGSARTDPHLCDFLETHFLDE
EVKLIKKMGDHLTNLHRLGGPEAGLGEYLFERLTLRHD
d2. 80-C-hFerr
DIVMTQSPDSLAVSLGERATINCKSSQSVLYSSNNKNYLAWYQQK
PGQPPKLLIYWASTRESGVPDRFSGSGSGTDFTLTISSLQAEDVA
VYYCQQYYSAPLTFGGGTKVEIKRTVAAPSVFIFPPSDEQLKSGT
ASVVCLLNNFYPREAKVQWKVDNALQSGNSQESVTEQDSKDSTYS
LSSTLTLSKADYEKHKVYACEVTHQGLSSPVTKSFNRGECGGGGS
GGGGSGGGGSGGGGSGGGGSGGGGSGGGGSGGGGSGGGGSGGGGS
GGGGSGGGGSGGGGSGGGGSQVQLVQSGAEVKKPGSSVKVSCKAS
GGTFNRYAFSWVRQAPGQGLEWMGGIIPIFGTANYAQKFQGRVTI
TADESTSTAYMELSSLRSEDTAVYYCARSTRELPEVVDWYFDLWG
RGTLVTVSSASTKGPSVFPLAPSSKSTSGGTAALGCLVKDYFPEP
VTVSWNSGALTSGVHTFPAVLQSSGLYSLSSVVTVPSSSLGTQTY
ICNVNHKPSNTKVDKKVEPKSCDGGGGGGGGSGGGGSGGGGSGGG
GSGGGKTPDAMKAAMALEKKLNQALLDLHALGSARTDPHLCDFL
ETHFLDEEVKLIKKMGDHLTNLHRLGGPEAGLGEYLFERLTLRHD

In one aspect, provided are self-assembled polypeptide complexes comprising a plurality of fusion polypeptides as disclosed herein. In many embodiments, self-assembled polypeptide complexes comprise (1) a plurality of first fusion polypeptides, each first fusion polypeptide comprising an Fc region linked to a nanocage monomer (e.g., ferritin monomer, e.g., human ferritin monomer, or subunit thereof), as disclosed herein; and (2) a plurality of second fusion polypeptides, each second fusion polypeptide comprising a SARS-CoV-2-binding antibody fragment (e.g., an Fab fragment of an antibody that is capable of binding to SARS-CoV-2 protein (e.g., the Spike protein or a receptor-binding domain (RBD))), the SARS-CoV-2-binding antibody fragment being linked to a nanocage monomer (e.g., ferritin monomer, e.g., human ferritin monomer) or subunit thereof. In some embodiments, self-assembled polypeptide complex further comprises a plurality of third fusion polypeptides, each third fusion polypeptide being distinct from the second fusion polypeptide and each comprising (1) a nanocage monomer (e.g., ferritin monomer, e.g., human ferritin monomer) linked to (2) a SARS-CoV-2-binding antibody fragment (e.g., Fab fragment of an antibody that is capable of binding to a SARS-CoV-2 protein).

In some embodiments, one of the fusion polypeptides (e.g., the first fusion polypeptide or the second fusion polypeptide) comprises an N-half nanocage monomer (e.g., an N-half ferritin) (but not a full-length nanocage (e.g., ferritin) monomer), and one of the other fusion polypeptides comprises a C-half nanocage monomer (e.g., a C-half ferritin) (but not a full-length nanocage (e.g., ferritin) monomer). In many of these embodiments, the ratio of fusion polypeptides comprising the N-half nanocage monomer (e.g., N-half ferritin) to the fusion polypeptides comprising the C-half nanocage monomer (e.g., C-half ferritin) within the self-assembled polypeptide complex is about 1:1.

In some embodiments, the self-assembled polypeptide complex comprises 24 fusion polypeptides. In some embodiments, the self-assembled polypeptide complex comprises more than 24 fusion polypeptides, e.g., at least 26, at least 28, at least 30, at least 32 fusion polypeptides, at least 34 fusion polypeptides, at least 36 fusion polypeptides, at least 38 fusion polypeptides, at least 40 fusion polypeptides, at least 42 fusion polypeptides, at least 44 fusion polypeptides, at least 46 fusion polypeptides, or at least 48 fusion polypeptides. In some embodiments, the self-assembled polypeptide complex comprises 32 fusion polypeptides.

In some embodiments, the self-assembled polypeptide complex comprises at least 4, at least 5, least 6, at least 7, or at least 8 first fusion polypeptides.

In some embodiments, the self-assembled polypeptide complex comprises at least 4, at least 5, least 6, at least 7, or at least 8 second fusion polypeptides.

In some embodiments, the self-assembled polypeptide complex further comprises at least 4, at least 5, least 6, at least 7, at least 8, at least 9, at least 10, least 11, at least 12, at least 13, at least 14, at least 15, or at least 16 third fusion polypeptides.

In some embodiments, the self-assembled polypeptide complex comprises a ratio of approximately 1:1, 1:2, 1:3, or 1:4 of first fusion polypeptides to all other fusion polypeptides.

In some embodiments, each fusion polypeptide within the self-assembled polypeptide complex comprises a ferritin light chain or a subunit of a ferritin light chain. In these embodiments, the self-assembled polypeptide complex does not comprise any ferritin heavy chains, subunits of ferritin heavy chains, or other ferritin components capable of binding to iron.

Also described herein are compositions comprising the nanocage, such as therapeutic or prophylactic compositions. Related methods and uses for treating and/or preventing COVID-19 are also described, wherein the method or use comprises administering the nanocage or composition described herein to a subject in need thereof.

Also described herein are nucleic acid molecules encoding the fusion proteins and polypeptides described herein, as well as vectors comprising the nucleic acid molecules and host cells comprising the vectors.

Polynucleotides encoding the fusion proteins described herein include polynucleotides with nucleic acid sequences that are substantially the same as the nucleic acid sequences of the polynucleotides of the present invention. “Substantially the same” nucleic acid sequence is defined herein as a sequence with at least 70%, at least 75%, at least 80%, at least 85%, at least 90%, at least 91%, at least 92%, at least 93%, at least 94%, at least 95% identity to another nucleic acid sequence when the two sequences are optimally aligned (with appropriate nucleotide insertions or deletions) and compared to determine exact matches of nucleotides between the two sequences.

Suitable sources of polynucleotides that encode fragments of antibodies include any cell, such as hybridomas and spleen cells, that express the full-length antibody. The fragments may be used by themselves as antibody equivalents, or may be recombined into equivalents, as described above. The DNA deletions and recombinations described in this section may be carried out by known methods, such as those described in the published patent applications listed above in the section entitled “Functional Equivalents of Antibodies” and/or other standard recombinant DNA techniques, such as those described below.

Another source of DNAs are single chain antibodies produced from a phage display library, as is known in the art.

Additionally, expression vectors are provided containing the polynucleotide sequences previously described operably linked to an expression sequence, a promoter and an enhancer sequence. A variety of expression vectors for the efficient synthesis of antibody polypeptide in prokaryotic, such as bacteria and eukaryotic systems, including but not limited to yeast and mammalian cell culture systems have been developed. The vectors of the present invention can comprise segments of chromosomal, non-chromosomal and synthetic DNA sequences.

Any suitable expression vector can be used. For example, prokaryotic cloning vectors include plasmids from E. coli, such as colEI, pCRI, pBR322, pMB9, pUC, pKSM, and RP4. Prokaryotic vectors also include derivatives of phage DNA such as M13 and other filamentous single-stranded DNA phages. An example of a vector useful in yeast is the 2p plasmid. Suitable vectors for expression in mammalian cells include well-known derivatives of SV-40, adenovirus, retrovirus-derived DNA sequences and shuttle vectors derived from combination of functional mammalian vectors, such as those described above, and functional plasmids and phage DNA.

Additional eukaryotic expression vectors are known in the art (e.g., P J. Southern & P. Berg, J. Mol. Appl. Genet, 1:327-341 (1982); Subramani et al, Mol. Cell. Biol, 1: 854-864 (1981); Kaufinann & Sharp, “Amplification And Expression of Sequences Cotransfected with a Modular Dihydrofolate Reductase Complementary DNA Gene,” J. Mol. Biol, 159:601-621 (1982); Kaufhiann & Sharp, Mol. Cell. Biol, 159:601-664 (1982); Scahill et al., “Expression And Characterization Of The Product Of A Human Immune Interferon DNA Gene In Chinese Hamster Ovary Cells,” Proc. Nat'l Acad. Sci USA, 80:4654-4659 (1983); Urlaub & Chasin, Proc. Nat'l Acad. Sci USA, 77:4216-4220, (1980), all of which are incorporated by reference herein).

The expression vectors typically contain at least one expression control sequence that is operatively linked to the DNA sequence or fragment to be expressed. The control sequence is inserted in the vector in order to control and to regulate the expression of the cloned DNA sequence. Examples of useful expression control sequences are the lac system, the trp system, the tac system, the trc system, major operator and promoter regions of phage lambda, the control region of fd coat protein, the glycolytic promoters of yeast, e.g., the promoter for 3-phosphoglycerate kinase, the promoters of yeast acid phosphatase, e.g., Pho5, the promoters of the yeast alpha-mating factors, and promoters derived from polyoma, adenovirus, retrovirus, and simian virus, e.g., the early and late promoters or SV40, and other sequences known to control the expression of genes of prokaryotic or eukaryotic cells and their viruses or combinations thereof.

Also described herein are recombinant host cells containing the expression vectors previously described. The fusion proteins described herein can be expressed in cell lines other than in hybridomas. Nucleic acids, which comprise a sequence encoding a polypeptide according to the invention, can be used for transformation of a suitable mammalian host cell.

Cell lines of particular preference are selected based on high level of expression, constitutive expression of protein of interest and minimal contamination from host proteins. Mammalian cell lines available as hosts for expression are well known in the art and include many immortalized cell lines, such as but not limited to, HEK 293 cells, Chinese Hamster Ovary (CHO) cells, Baby Hamster Kidney (BHK) cells and many others. Suitable additional eukaryotic cells include yeast and other fungi. Useful prokaryotic hosts include, for example, E. coli, such as E. coli SG-936, E. coli HB 101, E. coli W3110, E. coli X1776, E. coli X2282, E. coli DHI, and E. coli MRC1, Pseudomonas, Bacillus, such as Bacillus subtilis, and Streptomyces.

These present recombinant host cells can be used to produce fusion proteins by culturing the cells under conditions permitting expression of the polypeptide and purifying the polypeptide from the host cell or medium surrounding the host cell. Targeting of the expressed polypeptide for secretion in the recombinant host cells can be facilitated by inserting a signal or secretory leader peptide-encoding sequence (See, Shokri et al, (2003) Appl Microbiol Biotechnol. 60(6): 654-664, Nielsen et al, Prot. Eng., 10:1-6 (1997); von Heinje et al., Nucl. Acids Res., 14:4683-4690 (1986), all of which are incorporated by reference herein) at the 5′ end of the antibody-encoding gene of interest. These secretory leader peptide elements can be derived from either prokaryotic or eukaryotic sequences. Accordingly suitably, secretory leader peptides are used, being amino acids joined to the N-terminal end of a polypeptide to direct movement of the polypeptide out of the host cell cytosol and secretion into the medium.

The fusion proteins described herein can be fused to additional amino acid residues. Such amino acid residues can be a peptide tag to facilitate isolation, for example. Other amino acid residues for homing of the antibodies to specific organs or tissues are also contemplated.

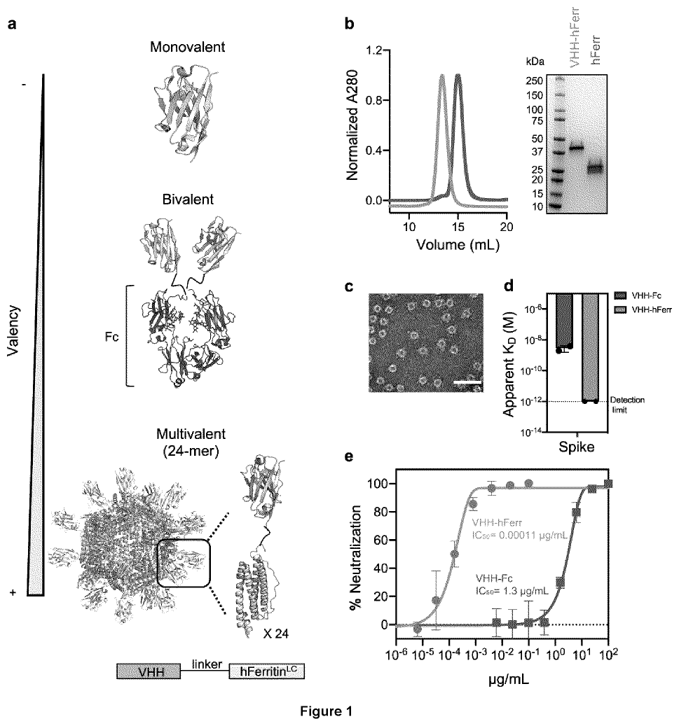

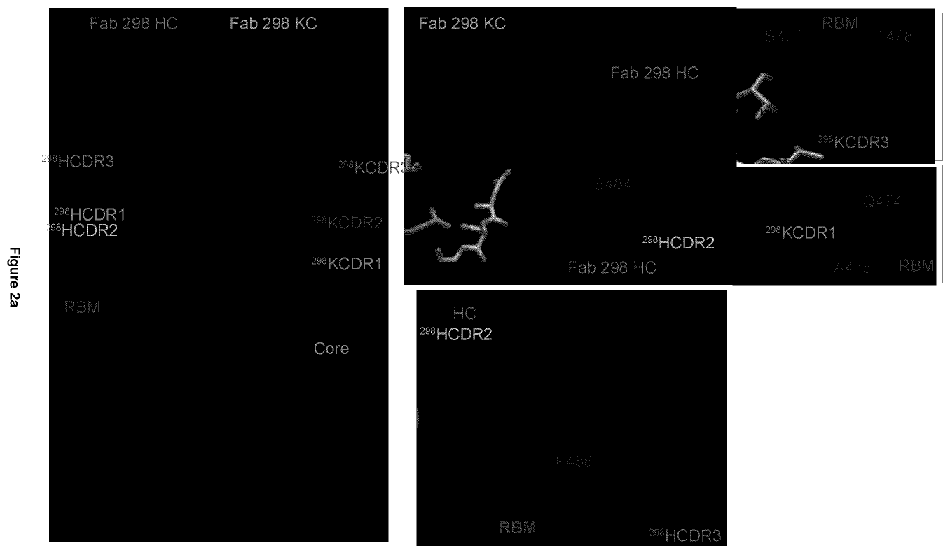

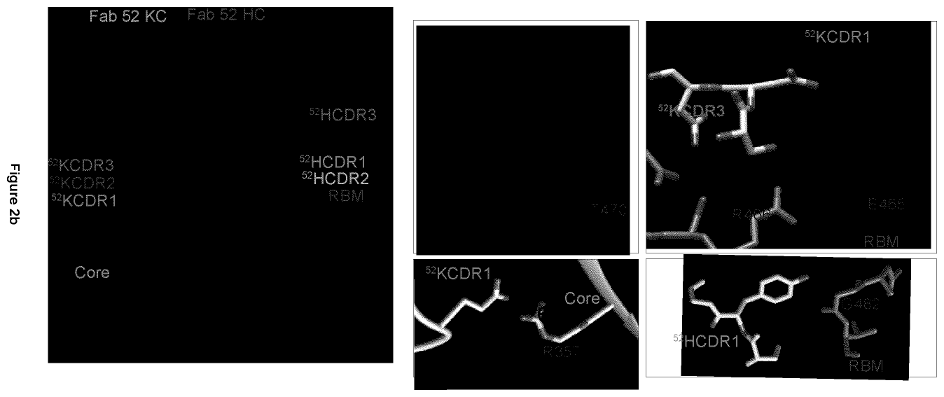

It will be understood that a Fab-nanocage can be generated by co-transfection of HC-ferritin and LC. Alternatively, single-chain Fab-ferritin nanocages can be used that only require transfection of one plasmid, as shown in FIG. 1C. This can be done with linkers of different lengths between the LC and HC for example 60 or 70 amino acids. When single-chain Fabs are used, it can be ensured that the heavy chain and light chain are paired. Tags (e.g. Flag, HA, myc, His6×, Strep, etc.) can also be added at the N terminus of the construct or within the linker for ease of purification as described above. Further, a tag system can be used to make sure many different Fabs are present on the same nanoparticle using serial/additive affinity chromatography steps when different Fab-nanoparticle plasmids are co-transfected. This provides multi-specificity to the nanoparticles. Protease sites (e.g. TEV, 3C, etc.) can be inserted to cleave linkers and tags after expression and/or purification, if desired.

Any suitable method or route can be used to administer the fusion proteins described herein. Routes of administration include, for example, oral, intravenous, intraperitoneal, subcutaneous, or intramuscular administration.

It is understood that the fusion proteins described herein, where used in a mammal for the purpose of prophylaxis or treatment, will be administered in the form of a composition additionally comprising a pharmaceutically acceptable carrier. Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the binding proteins. The compositions of the injection may, as is well known in the art, be formulated so as to provide quick, sustained or delayed release of the active ingredient after administration to the mammal.

Although human antibodies are particularly useful for administration to humans, they may be administered to other mammals as well. The term “mammal” as used herein is intended to include, but is not limited to, humans, laboratory animals, domestic pets and farm animals.

In one aspect, provided are methods that may be useful for treating, ameliorating, or preventing a SARS-CoV-2-related condition, generally comprising a step of administering a composition comprising a self-assembled polypeptide complex of the present disclosure to a subject.

A “SARS-CoV-2-related condition” refers to a condition (e.g., symptom or sign) that is associated with infection with SARS-CoV-2. In some embodiments, the condition is a level of SARS-CoV-2 RNA, protein, or viral particles in sample from a subject (e.g., the subject who is administered a self-assembled polypeptide complex as disclosed herein), which level is indicative of SARS-CoV-2 infection (e.g., because the level satisfies a threshold or exceeds a reference level indicative of SARS-CoV-2 infection). In some embodiments, the condition is a symptom associated with COVID-19 disease, e.g., fever, cough, tiredness, shortness of breath or difficulty breathing, muscle aches, chills, sore throat, runny nose, headache, chest pain, conjunctivitis, nausea, vomiting, diarrhea, loss of smell, loss of taste, or stroke). In some embodiments, the condition is associated with downstream sequelae of COVID-19 disease and/or is a symptom of long-term COVID-19 disease.

In some embodiments, the subject is a mammal, e.g., a human.

Compositions for administration to subjects generally comprise a self-assembled polypeptide complex as disclosed herein. In some embodiments, such compositions further comprise a pharmaceutically acceptable excipient.

Compositions may be formulated for administration for any of a variety of routes of administration, including systemic routes (e.g., oral, intravenous, intraperitoneal, subcutaneous, or intramuscular administration).

The above disclosure generally describes the present invention. A more complete understanding can be obtained by reference to the following specific examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The following examples do not include detailed descriptions of conventional methods, such as those employed in the construction of vectors and plasmids, the insertion of genes encoding polypeptides into such vectors and plasmids, or the introduction of plasmids into host cells. Such methods are well known to those of ordinary skill in the art and are described in numerous publications including Sambrook, J., Fritsch, E. F. and Maniatis, T. (1989), Molecular Cloning: A Laboratory Manual, 2nd edition, Cold Spring Harbor Laboratory Press, which is incorporated by reference herein.

Without further description, it is believed that one of ordinary skill in the art can, using the preceding description and the following illustrative examples, make and utilize the compounds of the present invention and practice the claimed methods. The following working examples therefore, specifically point out the typical aspects of the present invention, and are not to be construed as limiting in any way the remainder of the disclosure.

### EXAMPLES

### Example 1: Multivalency Transforms SARS-CoV-2 Antibodies into Ultrapotent Neutralizers

This example describes the design, expression, purification, and characterization of fusion proteins with apoferritin. Apoferritin protomers self-assemble into an octahedrally symmetric structure with an ˜6 nm hydrodynamic radius (Rh) composed of 24 identical polypeptides. The N-terminus of each apoferritin subunit points outwards of the spherical nanocage and is therefore accessible for the genetic fusion of proteins of interest. The fusion proteins were designed such that upon folding, apoferritin protomers act as building blocks that drive the multimerization of the 24 proteins fused to the apoferritin termini.

### Abstract

SARS-CoV-2, the virus responsible for COVID-19, has caused a global pandemic. Antibodies can be powerful biotherapeutics to fight viral infections. Here, we use the human apoferritin protomer as a modular subunit to drive oligomerization of antibody fragments and transform antibodies targeting SARS-CoV-2 into exceptionally potent neutralizers. Using this platform, half-maximal inhibitory concentration (IC₅₀) values as low as 9×10⁻¹⁴ M are achieved as a result of up to 10,000-fold potency enhancements compared to corresponding IgGs. Combination of three different antibody specificities and the fragment crystallizable (Fc) domain on a single multivalent molecule conferred the ability to overcome viral sequence variability together with outstanding potency and IgG-like bioavailability. The MULTi-specific, multi-Affinity antiBODY (Multabody or MB) platform thus uniquely leverages binding avidity together with multi-specificity to deliver ultrapotent and broad neutralizers against SARS-CoV-2. The modularity of the platform also makes it relevant for rapid evaluation against other infectious diseases of global health importance. Neutralizing antibodies are a promising therapeutic for SARS-CoV-2.

### Introduction

The continuous threat to public health from respiratory viruses such as the novel SARS-CoV-2 underscores the urgent need to rapidly develop and deploy prophylactic and therapeutic interventions to combat pandemics. Monoclonal antibodies (mAbs) have been used effectively for the treatment of infectious diseases as exemplified by palivizumab for the prevention of respiratory syncytial virus in high-risk infants¹ or Zmapp, mAb114, and REGN-EB3 for the treatment of Ebola². Consequently, mAbs targeting the Spike (S) protein of SARS-CoV-2 have been a focus for the development of biomedical countermeasures against COVID-19. To date, several antibodies targeting the S protein have been identified^{34,5,6,7,8,9,10,11,12,13,14,15,16,17,18,19} with bamlanivimab being the first antibody approved in the United States by the Food and Drug Administration (FDA) for the emergency treatment of SARS-CoV-2 in November 2020. Receptor binding domain (RBD)-directed mAbs that interfere with binding to angiotensin converting enzyme 2 (ACE2), the receptor for cell entry²⁰, are usually associated with the highest neutralization potencies^{6,18,19}.

mAbs can be isolated by B-cell sorting from infected donors, immunized animals, or by identifying binders in preassembled libraries. Despite these methodologies being robust and reliable for the discovery of virus-specific mAbs, identification of the best antibody clone is usually associated with a time-cost penalty. In addition, RNA viruses have higher mutations rates than DNA viruses and such mutations can significantly alter the potency of neutralizing antibodies. Indeed, several studies have already shown a reduction in neutralization potency from convalescent serum and resistance of certain mAbs^{21,22,23} to the more recent B.1.1.7²⁴, B.1.351²⁵, and B.1.1.28^{26,27} variants of SARS-CoV-2. Hence, there is an unmet need for the development of a platform that bridges antibody discovery and the rapid identification and deployment of highly potent neutralizers less susceptible to viral sequence variability.

The potency of an antibody is greatly affected by its ability to simultaneously interact multiple times with its epitope^{28,29,30}. This enhanced apparent affinity, known as avidity, has been previously reported to increase the neutralization potency of nanobodies^{31,32} and of IgGs over Fabs^{8,10,16} against SARS-CoV-2. To leverage the full power of binding avidity, we have developed an antibody-scaffold technology using the human apoferritin protomer as a modular subunit to multimerize antibody fragments and propel mAbs into ultrapotent neutralizers against SARS-CoV-2. Indeed, the resulting Multabody molecules can increase potency by up to four orders of magnitude over corresponding IgGs. In addition, we demonstrate the ability of this technology to combine three different Fab specificities to better overcome point mutations in the Spike. The Multabody offers a versatile IgG-like “plug-and-play” platform to enhance antiviral characteristics of mAbs against SARS-CoV-2, and demonstrates the power of avidity as a mechanism to be leveraged against viral pathogens.

### Materials and Methods

### Protein Expression and Purification

Genes encoding VHH-human apoferritin fusion, Fc fusions, Fabs, IgG, and RBD mutants were synthesized and cloned by GeneArt (Life Technologies) into the pcDNA3.4 expression vector. All constructs were expressed transiently in HEK 293F cells (Thermo Fisher Scientific) at a density of 0.8×10⁶ cells/mL with 50 μg of DNA per 200 mL of cells using FectoPRO (Polyplus Transfections) in a 1:1 ratio unless specified otherwise. After 6-7 days of incubation at 125 rpm oscillation at 37° C., 8% CO₂, and 70% humidity in a Multitron Pro shaker (Infors HT), cell suspensions were harvested by centrifugation at 5000×g for 15 min and supernatants were filtered through a 0.22 μm Steritop filter (EMD Millipore). Fabs and IgGs were transiently expressed by co-transfecting 90 μg of the LC and the HC in a 1:2 ratio and purified using KappaSelect affinity column (GE Healthcare) and HiTrap Protein A HP column (GE Healthcare), respectively with 100 mM glycine pH 2.2 as the elution buffer. Eluted fractions were immediately neutralized with 1 M Tris-HCl, pH 9.0, and further purified using a Superdex 200 Increase size exclusion column (GE Healthcare). Fc fusions of ACE2 and VHH-72 were purified the same way as IgGs. The VHH-72 apoferritin fusion was purified by hydrophobic interaction chromatography using a HiTrap Phenyl HP column and the eluted fraction was loaded onto a Superose 6 10/300 GL size exclusion column (GE Heathcare) in 20 mM sodium phosphate pH 8.0, 150 mM NaCl. Wild type (BEI NR52309) and mutant RBDs, the prefusion S ectodomain (BEI NR52394) and Fc receptors (FcRn and FcγRI) from mouse and human were purified using a HisTrap Ni-NTA column (GE Healthcare). Ni-NTA purification was followed by Superose 6 in the case of the S trimer and Superdex 200 Increase size exclusion columns (GE Heathcare) in the case of the RBD and Fc receptors, in all cases in 20 mM phosphate pH 8.0, 150 mM NaCl buffer.

### Design, Expression and Purification of Multabodies

All molecules referred herein as Multabodies contain scFab and scFc fragments. The scFabs and scFc polypeptide constructs were generated using a 70 amino acid flexible linker [(GGGGS)ₓ₁₄] to generate heterodimers and homodimer fragments, respectively. Specifically, the C terminus of the Fab light chain is fused, through the linker, to the N terminus of the Fab heavy chain. In the case of the scFc, the two single Fc chains that form the functional homodimer Fc were fused in tandem. The individual domains are fused to apoferritin monomers with a 25 amino acid linker: (GGGGS)ₓ₅. Genes encoding scFab and scFc fragments linked to half apoferritin were generated by deletion of residues 1 to 90 (C-Ferritin) and 91 to 175 (N-Ferritin) of the light chain of human apoferritin. Transient transfection of the Multabodies in HEK 293F cells were obtained by mixing 66 μg of the plasmids scFab-human apoferritin:scFc-human N-Ferritin:scFab-C-Ferritin in a 2:1:1 ratio. Addition of scFab-human apoferritin allowed efficient Multabody assembly and increased the number of Fab's compared to Fc's in the final molecule, thus favoring Fab avidity over Fc avidity. In the case of multi-specific Multabodies, a 4:2:1:1 ratio of scFab1-human apoferritin:scFc-human N-Ferritin:scFab2-C-Ferritin:scFab3-C-Ferritin was used. The DNA mixture was filtered and incubated at room temperature (RT) with 66 μl of FectoPRO before adding to the cell culture. Split Multabodies were purified by affinity chromatography using a HiTrap Protein A HP column (GE Healthcare) with 20 mM Tris pH 8.0, 3 M MgCl₂ and 10% glycerol elution buffer. Fractions containing the protein were concentrated and further purified by gel filtration on a Superose 6 10/300 GL column (GE Healthcare).

### Negative-Stain Electron Microscopy

Three microliters of Multabody at a concentration approximately of 0.02 mg/mL was placed on the surface of a carbon-coated copper grid that had previously been glow-discharged in air for 15 s, allowed to adsorb for 30 s, and stained with 3 μL of 2% uranyl formate. Excess stain was removed immediately from the grid using Whatman No. 1 filter paper and an additional 3 μL of 2% uranyl formate was added for 20 s. Grids were imaged with a FEI Tecnai T20 electron microscope operating at 200 kV and equipped with an Orius charge-coupled device (CCD) camera (Gatan Inc).

### Biolayer Interferometry

Direct binding kinetics measurements were conducted using an Octet RED96 BLI system (Sartorius ForteBio) in PBS pH 7.4, 0.01% BSA, and 0.002% Tween at 25° C. His-tagged RBD, SARS-CoV-2 Spike was loaded onto Ni-NTA (NTA) biosensors (Sartorius ForteBio) to reach a BLI signal response of 0.8 nm. Association rates were measured by transferring the loaded biosensors to wells containing a two-fold dilution series from 250 to 8 nM (Fabs), 125 to 4 nM (IgG), and 16 to 0.5 nM (MB). Dissociation rates were measured by dipping the biosensors into buffer-containing wells. The duration of each step was 180 s. Fc characterization in the split Multabody design was assessed by measuring binding to hFcγRI and hFcRn loaded onto Ni-NTA (NTA) biosensors following the experimental conditions and concentration ranges indicated above. To probe the theoretical capacity of the Multabodies to undergo endosomal recycling, binding to the hFcRn p2-microglobulin complex was measured at physiological (7.4) and endosomal (5.6) pH. Similarly, Fc characterization of the mouse surrogate MB was assessed by measuring binding to mFcγRI and mFcRn, pre-immobilized onto Ni-NTA (NTA) biosensors. Two-fold dilution series from 100 to 3 nM (IgG) and 10 to 0.3 nM (MB) were used. Analysis of the sensograms was performed using the Octet software, with a 1:1 fit model. Competition assays were performed in a two-step binding process. Ni-NTA biosensors preloaded with His-tagged RBD were first dipped into wells containing the primary antibody at 50 μg/mL for 180 s. After a 30 s baseline period, the sensors were dipped into wells containing the second antibody at 50 μg/ml for an additional 300 s. All incubation steps were performed in PBS pH 7.4, 0.01% BSA, and 0.002% Tween at 25° C. ACE2-Fc was used to map mAb binding to the receptor binding site.

### Dynamic Light Scattering

The Rh of the Multabody was determined by dynamic light scattering (DLS) using a DynaPro Plate Reader III (Wyatt Technology). About 20 μL of the Multabody at a concentration of 1 mg/mL was added to a 384-well black, clear bottom plate (Corning) and measured at a fixed temperature of 25° C. with a duration of 5 s per read. Particle size determination and polydispersity were obtained from the accumulation of five reads using the Dynamics software (Wyatt Technology).

### Aggregation Temperature

Aggregation temperature (T_{agg}) of the Multabodies and parental IgGs were determined using a UNit instrument (Unchained Labs). Samples were concentrated to 1.0 mg/mL and subjected to a thermal ramp from 25 to 95° C. with 1° C. increments. T_{agg} was determined as the temperature at which 50% increase in the static light scattering at a 266 nm wavelength relative to baseline was observed (i.e., the maximum value of the differential curve). The average and the standard error of two independent measurements were calculated using the UNit analysis software.

### Pharmacokinetics and Immunogenicity

A surrogate Multabody composed of the scFab and scFc fragments of mouse HD37 (anti-hCD19) IgG2a fused to the N-terminus of the light chain of mouse apoferritin (mFerritin) was used for the study. HD37 scFab-mFerritin:Fc-mFerritin:mFerritin in a 2:1:1 ratio was transfected and purified following the procedure described above. L234A, L235A, and P329G (LALAP) mutations were introduced in the mouse IgG2a Fc-construct to silence effector functions of the Multabody⁴⁸. In vivo studies were performed using 12-week-old male C57BL/6 mice purchased from Charles River (Strain code: 027), housed in individually-vented cages under 12 h light/dark cycle (7 a.m./7 p.m.) at a temperature of 21-23° C. and a humidity of 40-55%. All procedures were approved by the Local Animal Care Committee at the University of Toronto Scarborough. A single injection of ˜5 mg/kg of Multabodies or control samples (HD37 single chain IgG-IgG1 or IgG2a subtypes) and Helicobacter pylori ferritin (HpFerritin)-PfCSP malaria peptide in 200 μL of PBS (pH 7.5) were subcutaneously injected. Blood samples were collected at multiple time points and serum samples were assessed for levels of circulating antibodies and anti-drug antibodies by ELISA. Briefly, 96-well Pierce Nickel Coated Plates (Thermo Fisher) were coated with 50 μL at 0.5 μg/ml of the His₆ₓ-tagged antigen hCD19 to determine circulating HD37-specific concentrations using reagent-specific standard curves for IgGs and Multabodies. HRP-ProteinA (Invitrogen) was used to detect the levels of IgG/MBs bound (dilution 1:10,000). For anti-drug-antibody determination, Nunc MaxiSorp plates (Biolegend) were coated with a 12-mer HD37 scFab-mFerritin or with the HpFerritin-PfCSP malaria peptide. 1:100 sera dilution was incubated for 1 h at RT and further develop using HRP-ProteinA (Invitrogen) as a secondary molecule (dilution 1:10,000). The chemiluminescence signal at 450 nm was quantified using a Synergy Neo2 Multi-Mode Assay Microplate Reader (Biotek Instruments).

### Biodistribution

Eight-week-old male BALB/c mice were purchased from The Jackson Laboratory and housed in individually-vented caging. Mice were housed 14 h of light/10 h dark with phased in dawn to dusk intensity, maximum at noon at a temperature of 20-21° C. and a humidity of 40-60%. All procedures were approved by the Local Animal Care Committee at the University of Toronto. Multabodies composed of the scFab and scFc fragments of mouse HD37 IgG2a fused to the N-terminus of mouse apoferritin light chain was used for this study. HD37 IgG2a Multabody or control samples (HD37 single chain IgG2a) were fluorescently conjugated with Alexa-647 using Alexa Fluor™ 647 Antibody Labeling kit (Invitrogen) as per the manufacturer's instruction. The 15 nm gold nanoparticles labeled with Alexa Fluor™ 647 were purchased from Creative Diagnostics (GFLV-15). PerkinElmer IVIS Spectrum (PerkinElmer) was used to conduct noninvasive biodistribution experiments. BALB/c mice were injected subcutaneously into the loose skin over the shoulders with ˜5 mg/kg of the MB, HD37 IgG2a, or gold nanoparticles in 200 μL of PBS (pH 7.5) and imaged at time 0, 1 h, 6 h, 24 h, 2, 3, 4, 8, and 11 days following injection. Prior to imaging, mice were placed in an anesthesia induction chamber containing a mixture of isoflurane and oxygen for 1 min. Anesthetized mice were then placed in the prone position at the center of a built-in heated docking system within the IVIS imaging system (maintained at 37° C. and supplied with a mixture of isoflurane and oxygen). For whole body 2D imaging, mice were imaged for 1-2 s (excitation 640 nm and emission 680 nm) inside the imaging system. Data were analyzed using the IVIS software (Living Image Software for IVIS). After confirming the fluorescent signal from 2D epi-illumination images, 3D transilluminating fluorescence imaging tomography (FLIT) was performed on regions of interest using a built-in scan field of 3×3 or 3×4 transillumination positions. A series of 2D fluorescent surface radiance images were taken at various transillumination positions using an excitation of 640 and 680 nm emission. A series of CT scans were also taken at the corresponding positions. A 3D distribution map of the fluorescent signal was reconstructed by combining fluorescent signal and CT scans. Resulting 3D fluorescent images were thresholded based on the 3D images of PBS injected mice taken at the corresponding body positions. Images were mapped to the rainbow LUT in the IVIS software, with the upper end of the color scale set to 50 pmol M⁻¹ cm⁻¹ for mice injected with gold nanoparticles, and 1000 pmol M⁻¹ cm⁻¹ for MB and IgG2a injected mice, to allow for better visualization of biodistribution over the time course. A mouse organ registration feature of the IVIS software was used as a general guideline for assessing the sample body locations from 3D images.

Panning of Phage libraries against the RBD of SARS-CoV-2 The commercial SuperHuman 2.0 Phage library (Distributed Bio/Charles River Laboratories) was used to identify monoclonal antibody binders to the SARS-CoV-2 RBD. For this purpose, an RBD-Fc-Avi tag construct of the SARS-CoV-2 was expressed in the EXPi-293 mammalian expression system. This protein was subsequently purified by protein G Dynabeads, biotinylated and quality-controlled for biotinylation and binding to ACE2 recombinant protein (Sino Biologics Inc). The SuperHuman 2.0 Phage library (5×10¹²) was heated for 10 min at 72° C. and de-selected against Protein G Dynabeads™ (Invitrogen), M-280 Streptavidin Dynabeads™ (Invitrogen), Histone from Calf Thymus (Sigma), Human IgG (Sigma) and ssDNA-Biotin NNK from Integrated DNA Technologies and DNA-Biotin NNK from Integrated DNA Technologies. Next, the library was panned against the RBD-captured by M-280 Streptavidin Dynabeads™ using an automated protocol on Kingfisher FLEX (Thermofisher). Selected phages were acid eluted from the beads and neutralized using Tris-HCl pH 7.9 (Teknova). ER2738 cells were infected with the neutralized phage pools at OD₆₀₀=0.5 at a 1:10 ratio and after 40 min incubation at 37° C. and 100 rpm, the phage pools were centrifuged and incubated on agar with antibiotic selection overnight at 30° C. The rescued phages were precipitated by PEG and subjected to three additional rounds of soluble-phase automated panning. PBST/1% BSA buffer and/or PBS/1% BSA was used in the de-selection, washes and selection rounds.

Screening of Anti-SARS-CoV-2 scFvs in Bacterial PPE with SARS-CoV-2 RBD

Anti-SARS-CoV-2 RBD scFvs selected from phage display were expressed and screened using high-throughput surface plasmon resonance (SPR) on Carterra LSA Array SPR instrument (Carterra) equipped with HC200M sensor chip (Carterra) at 25° C. A V5 epitope tag was added to the scFv to enable capture via immobilized anti-V5 antibody (Abcam, Cambridge, MA) that was pre-immobilized on the chip surface by standard amine-coupling. Briefly: the chip surface was first activated by 10 min injection of a 1:1:1 (v/v/v) mixture of 0.4 M 1-ethyl-3-(3-dimethylaminopropyl) carbodiimide hydrochloride (EDC), 0.1 M N-hydroxysulfosuccinimide (sNHS) and 0.1 M 2-(N-morpholino) ethanesulfonic acid (MES) pH 5.5. Then, 50 μg/ml of anti-V5 tag antibody prepared in 10 mM sodium acetate pH 4.3 was coupled for 14 min and the excess reactive esters were blocked with 1 M ethanolamine HCl pH 8.5 during a 10 min injection. For screening, a 384-ligand array comprising of crude bacterial periplasmic extracts (PPE) containing the scFvs (one spot per scFv) was prepared. Each extract was prepared at a twofold dilution in running buffer (10 mM HEPES pH 7.4, 150 mM NaCl, 3 mM EDTA, and 0.01% (v/v) Tween-20 (HBSTE)) and printed on the anti-V5 surface for 15 min. SARS-CoV-2 RBD Avi Tev His tagged was then prepared at 0, 3.7, 11.1, 33.3, 100, 37, and 300 nM in 10 mM HEPES pH 7.4, 150 mM NaCl, and 0.01% (v/v) Tween-20 (HBST) supplemented with 0.5 mg/ml BSA and injected as analyte for 5 min with a 15 min dissociation time. Samples were injected in ascending concentration without any regeneration step. Binding data from the local reference spots was used to subtracted signal from the active spots and the nearest buffer blank analyte responses were subtracted to double-reference the data. The double-referenced data were fitted to a simple 1:1 Langmuir binding model in Carterra's Kinetic Inspection Tool (version October 2019). Twenty medium-affinity binders from phage display screening were selected for the present study.

### Pseudovirus Production and Neutralization

SARS-CoV-2 pseudotyped viruses (PsV) were generated using an HIV-based lentiviral system⁴⁹ with few modifications. Briefly, 293T cells were co-transfected with a lentiviral backbone encoding the luciferase reporter gene (BEI NR52516), a plasmid expressing the Spike (BEI NR52310) and plasmids encoding the HIV structural and regulatory proteins Tat (BEI NR52518), Gag-pol (BEI NR52517), and Rev (BEI NR52519) using BioT transfection reagent (Bioland Scientific) and following the manufacturer's instructions. 24 h post transfection at 37° C., 5 mM sodium butyrate was added to the media and the cells were incubated for an additional 24-30 h at 30° C. SARS-CoV-2 Spike mutant D614G was kindly provided by D. R. Burton (The Scripps Research Institute), SARS-COV-2 PsV variant B.1.351 was kindly provided by D. D. Ho (Columbia University) and the rest of the PsV mutants were generated using the KOD-Plus mutagenesis kit (Toyobo, Osaka, Japan) using primers described in Table 1. PsV particles were harvested, passed through 0.45 μm pore sterile filters and finally concentrated using a 100 K Amicon (Merck Millipore Amicon-Ultra 2.0 Centrifugal Filter Units).

TABLE 1
Primer Sequences

| Primer name | Primer |
|---|---|
| N2340_fwd | CAGATCACCCGGTTTCAGACACTGCTGGCC |
| N234Q_rev | GATGCCGATGGGCAGATCCACCAGGGG |
| L452R_fwd | CGGTACCGGCTGTTCCGGAAGTCCAATCTG |
| L452_rev | GTAATTGTAGTTGCCGCCGACTTTGG |
| A475V_fwd | GTGGGCAGCACCCCTTGTAACGGCGTGGAAG |
| A475V_rev | CTGATAGATCTCGGTGGAGATGTCOC |
| V483A_fwd | GCCGAAGGCTTCAACTGCTACTTOCCACTGC |
| V483A_rev | GCCGTTACAAGGGGTGCTGCCGGCC |
| N439K_fwd | AAGAACCTGGACTCCAAAGTCGGCGGCAACTAC |
| N439K_rev | GCTGTTCCAGGCAATCACACAGCCGGTG |

Neutralization was determined in a single-cycle neutralization assay using 293T-ACE2 cells (BEI NR52511) and HeLa-ACE2 cells (kindly provided by D. R. Burton; The Scripps Research Institute). Cells were seeded the day before the experiment at a density of 10,000 cells/well in a 100 μl volume. In the case of 293T cells, plates where pre-coated with poly-L-lysine (Sigma-Aldrich). The day of the experiment, 50 μl of serially diluted IgGs and MB samples were incubated with 50 μl of PsV for 1 h at 37° C. After 1 h incubation, the incubated volume was added to the cells and incubated for 48 h. PsV neutralization was monitored by adding 50 μl Britelite plus reagent (PerkinElmer) to 50 μl of the cells and after 2 min incubation, the volume was transferred to a 96-well white plate (Sigma-Aldrich) and the luminescence in relative light units (RLUs) was measured using a Synergy Neo2 Multi-Mode Assay Microplate Reader (Biotek Instruments). Two to three biological replicates with two technical replicates each were performed. IC₅₀ fold increase was calculated as:

IgG IC₅₀ (μg/mL)/MB IC₅₀ (μg/mL)

### Authentic Virus Neutralization

VeroE6 cells were seeded in a 96 F plate at a concentration of 30,000/well in DMEM supplemented with 100 U Penicillin, 100 U Streptomycin, and 10% FBS. Cells were allowed to adhere to the plate and rest overnight. After 24 h, fivefold serial dilutions of the IgG and MB samples were prepared in DMEM supplemented with 100 U Penicillin and 100 U Streptomycin in a 96 R plate in quadruplicates (25 μL/well). About 25 μL of SARS-CoV-2/SB2-P4-PB⁵⁰ Clone 1 was added to each well at 100 TCID/well and incubated for 1 h at 37° C. with shaking every 15 min. After co-culturing, the media from the VeroE6 plate was removed, and 50 μL antibody-virus sample was used to inoculate VeroE6 cells in quadruplicates for 1 h at 37° C., 5% CO₂, shaking every 15 min. After 1 h inoculation, the inoculum was removed and 200 μL of fresh DMEM supplemented with 100 U Penicillin, 100 U Streptomycin, and 2% FBS was added to each well. The plates were further incubated for 5 days. The cytopathic effect (CPE) was monitored and PRISM was used to calculate IC₅₀ values. Three biological replicates with four technical replicates each were performed.

Cross-Linking of Spike Protein with Fabs 80, 298 and 324

About 100 μg of Spike trimer was mixed with 2× molar excess of Fab 80, 298, or 324 in 20 mM HEPES pH 7.0 and 150 mM NaCl. Proteins were crosslinked by addition of 0.075% (v/v) glutaraldehyde (Sigma Aldrich) and incubated at RT for 120 min. Complexes were purified via size exclusion chromatography (Superose6 Increase 10/300 GL, GE Healthcare), concentrated to 0.5 mg/mL and directly used for cryo-EM grid preparation.

### Cross-Linking of Fab 46-RBD Complex

About 100 μg of Fab 46 was mixed with 2× molar excess of RBD in 20 mM HEPES pH 7.0 and 150 mM NaCl. The complex was crosslinked by addition of 0.05% (v/v) glutaraldehyde (Sigma Aldrich) and incubated at RT for 45 min. The cross-linked complex was purified via size exclusion chromatography (Superdex 200 Increase 10/300 GL, GE Healthcare), concentrated to 2.0 mg/ml and directly used for cryo-EM grid preparation.

### Cryo-EM Data Collection and Image Processing

Three microliters of sample was deposited on holey gold grids prepared in-house⁵¹, which were glow-discharged in air for 15 s with a PELCO easiGlow (Ted Pella) before use. Sample was blotted for 6 s with a modified FEI Mark III Vitrobot (maintained at 4° C. and 100% humidity) using an offset of ˜5, and subsequently plunge-frozen in a mixture of liquid ethane and propane. Data were acquired at 300 kV with a Thermo Fisher Scientific Titan Krios G3 electron microscope and prototype Falcon 4 camera operating in electron counting mode at 250 frames/s. Movies were collected for 9.6 s with 29 exposure fractions, a camera exposure rate of ˜5 e⁻/pix/s, and total specimen exposure of ˜44 e⁻/Å². No objective aperture was used. The pixel size was calibrated at 1.03 Å/pixel from a gold diffraction standard. The microscope was automated with the EPU software package and data collection were monitored with cryoSPARC Live⁵².

To overcome preferred orientation encountered with some of the samples, tilted data collection was employed⁵³. For the Spike-Fab 80 complex, 820 0° tilted movies and 2790 400 tilted movies were collected. For the Spike-Fab 298 complex, 4259 0° tilted movies and 3513 40° tilted movies were collected. For the Spike-Fab 324 complex, 1098 0° tilted movies and 3380 40° tilted movies were collected. For the RBD-Fab 46 complex, 4722 0° tilted movies were collected. For 0° tilted movies, cryoSPARC patch motion correction was performed. For 40° tilted movies, Relion MotionCorr^{54,55} was used. Micrographs were then imported into cryoSPARC and patch CTF estimation was performed. Templates generated from 2D classification during the cryoSPARC Live session were used for template selection of particles. 2D classification was used to remove junk particle images, resulting in a dataset of 80,951 particle images for the Spike-Fab 80 complex, 203,138 particle images for the Spike-Fab 298 complex, 64,365 particle images for the Spike-Fab 324 complex, and 2,143,629 particle images for the RBD-Fab 46 complex. Multiple rounds of multi-class ab initio refinement were used to clean up the particle image stacks, and homogeneous refinement was used to obtain consensus structures. For tilted particles, particle polishing was done within Relion at this stage and reimported back into cryoSPARC. For the Spike-Fab complexes, extensive flexibility was observed. 3D variability analysis was performed⁵⁶ and together with heterogeneous refinement used to classify out the different states present. Nonuniform refinement was then performed on the final set of particle images⁵⁷. For the RBD-Fab 46 complex, cryoSPARC ab initio refinement with three classes was used iteratively to clean up the particle image stack. Thereafter, the particle image stack with refined Euler angles was brought into cisTEM for reconstruction⁵⁸ to produce a 4.0 Å resolution map. Transfer of data between Relion and cryoSPARC was done with pyem⁵⁹.

### Crystallization and Structure Determination

A ternary complex of 52 Fab-298 Fab-RBD was obtained by mixing 200 μg of RBD with 2× molar excess of each Fab in 20 mM Tris pH 8.0, 150 mM NaCl, and subsequently purified via size exclusion chromatography (Superdex 200 Increase 10/300 GL, GE Healthcare). Fractions containing the complex were concentrated to 7.3 mg/ml and mixed in a 1:1 ratio with 20% (w/v) 2-propanol, 20% (w/v) PEG 4000, and 0.1 M sodium citrate pH 5.6. Crystals appeared after ˜1 day and were cryoprotected in 10% (v/v) ethylene glycol before being flash-frozen in liquid nitrogen.

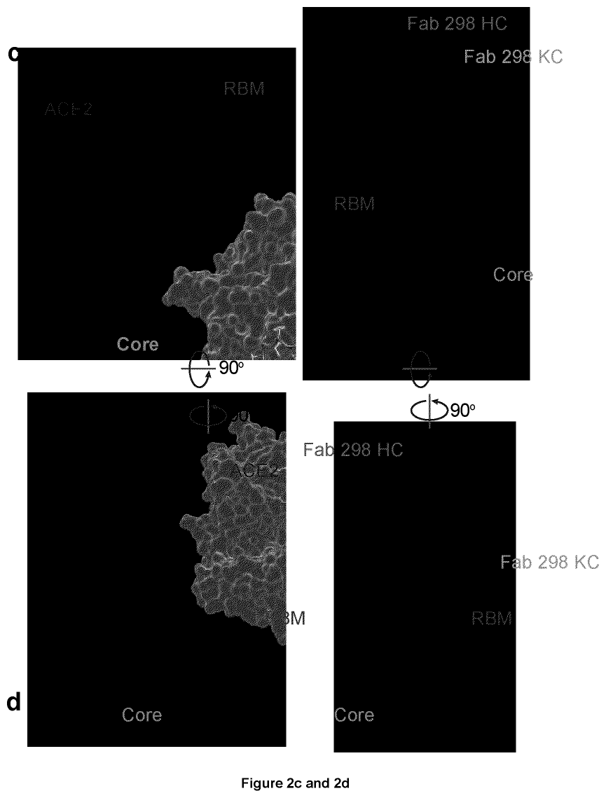

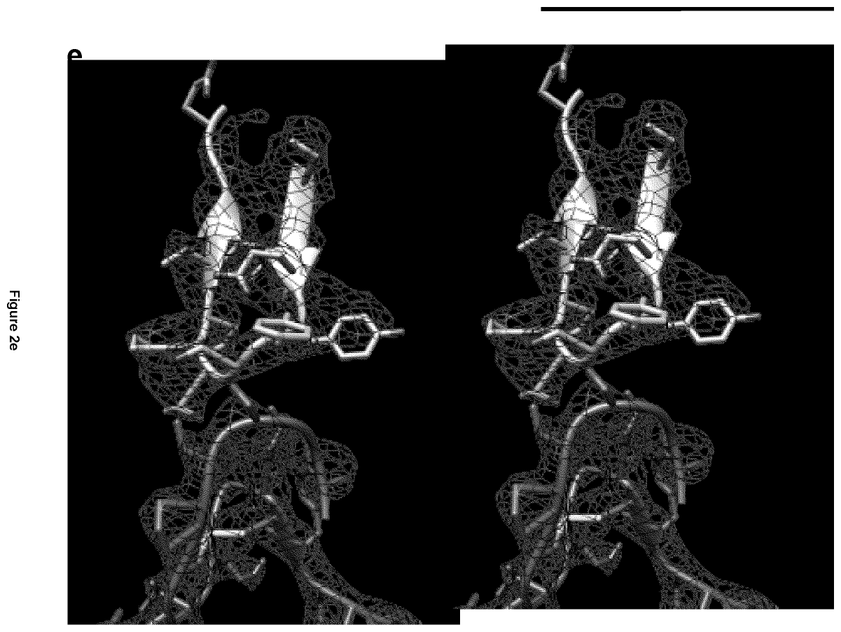

Data were collected on the 23-ID-D beamline at the Argonne National Laboratory Advanced Photon Source. The dataset was processed using XDS⁶⁰ and XPREP. Phases were determined by molecular replacement using Phaser⁶¹ with CNTO88 Fab as a model for 52 Fab (PDB ID: 4DN3), 20358 Fab as a model for 298 Fab (PDB ID: 5CZX), and PDB ID: 6XDG as a search model for the RBD. Refinement of the structure was performed using phenix.refine⁶² and iterations of manual building in Coot⁶³. PyMOL was utilized for structure analysis and figure rendering⁶⁴. Access to all software was supported through SBGrid⁶⁵. Representative electron density for the two Fab-RBD interfaces is shown in FIG. 2e, f.

### Materials Availability

The electron microscopy maps have been deposited in the Electron Microscopy Data Bank (EMDB) with accession codes EMD-22738, EMD-22739, EMD-22740, and EMD-22741 (Table 2). The crystal structure of the 298-52-RBD complex (Table 3) is available from the Protein Data Bank under accession PDB ID: 7K9Z. The sequences of the monoclonal antibodies used are provided with this paper (Table 4). Additional PDB/EMDB entries were used throughout the manuscript to perform a comparative analysis of the different epitope bins targeted by mAbs. The entries used in this analysis are: REGN10933 (PDB ID: 6XDG), CV30 (PDB ID: 6XE1), C105 (PDB ID: 6XCM), COVA2-04 (PDB ID: 7JMO), COVA2-39 (PDB ID: 7JMP), CC12.1 (PDB ID: 6XC2), BD23 (PDB ID: 7BYR), B38 (PDB ID: 7BZ5), P2C-1F11 (PDB ID: 7BWJ), 2-4 (PDB ID: 6XEY), CB6 (PDB ID: 7C01), REGN10987 (PDB ID: 6XDG), S309 (PDB ID: 6WPS, 6WPT), EY6A (PDB ID: 6ZCZ), CR3022 (PDB ID: 6YLA), H014 (PDB ID: 7CAH), 4-8 (EMDB ID: 22159), 4A8 (PDB ID: 7C2L), and 2-43 (EMDB ID: 22275).

TABLE 2
Cryo-EM data collection and image processing

| | Fab 80-Spike | Fab 298-Spike | Fab 324-Spike | Fab 46-RBD |
|---|---|---|---|---|
| EMDB ID | EMD-22739 | EMD-22740 | EMD-22741 | EMD-22738 |
| Data Collection | | | | |
| Electron | Titan Krios G3 | Titan Krios G3 | Titan Krios G3 | Titan Krios |
| microscope | | | | G3 |
| Camera | Falcon 4EC | Falcon 4EC | Falcon 4EC | Falcon 4EC |
| Voltage (kV) | 300 | 300 | 300 | 300 |
| Nominal | 75,000 | 75,000 | 75,000 | 75,000 |
| magnification | | | | |
| Calibrated | 1.03 | 1.03 | 1.03 | 1.03 |
| physical pixel | | | | |
| size (Å) | | | | |
| Total exposure | 44 | 44 | 44 | 44 |
| (e-/Å²) | | | | |
| Number of | 29 | 29 | 29 | 29 |
| frames | | | | |
| Image | | | | |
| Processing | | | | |
| Motion | cryoSPARC v2, | cryoSPARC v2, | cryoSPARC v2, | cryoSPARCv2 |
| correction | Relion | Relion | Relion | |
| software | MotionCorr | MotionCorr | MotionCorr | |
| CTF estimation | cryoSPARCv2 | cryoSPARCv2 | cryoSPARCv2 | cryoSPARCv2 |
| software | | | | |
| Particle | cryoSPARCv2 | cryoSPARCv2 | cryoSPARCv2 | cryoSPARCv2 |
| selection | | | | |
| software | | | | |
| 3D map | cryoSPARCv2 | cryoSPARCv2 | cryoSPARCv2 | cisTEM |
| classification | | | | |
| and refinement | | | | |
| software | | | | |
| Micrographs | 3610 | 7772 | 4478 | 4722 |
| used (total) | | | | |
| 0° tilt | 820 | 4259 | 1098 | 4722 |
| 40° tilt | 2790 | 3513 | 3380 | 0 |
| Global | 6.2 | 6.2 | 6 | 4 |
| resolution (Å) | | | | |
| Particles in final | 7,525 | 26,972 | 18,595 | 32,283 |
| maps | | | | |

TABLE 3
X-ray crystallography data collection and refinement statistics

| | Fab 52- Fab 298- |
|---|---|
| | SARS-CoV-2 RBD |
| PDB ID | 7K9Z |
| Data Collection | |
| Wavelength (Å) | 1.03317 |
| Space group | P 3₂ 2 1 |
| Cell dimensions | |
| a, b, c (Å) | 87.6, 87.6, 325.1 |
| α, β, γ (°) | 90.0, 90.0, 120.0 |
| Resolution (Å) | 39.66-2.95 (3.05-2.95) |
| No. Molecules in ASU | 1 |
| No. Total observations | 496,550 (43,958) |
| No unique observations | 31,545 (3,060) |
| Multiplicity | 15.7 (14.3) |
| R_{merge} (%) | 16.8 (74.2) |
| Rₚᵢₘ (%) | 4.3 (20.1) |
| <I/σI> | 12.3 (1.4) |
| CC_{1/2} | 99.8 (86.3) |
| Completeness (%) | 99.9 (99.9) |
| Refinement | |
| Non-hydrogen atoms | 8061 |
| Macromolecule | 8047 |
| Glycan | 14 |
| R_{work}/R_{free} | 0.259/0.286 |
| Rms deviations | |
| Bond lengths (Å) | 0.002 |
| Bond angles (°) | 0.53 |
| Ramachandran plot | |
| Favored regions (%) | 95.6 |
| Allowed regions (%) | 4.1 |
| Outliers (%) | 0.3 |
| Rotamer Outliers (%) | 2.6 |
| B-factors (Å²) | |
| Wilson B-factor | 78.6 |
| Average B-factors | 103.9 |
| Average macromolecule | 103.9 |
| Average glycan | 114.3 |

TABLE 4

| | | | | | | IC50 SARS- | | | |
|---|---|---|---|---|---|---|---|---|---|
| | | | | IC50 SARS- | | CoV2 PsV | | | |
| | | | RBD | CoV2/SB2 | | -P4-PB | | Variable | Variable |
| mAb | | | KD | (μg/mL) | | (μg/mL) | | Heavy Chain | Light Chain |
| ID | VH | VK | (nM) | MB | IgG | MB | IgG | sequence | sequence |
| 56 | IGHV1- | IGKV1- | 23 | >50 | >50 | n.d. | n.d | QVQLVQSG | DIQMTQSP |
| | 46 | 39 | | | | | | AEVKKPGA | SSLSASVG |
| | | | | | | | | SVKVSCKA | DRVTITCR |
| | | | | | | | | SGYTFTSY | ASQGISSY |
| | | | | | | | | GISWVRQA | LAWYQQKP |
| | | | | | | | | PGQGLEW | GKAPKLLI |
| | | | | | | | | MGWISAYN | YDASNLQS |
| | | | | | | | | GNTNYAQK | GVPSRFSG |
| | | | | | | | | LQGRVTMT | SGSGTDFT |
| | | | | | | | | RDTSTSTVY | LTISSLQPE |
| | | | | | | | | MELSSLRS | DFATYYCQ |
| | | | | | | | | EDTAVYYC | QANSFPST |
| | | | | | | | | ARDIGPIDY | FGQGTKVE |
| | | | | | | | | WGQGTLVT | IKR |
| | | | | | | | | VSS | |
| 349 | IGHV3- | IGKV1- | 74 | >50 | >50 | n.d. | n.d | EVQLLESG | DIQMTQSP |
| | 23 | 39 | | | | | | GGLVQPGG | SSLSASVG |
| | | | | | | | | SLRLSCAAS | DRVTITCR |
| | | | | | | | | GFTFSNYG | ASQSISSW |
| | | | | | | | | MHWVRQA | LAWYQQK |
| | | | | | | | | PGKGLEWV | PGKAPKLLI |
| | | | | | | | | SGISSAGSI | YDTSNLET |
| | | | | | | | | TNYADSVK | GVPSRFSG |
| | | | | | | | | GRFTISRDN | SGSGTDFT |
| | | | | | | | | SKNTLYLQ | LTISSLQPE |
| | | | | | | | | MNSLRAED | DFATYYCQ |
| | | | | | | | | TAVYYCAG | QSYTTPWT |
| | | | | | | | | NHAGTTVT | FGQGTRLE |
| | | | | | | | | SEYFQHWG | IKR |
| | | | | | | | | QGTLVTVS | |
| | | | | | | | | S | |
| 178 | IGHV1- | IGKV3- | 72 | 1.7 | >50 | n.d. | n.d | QVQLVQSG | EIVMTQSP |
| | 46 | 15 | | | | | | AEVKKPGA | ATLSVSPG |
| | | | | | | | | SVKVSCKA | ERATLSCK |
| | | | | | | | | SGYTFTDY | ASQSVSGT |
| | | | | | | | | HMHWVRQ | YLAWYQQ |
| | | | | | | | | APGQGLEW | KPGQAPRL |
| | | | | | | | | MGWINPNS | LIYGASTRA |
| | | | | | | | | GGTNYAQK | TGIPARFS |
| | | | | | | | | FQGRVTMT | GSGSGTEF |
| | | | | | | | | RDTSTSTVY | TLTISSLQS |
| | | | | | | | | MELSSLRS | EDFAVYYC |
| | | | | | | | | EDTAVYYC | LQTHSYPP |
| | | | | | | | | ARDISSWY | TFGQGTKV |
| | | | | | | | | EITKFDPWG | EIKR |
| | | | | | | | | QGTLVTVS | |
| | | | | | | | | S | |
| 108 | IGHV1- | IGKV1- | 72 | 0.37 | >50 | n.d. | n.d | QVQLVQSG | DIQMTQSP |
| | 46 | 39 | | | | | | AEVKKPGA | SSLSASVG |
| | | | | | | | | SVKVSCKA | DRVTITCR |
| | | | | | | | | SGYIFSRYA | ASQVITNNL |
| | | | | | | | | IHWVRQAP | AWYQQKP |
| | | | | | | | | GQGLEWM | GKAPKLLIY |
| | | | | | | | | GWMNPISG | DASTLETG |
| | | | | | | | | NTDYAPNF | VPSRFSGS |
| | | | | | | | | QGRVTMTR | GSGTDFTL |
| | | | | | | | | DTSTSTVY | TISSLOPED |
| | | | | | | | | MELSSLRS | FATYYCQQ |
| | | | | | | | | EDTAVYYC | SYTFPYTF |
| | | | | | | | | AKDGSQLA | GQGTKVEI |
| | | | | | | | | YLVEYFQH | KR |
| | | | | | | | | WGQGTLVT | |
| | | | | | | | | VSS | |
| 128 | IGHV1- | IGKV1- | 57 | 3.5 | >50 | n.d. | n.d | QVQLVQSG | DIQMTQSP |
| | 46 | 39 | | | | | | AEVKKPGA | SSLSASVG |
| | | | | | | | | SVKVSCKA | DRVTITCR |
| | | | | | | | | SGYTFTHY | ASQNISRY |
| | | | | | | | | YMHWVRQ | LNWYQQK |
| | | | | | | | | APGQGLEW | PGKAPKLLI |
| | | | | | | | | MGIINPSSS | YDASNLET |
| | | | | | | | | SASYSQKF | GVPSRFSG |
| | | | | | | | | QGRVTMTR | SGSGTDFT |
| | | | | | | | | DTSTSTVY | LTISSLQPE |
| | | | | | | | | MELSSLRS | DFATYYCQ |
| | | | | | | | | EDTAVYYC | QANGFPPT |
| | | | | | | | | ARDGRYGS | FGQGTKLE |
| | | | | | | | | GSYPFDYW | IKR |
| | | | | | | | | GQGTLVTV | |
| | | | | | | | | SS | |
| 160 | IGHV3- | IGKV1- | 7.7 | 0.22 | >50 | n.d. | n.d | QVQLVQSG | DIQMTQSP |
| | 23 | 39 | | | | | | AEVKKPGA | SSLSASVG |
| | | | | | | | | SVKVSCKA | DRVTITCR |
| | | | | | | | | SGYTFTGH | ASQSVSS |
| | | | | | | | | DMHWVRQ | WLAWYQQ |
| | | | | | | | | APGQGLEW | KPGKAPKL |
| | | | | | | | | MGIINPSGG | LIYAASSLQ |
| | | | | | | | | STSYAQKF | SGVPSRFS |
| | | | | | | | | QGRVTMTR | GSGSGTDF |
| | | | | | | | | DTSTSTVY | TLTISSLQP |
| | | | | | | | | MELSSLRS | EDFATYYC |
| | | | | | | | | EDTAVYYC | QQGYTTPY |
| | | | | | | | | ARANSLRY | TFGQGTKL |
| | | | | | | | | YYGMDVW | |
| | | | | | | | | GQGTMVTV | |
| | | | | | | | | SS | |
| 368 | IGHV1- | IGKV2- | nb | 0.073 | >50 | n.d. | n.d | QVQLVQSG | EIKR |
| | 69 | 28 | | | | | | AEVKKPGS | DIVMTQSP |
| | | | | | | | | SVKVSCKA | LSLPVTPG |
| | | | | | | | | SGYTFTSY | EPASISCR |
| | | | | | | | | DINWVRQA | SSQSLLHS |
| | | | | | | | | PGQGLEW | NGYNYLD |
| | | | | | | | | MGAIMPMF | WYLQKPG |
| | | | | | | | | GTANYAQK | QSPQLLIYL |
| | | | | | | | | FQGRVTITA | GSNRASGV |
| | | | | | | | | DESTSTAY | PDRFSGSG |
| | | | | | | | | MELSSLRS | SGTDFTLKI |
| | | | | | | | | EDTAVYYC | SRVEAEDV |
| | | | | | | | | ARGSSGYY | GVYYCMQ |
| | | | | | | | | YGWGQGTL | ALQTPATF |
| | | | | | | | | VTVSS | GPGTKVDI |
| | | | | | | | | | KR |
| 192 | IGHV1- | IGKV2- | nb | 0.79 | >50 | n.d. | n.d | QVQLVQSG | DIVMTQSP |
| | 69 | 28 | | | | | | AEVKKPGS | LSLPVTPG |
| | | | | | | | | SVKVSCKA | EPASISCR |
| | | | | | | | | SGGTFSSY | SSQSLLHS |
| | | | | | | | | AISWVRQA | NGYNYLD |
| | | | | | | | | PGQGLEW | WYLQKPGQ |
| | | | | | | | | MGWINPNS | SPQLLIYA |
| | | | | | | | | GGANYAQK | ASSLQSGV |
| | | | | | | | | FQGRVTITA | PDRFSGSG |
| | | | | | | | | DESTSTAY | SGTDFTLKI |
| | | | | | | | | MELSSLRS | SRVEAEDV |
| | | | | | | | | EDTAVYYC | GVYYCMQ |
| | | | | | | | | STYYYDSS | ALQTPYTF |
| | | | | | | | | GYSTDYWG | GQGTKLEI |
| | | | | | | | | QGTLVTVS | KR |
| | | | | | | | | S | |
| 158 | IGHV1- | IGKV1- | 172 | 0.1 | >50 | n.d. | n.d | QVQLVQSG | DIQMTQSP |
| | 46 | 39 | | | | | | AEVKKPGA | SSLSASVG |
| | | | | | | | | SVKVSCKA | DRVTITCR |
| | | | | | | | | SGYTFTGY | ASQSISRYL |
| | | | | | | | | YMHWVRQ | NWYQQKP |
| | | | | | | | | APGQGLEW | GKAPKLLIY |
| | | | | | | | | MGWINPLN | DASNLESG |
| | | | | | | | | GGTNFAPK | VPSRFSGS |
| | | | | | | | | FQGRVTMT | GSGTDFTL |
| | | | | | | | | RDTSTSTVY | TISSLQPED |
| | | | | | | | | MELSSLRS | FATYYCQQ |
| | | | | | | | | EDTAVYYC | ANSFPLTF |
| | | | | | | | | ARDPGGSY | GGGTKVDI |
| | | | | | | | | SNDAFDIW | KR |
| | | | | | | | | GQGTLVTV | |
| | | | | | | | | SS | |
| 180 | IGHV1- | IGKV2- | nb | 0.89 | >50 | n.d. | n.d | QVQLVQSG | DIVMTQSP |
| | 69 | 28 | | | | | | AEVKKPGS | LSLPVTPG |
| | | | | | | | | SVKVSCKA | EPASISCR |
| | | | | | | | | SGYTFTSY | SSQSLLHS |
| | | | | | | | | AMHWVRQA | NGYNYLD |
| | | | | | | | | PGQGLEW | WYLQKPGQ |
| | | | | | | | | MGRISPRS | SPQLLIYA |
| | | | | | | | | GGTKYAQR | ASSLQSGV |
| | | | | | | | | FQGRVTIT | PDRFSGSG |
| | | | | | | | | ADESTSTA | SGTDFTLK |
| | | | | | | | | YMELSSLR | ISRVEAED |
| | | | | | | | | SEDTAVYY | VGVYYCQQ |
| | | | | | | | | CAREAVAG | YYSSPYTF |
| | | | | | | | | THPQAGDF | GQGTKLEI |
| | | | | | | | | DLWGRGTL | KR |
| | | | | | | | | VTVSS | |
| 254 | IGHV3- | IGKV1- | 127 | 9.3 | >50 | n.d. | n.d | EVQLLESG | DIQMTQSP |
| | 23 | 39 | | | | | | GGLVQPGG | SSLSASVG |
| | | | | | | | | SLRLSCAA | DRVTITCR |
| | | | | | | | | SGFTFSSS | ASQGISSY |
| | | | | | | | | AMHWVRQA | LAWYQQKP |
| | | | | | | | | PGKGLEWV | GKAPKLLI |
| | | | | | | | | SAIGTGGD | YDASSLQI |
| | | | | | | | | TYYADSVK | GVPSRFSG |
| | | | | | | | | RGFTISRD | SGSGTDFT |
| | | | | | | | | NSKNTLYL | LTISSLOPE |
| | | | | | | | | QMNSLRAE | DFATYYCL |
| | | | | | | | | DTAVYYCA | QSYSTPPW |
| | | | | | | | | REGDGYNF | TFGQGTKV |
| | | | | | | | | YFDYWGQG | EIKR |
| | | | | | | | | TLVTVSS | |
| 120 | IGHV1- | IGKV3- | 24 | 7.2 | >50 | n.d. | n.d | QVQLVQSG | EIVMTQSP |
| | 46 | 15 | | | | | | AEVKKPGA | ATLSVSPG |
| | | | | | | | | SVKVSCKA | ERATLSCR |
| | | | | | | | | SGYTFTSY | ASQSVSSR |
| | | | | | | | | DINWVRQA | YLAWYQQK |
| | | | | | | | | PGQGLEW | PGQAPRLL |
| | | | | | | | | MGMIDPSG | IYGASTRA |
| | | | | | | | | GSTSYAQK | TGIPARFS |
| | | | | | | | | FQGRVTMT | GSGSGTEF |
| | | | | | | | | RDTSTSTVY | TLTISSLQ |
| | | | | | | | | MELSSLRS | SEDFAVYY |
| | | | | | | | | EDTAVYYC | CQQYYTTP |
| | | | | | | | | AKDFGGGT | RTFGQGTR |
| | | | | | | | | RYDYWYFD | ELIKR |
| | | | | | | | | LWGRGTLV | |
| | | | | | | | | TVSS | |
| 64 | IGHV3- | IGKV1- | 97 | 14 | >50 | n.d | n.d | EVQLLESG | DIQMTQSP |
| | 23 | 39 | | | | | | GGLVQPGG | SSLSASVG |
| | | | | | | | | SLRLSCAAS | DRVTITCR |
| | | | | | | | | GFPFSQHG | ASQGISSH |
| | | | | | | | | MHWVRQA | LAWYQQKP |
| | | | | | | | | PGKGLEWV | GKAPKLLI |
| | | | | | | | | SAIDRSGSY | YDASNLET |
| | | | | | | | | IYYADSVKG | GVPSRFSG |
| | | | | | | | | RFTISRDNS | SGSGTDFT |
| | | | | | | | | KNTLYLQM | LTISSLQP |
| | | | | | | | | NSLRAEDT | EDFATYYC |
| | | | | | | | | AVYYCARD | QQTYSTPW |
| | | | | | | | | TYGGKVTY | TFGQGTKV |
| | | | | | | | | FDYWGQGT | EIKR |
| | | | | | | | | LVTVSS | |
| 298 | IGHV1- | IGKV4- | 24 | 0.00011 | 2.8 | 0.0057 | 2.2 | QVQLVQSG | DIVMTQSP |
| | 46 | 1 | | | | | | AEVKKPGA | DSLAVSLG |
| | | | | | | | | SVKVSCKA | ERATINCK |
| | | | | | | | | SGGTFSTY | SSQSVLYS |
| | | | | | | | | GISWVRQA | SNNKNYLA |
| | | | | | | | | PGQGLEWM | WYQQKPGQ |
| | | | | | | | | GWISPNS | PPKLLIY |
| | | | | | | | | GGTDLAQK | WASTRESG |
| | | | | | | | | FQGRVTMT | VPDRFSGS |
| | | | | | | | | RDTSTSTVY | GSGTDFTL |
| | | | | | | | | MELSSLRS | TISSLQAE |
| | | | | | | | | EDTAVYYC | DVAVYYCQ |
| | | | | | | | | ASDPRDDIA | QYYSTPPT |
| | | | | | | | | GGYWGQG | FGQGTKLE |
| | | | | | | | | TLVTVSS | IKR |
| 82 | IGHV1- | IGKV1- | 206 | 0.0022 | 1.6 | 0.21 | 19 | QVQLVQSG | DIQMTQSP |
| | 46 | 39 | | | | | | AEVKKPGA | SSLSASVG |
| | | | | | | | | SVKVSCKA | DRVTITCR |
| | | | | | | | | SGGSFSTS | ASQVISNY |
| | | | | | | | | AFYWVRQA | LAWYQQKP |
| | | | | | | | | PGQGLEW | GKAPKLLIY |
| | | | | | | | | MGWINPYT | DASNLETG |
| | | | | | | | | GGTNYAQK | VPSRFSGS |
| | | | | | | | | FQGRVTMT | GSGTDFTL |
| | | | | | | | | RDTSTSTVY | TISSLQPED |
| | | | | | | | | MELSSLRS | FATYYCQQ |
| | | | | | | | | EDTAVYYC | SFSPPPTF |
| | | | | | | | | ARSRALYG | GQGTRLEI |
| | | | | | | | | SGSYFDYW | KR |
| | | | | | | | | GQGTLVTV | |
| | | | | | | | | SS | |
| 46 | IGHV3- | IGKV1- | 83 | 0.0024 | 2.1 | 0.027 | 19 | EVQLLESG | DIQMTQSP |
| | 23 | 39 | | | | | | GGLVQPGR | SSLSASVG |
| | | | | | | | | SLRLSCAAS | DRVTITCR |
| | | | | | | | | GFTFSSYA | ASQSISSW |
| | | | | | | | | MSWVRQAP | LAWYQQKP |
| | | | | | | | | GKGLEWVS | GKAPKLLI |
| | | | | | | | | TIYSGGSTY | YDASNLET |
| | | | | | | | | YADSVKGR | GVPSRFSG |
| | | | | | | | | FTISRDNSK | SGSGTDFT |
| | | | | | | | | NTLYLQMN | LTISSLQP |
| | | | | | | | | SLRAEDTAV | EDFATYYC |
| | | | | | | | | YYCARGDS | QQSYSTPF |
| | | | | | | | | RDAFDIWG | TFGPGTKV |
| | | | | | | | | QGTMVTVS | DIKR |
| | | | | | | | | S | |
| 324 | IGHV1- | IGKV1- | 111 | 0.0009 | 0.78 | 0.024 | 21 | QVQLVQSG | DIQMTQSP |
| | 69 | 39 | | | | | | AEVKKPGA | SSLSASVG |
| | | | | | | | | SVKVSCKA | DRVTITCR |
| | | | | | | | | SGGTFNNY | ASQSITTYL |
| | | | | | | | | GISWVRQA | NWYQQKPG |
| | | | | | | | | PGQGLEW | KAPKLLIY |
| | | | | | | | | MGWMNPN | DASNLETG |
| | | | | | | | | SGNTGYAQ | VPSRFSGS |
| | | | | | | | | KFQGRVTM | GSGTDFTL |
| | | | | | | | | TRDTSTSTV | TISSLQPED |
| | | | | | | | | YMELSSLR | FATYYCQQ |
| | | | | | | | | SEDTAVYY | SYSTPPTF |
| | | | | | | | | CARVGDYG | GQGTKVEI |
| | | | | | | | | DYIVSPFDL | KR |
| | | | | | | | | WGRGTLVT | |
| | | | | | | | | VSS | |
| 236 | IGHV1- | IGKV2- | 145 | 0.00047 | 0.057 | 0.028 | 5.5 | QVQLVQSG | DIVMTQSP |
| | 69 | 28 | | | | | | AEVKKPGA | LSLPVTPG |
| | | | | | | | | SVKVSCKA | EPASISCR |
| | | | | | | | | SGGTFTSY | SSQSLLHS |
| | | | | | | | | GINWVRQA | NGYNYLD |
| | | | | | | | | PGQGLEW | WYLQKPG |
| | | | | | | | | MGWMNPN | QSPQLLIYL |
| | | | | | | | | SGNTGYAQ | GSNRASGV |
| | | | | | | | | KFQGRVTM | PDRFSGSG |
| | | | | | | | | TRDTSTSTV | SGTDFTLKI |
| | | | | | | | | YMELSSLR | SRVEAEDV |
| | | | | | | | | SEDTAVYY | GVYYCMQ |
| | | | | | | | | CASRGIQLL | ALQTPPTF |
| | | | | | | | | PRGMDVW | GQGTRLEI |
| | | | | | | | | GQGTTVTV | KR |
| | | | | | | | | SS | |
| 52 | IGHV1- | IGKV1- | 12 | 0.0002 | 0.55 | 0.27 | 6.2 | QVQLVQSG | DIQMTQSP |
| | 69 | 39 | | | | | | AEVKKPGS | SSLSASVG |
| | | | | | | | | SVKVSCKA | DRVTITCR |
| | | | | | | | | SGYTFTSY | ASQGISNN |
| | | | | | | | | GISWVRQA | LNWYQQK |
| | | | | | | | | PGQGLEW | PGKAPKLLI |
| | | | | | | | | MGGIIPMFG | YAASSLES |
| | | | | | | | | TTNYAQKF | GVPSRFSG |
| | | | | | | | | QGRVTITAD | SGSGTDFT |
| | | | | | | | | KSTSTAYM | LTISSLQPE |
| | | | | | | | | ELSSLRSED | DFATYYCQ |
| | | | | | | | | TAVYYCAR | QGNGFPLT |
| | | | | | | | | DRGDTIDY | FGPGTKVD |
| | | | | | | | | WGQGTLVT | IKR |
| | | | | | | | | VSS | |
| 80 | IGHV1- | IGKV4- | 142 | 0.0013 | 0.1 | 0.32 | 12.7 | QVQLVQSG | DIVMTQSP |
| | 69 | 1 | | | | | | AEVKKPGS | DSLAVSLG |
| | | | | | | | | SVKVSCKA | ERATINCK |
| | | | | | | | | SGGTFNRY | SSQSVLYS |
| | | | | | | | | AFSWVRQA | SNNKNYLA |
| | | | | | | | | PGQGLEWM | WYQQKPG |
| | | | | | | | | GGIIPIFGT | QPPKLLIY |
| | | | | | | | | ANYAQKFQ | WASTRES |
| | | | | | | | | GRVTITADE | GVPDRFSG |
| | | | | | | | | STSTAYMEL | SGSGTDFT |
| | | | | | | | | SSLRSEDTA | LTISSLQAE |
| | | | | | | | | VYYCARST | DVAVYYCQ |
| | | | | | | | | RELPEVVD | QYYSAPLT |
| | | | | | | | | WYFDLWGR | FGGGTKVE |
| | | | | | | | | GTLVTVSS | IKR |

### MATERIALS AND METHODS REFERENCES

46. Kabsch, W. et al. XDS. Acta Crystallogr. Sect. D Biol. Crystallogr. 66, 125-132 (2010).
47. McCoy, A. J. et al. Phaser crystallographic software. J. Appl. Crystallogr. 40, 658-674 (2007).
48. Adams, P. D. et al. PHENIX: A comprehensive Python-based system for macromolecular structure solution. Acta Crystallogr. Sect. D Biol. Crystallogr. 66, 213-221 (2010).
49. Emsley, P., Lohkamp, B., Scott, W. G. & Cowtan, K. Features and development of Coot. Acta Crystallogr. Sect. D Biol. Crystallogr. 66, 486-501 (2010).
50. Morin, A. et al. Collaboration gets the most out of software. Elife 2, (2013).
51. Marr, C. R., Benlekbir, S. & Rubinstein, J. L. Fabrication of carbon films with ˜500 nm holes for cryo-EM with a direct detector device. J. Struct. Biol. 185, 42-47 (2014).
52. Punjani, A., Rubinstein, J. L., Fleet, D. J. & Brubaker, M. A. CryoSPARC: Algorithms for rapid unsupervised cryo-EM structure determination. Nat. Methods 14, 290-296 (2017).
53. Zi Tan, Y. et al. Addressing preferred specimen orientation in single-particle cryo-EMthrough tilting. Nat. Methods 14, (2017).
54. Zivanov, J. et al. New tools for automated high-resolution cryo-EM structure determination in RELION-3. Elife 9, e42166 (2018).
55. Scheres, S. H. W. RELION: Implementation of a Bayesian approach to cryo-EM structure determination. J. Struct. Biol. 180, 519-530 (2012).
56. Punjani, A. & Fleet, D. 3D Variability Analysis: Directly resolving continuous flexibility and discrete heterogeneity from single particle cryo-EM images. bioRxiv (2020).
57. Punjani, A., Zhang, H. & Fleet, D. Non-uniform refinement: Adaptive regularization improves single particle cryo-EM reconstruction. bioRxiv (2019).
58. Grant, T., Rohou, A. & Grigorieff, N. CisTEM, user-friendly software for single-particle image processing. Elife 7, e35383 (2018).
59. Asarnow, D., Palovcak, E. & Cheng, Y. asarnow/pyem: UCSF pyem v0. 5. (2019).

### Results

### Avidity Enhances Neutralization Potency

We used the self-assembly of the light chain of human apoferritin to multimerize antigen binding moieties targeting the SARS-CoV-2 S glycoprotein. Apoferritin protomers self-assemble into an octahedrally symmetric structure with an ˜6 nm hydrodynamic radius (Rh) composed of 24 identical polypeptides³³. The N terminus of each apoferritin subunit points outwards of the spherical nanocage and is therefore accessible for the genetic fusion of proteins of interest. Upon folding, apoferritin protomers act as building blocks that drive the multimerization of the 24 proteins fused to their N termini (FIG. 1a).

First, we investigated the impact of multivalency on the ability of the single chain variable domain VHH-72 to block viral infection. VHH-72 has been previously described to neutralize SARS-CoV-2 when fused to a Fc domain, but not in its monovalent format³¹. The light chain of human apoferritin displaying 24 copies of VHH-72 assembled into monodisperse, well-formed spherical particles (FIG. 1b, c) and showed an enhanced binding avidity to the S glycoprotein (FIG. 1d) in comparison to bivalent VHH-72-Fc. Strikingly, display of VHH-72 on the light chain of human apoferritin achieved a ˜10,000-fold increase in neutralization potency against SARS-CoV-2 pseudovirus (PsV) compared to the conventional Fc fusion (FIG. 1e), demonstrating the power of avidity to transform binding moieties into potent neutralizers.

Multabodies have IgG-Like Properties

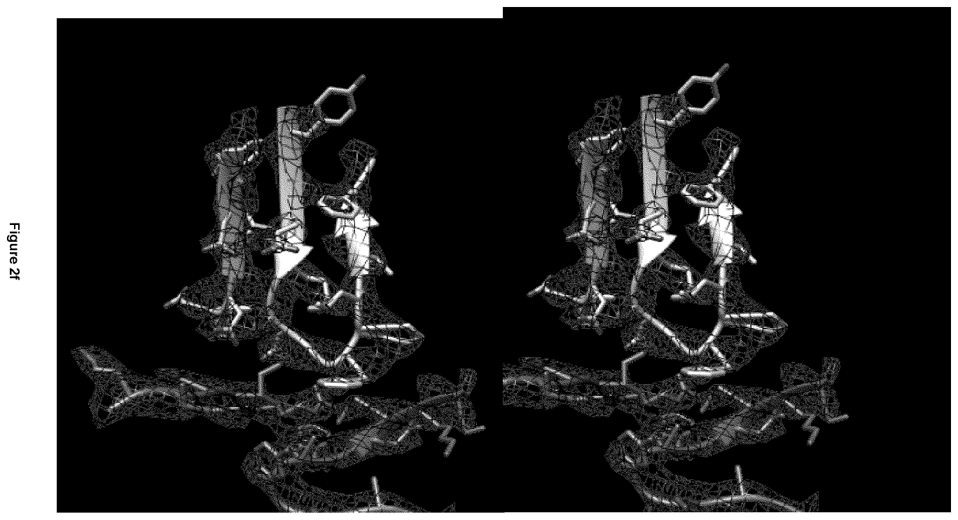

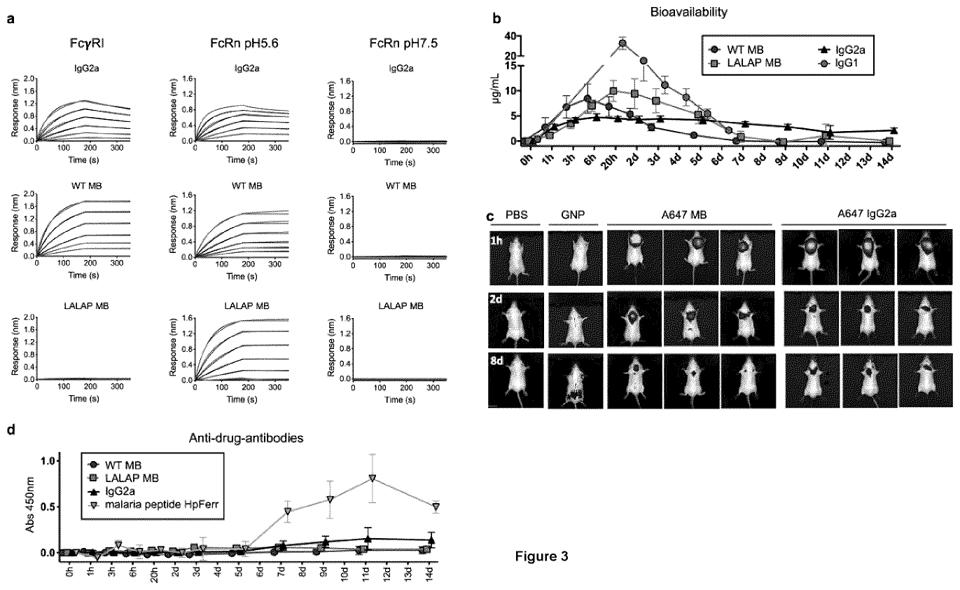

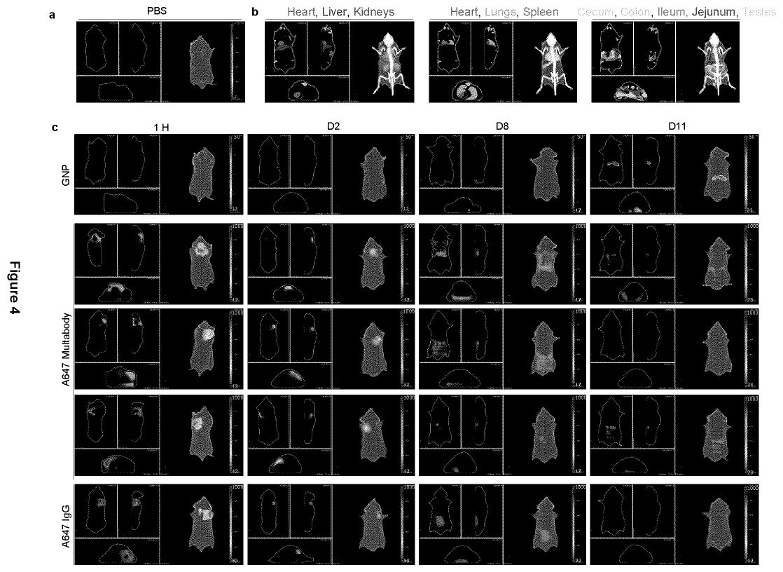

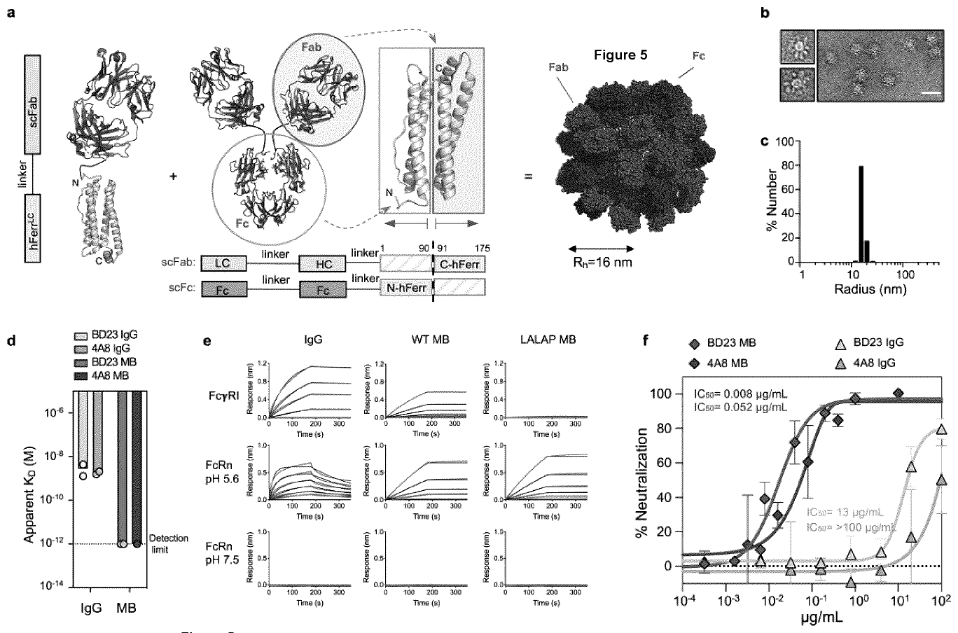

The Fc confers IgGs in vivo half-life and effector functions through interaction with neonatal Fc receptor (FcRn) and Fc gamma receptors (FcγR), respectively. To confer these IgG-like properties to our multimeric scaffold, we next sought to incorporate both binding moieties and Fc domains. Because a Fab is a hetero-dimer consisting of a light and a heavy chain, and the Fc is a homodimer, we created single-chain Fab (scFab) and single-chain Fc (scFc) polypeptide constructs. scFab and scFc domains were directly fused to the N terminus of the apoferritin protomer. For in vivo proof-of-principle experiments, we generated a species-matched surrogate molecule that consists of mouse light chain apoferritin fusions to a mouse scFab and a mouse scFc (IgG2a subtype). Binding kinetics showed that the resulting MB molecule binds mouse FcRn in a pH dependent manner-binding at endosomal pH (5.6) and no binding at physiological pH (7.4)—similar to the parental IgG (FIG. 3a). Expectedly, binding to the high-affinity mouse FcγR1 was enhanced through avidity effects in comparison to the parental IgG. Hence, we generated a modified mouse scFc version that includes the FcγR-silencing mutations LALAP to lower Fc binding in a multimeric context (FIG. 3a). Subcutaneous administration of MBs in C57BL/6 or BALB/c mice was well tolerated with no decrease in body weight or visible adverse events. The MB showed favorable IgG-like serum half-life (FIG. 3b), with a prolonged detectable titer in the sera for the lower FcγR-binding MB (LALAP Fc sequence) compared to the WT MB, indicative of a role for the Fc in dictating in vivo bioavailability. Live 2D and 3D-imaging revealed that the fluorescently-labeled MB biodistributed systemically like the corresponding IgG, without accumulation in any specific tissue (FIG. 3c and FIG. 4). In contrast, 15 nm gold nanoparticles (GNP), which have a similar Rh as MBs, rapidly disseminated from the site of injection (FIG. 3c and FIG. 4). Presumably because all sequences derived from the host, the surrogate mouse MB did not induce an anti-drug antibody response in mice (FIG. 3d), thus further highlighting the IgG-like properties of the MB platform.

### Protein Engineering to Achieve Higher Valency

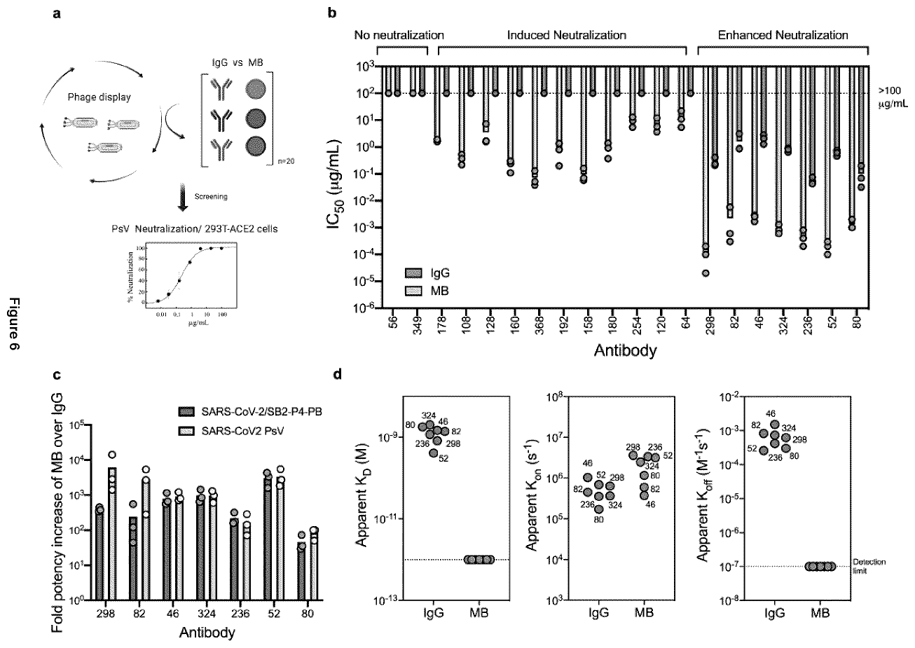

In view of these favorable results for a mouse MB surrogate, we aimed to generate fully-human MBs derived from the previously reported IgG BD23¹² and IgG 4A8¹³ that target the SARS-CoV-2 spike RBD and N-terminal domain (NTD), respectively. Addition of scFcs into the MB reduces the number of scFabs that can be multimerized. In order to endow the MB platform with Fc without compromising Fab avidity and hence neutralization potency, we engineered the apoferritin protomer to accommodate more than 24 components per particle. Based on its four-helical bundle fold, the human apoferritin protomer was split into two halves: the two N-terminal a helices (N-Ferritin) and the two C-terminal a helices (C-Ferritin). In this configuration, the scFc fragment of human IgG1 and the scFab of anti-SARS-CoV-2 IgGs were genetically fused at the N terminus of each apoferritin half, respectively. Split apoferritin complementation led to hetero-dimerization of the two halves and consequently resulted in a very efficient hetero-dimerization process of the fused proteins. Co-expression of the scFab-C-Ferritin and scFc-N-Ferritin genes together with the scFab-Ferritin gene in excess resulted in a full apoferritin self-assembly that displays high numbers of scFab and low numbers of scFc on the nanocage periphery (FIG. 5a and Materials and Methods). Conveniently, this design allows for the straightforward purification of the MB using Protein A akin to IgG purification.

This split MB design forms 16 nm Rh spherical particles with an uninterrupted ring of density and regularly spaced protruding scFabs and scFc (FIG. 5b, c). Hence, the MB is on the lower size range of natural IgMs³⁴, but packs more weight on a similar size to achieve high multi-valency. Binding kinetics experiments demonstrated that high binding avidity of the MB for the Spike was preserved upon addition of Fc fragments (FIG. 5d and Table 5). Binding to human FcγRI and FcRn at both pH 5.6 and 7.4 confirmed that scFc was properly folded in the split MB design (Tables 6 and 7). In addition, the LALAP mutations in the scFc lowered the binding affinity to human FcγRI (FIG. 5e), as previously observed with the surrogate mouse MB (FIG. 3a). SARS-CoV-2 PsV neutralization assays with the split design MBs showed that enhanced binding affinity for the Spike translates into an improved neutralization potency in comparison to their IgG counterparts, with a ˜1600-fold and >2000-fold increase for BD23 and 4A8, respectively (FIG. 5f). Combined, this data supported the further exploration of the MB as an IgG-like platform that confers exquisite binding avidity and PsV neutralization across epitopes on different Spike domains.

TABLE 5
Kinetic constants and affinities to SARS-CoV-2
Spike antigen of Multabodies determined by BLI.

SARS-CoV-2 Spike

| Multabody | kₒₙ [M⁻¹ × s⁻¹] | K_{off} [s⁻¹] | K_{D} [M] |
|---|---|---|---|
| 4A8 MB | 1.08E+05 | <1.0E−07 | <1.0E−12 |
| 4A8 IgG | 1.33E+05 | 1.91E−04 | 1.42E−09 |
| BD23 MB | 9.57E+05 | <1.0E−07 | <1.0E−12 |
| BD23 IgG | 2.17E+05 | 9.37E−04 | 4.33E−09 |

TABLE 6
Kinetic constants and affinities to human FcRn of human Ferritin Multabodies
derived from BD23 Antibody (IgG1) targeting SARS-CoV-2.

| | FcRn, pH 5.6 | | | FcRn, pH 7.5 | | |
|---|---|---|---|---|---|---|
| Multabody | kₒₙ [M⁻¹ × s⁻¹] | k_{off} [s⁻¹] | K_{D} [M] | kₒₙ [M⁻¹ × s⁻¹] | k_{off} [s⁻¹] | K_{D} [M] |
| IgG1 control | 5.03E+05 | 3.86E−03 | 7.67E−09 | — | — | No |
| | | | | | | binding |
| WT MB | 2.18E+05 | <1.0E−07 | <1.0E−12 | — | — | No |
| | | | | | | binding |
| LALAP MB | 2.69E+05 | <1.0E−07 | <1.0E−12 | — | — | No |
| | | | | | | binding |
| I253A MB | 2.00E+05 | 4.28E−04 | 2.15E−09 | — | — | No |
| | | | | | | binding |
| LALAP + I253A MB | 4.34E+05 | 6.10E−04 | 1.41E−09 | — | — | No |
| | | | | | | binding |

TABLE 7
Kinetic constants and affinities to human FcyRI of human Ferritin
Multabodiesderived from BD23 Antibody
(IgG1) targeting SARS-CoV-2

| | FcyRI | | |
|---|---|---|---|
| Multabody | kₒₙ [M⁻¹ × s⁻¹] | k_{off} [s⁻¹] | K_{D} [M] |
| IgG1 control | 1.27E+05 | 1.34E−04 | 1.06E−09 |
| WT MB | 7.21E+05 | <1.0E−07 | <1.0E−12 |
| LALAP MB | — | — | No binding |
| I253A MB | 5.01E+05 | <1.0E−07 | <1.0E−12 |
| LALAP I253A | — | — | No binding |
| MB | | | |

Fc mutations of IgG1 backbone evaluated in Multabodies include: LALAP (L234A, L235A and P329G) and I235A, and combinations thereof that decrease antibody binding to FcγR. (Numberings are according to the EU numbering scheme.)

The values determined for kₒₙ, k_{off}, and the resulting equilibrium dissociation constant (K_{D}) for the Multabodies are summarized in Tables 6-10. Binding kinetics showed that the resulting mouse MB molecule binds mouse FcRn in a pH dependent manner—binding at endosomal pH (5.6) and no binding at physiological pH (7.4)—similar to the parental IgG (FIG. 3A). Binding to the high-affinity mouse FcγR1 was enhanced through avidity effects in comparison to the parental IgG. Binding of human MB to human FcγRI and FcRn at endosomal pH confirmed that scFc was properly folded in the split MB design and that LALAP and I253A mutations lowered binding affinities to FcγRI and FcRn, respectively.

TABLE 8
Kinetic constants and affinities to mouse FcRn of mouse Ferritin Multabodies
derived from HD37 Antibody (IgG2a) targeting CD19 determined by BLI.

| | mFcRn, pH 5.6 | | | mFcRn, pH 7.4 | | |
|---|---|---|---|---|---|---|
| Multabody | kₒₙ [M⁻¹ × s⁻¹ ] | k_{off} [s⁻¹ ] | K_{D} [M] | kₒₙ [M⁻¹ × s⁻¹] | k_{off} [s⁻¹] | K_{D} [M] |
| mIgG2a control | 1.16E+05 | 1.23E−04 | 1.06E−09 | — | — | No |
| | | | | | | binding |
| mWT MB | 6.82E+05 | <1.0E−07 | <1.0E−12 | — | — | No |
| | | | | | | binding |
| mLALAP MB | 2.40E+06 | <1.0E−07 | <1.0E−12 | — | — | No |
| | | | | | | binding |

TABLE 9
Kinetic constants and affinities to mouse FcyRI of mouse
Ferritin Multabodies derived from HD37 Antibody (IgG2)
targeting CD19 determined by BLI.

| | mFcyRI | | |
|---|---|---|---|
| Multabody | kₒₙ [M⁻¹ × s⁻¹] | k_{off} [s⁻¹] | K_{D} [M] |
| mIgG2a control | 5.22E+04 | 4.51E−04 | 8.63E−09 |
| mWT MB | 9.39E+05 | <1.0E−07 | <1.0E−12 |
| mLALAP MB | — | — | No binding |

TABLE 10
Kinetic constants and affinities to human FcRn of human Ferritin Multabodies
derived from BD23 Antibody (IgG1) targeting SARS-CoV-2.

| | mFcRn, pH 5.6 | | | mFcRn, pH 7.5 | | |
|---|---|---|---|---|---|---|
| Multabody | kₒₙ [M⁻¹ × s⁻¹ ] | k_{off} [s⁻¹ ] | K_{D} [M] | kₒₙ [M⁻¹ × s⁻¹] | k_{off} [s⁻¹] | K_{D} [M] |
| IgG1 control | 5.03E+05 | 3.86E−03 | 7.67E−09 | — | — | No |
| | | | | | | binding |
| WT MB | 2.18E+05 | <1.0E−07 | <1.0E−12 | — | — | No |
| | | | | | | binding |
| LALAP MB | 2.69E+05 | <1.0E−07 | <1.0E−12 | — | — | No |
| | | | | | | binding |
| 1253A MB | 2.00E+05 | 4.28E−04 | 2.15E−09 | — | — | No |
| | | | | | | binding |
| LALAP +1253A MB | 4.34E+05 | 6.10E−04 | 1.41E−09 | — | — | No |
| | | | | | | binding |

### From Antibody Discovery to Ultrapotent Neutralizers

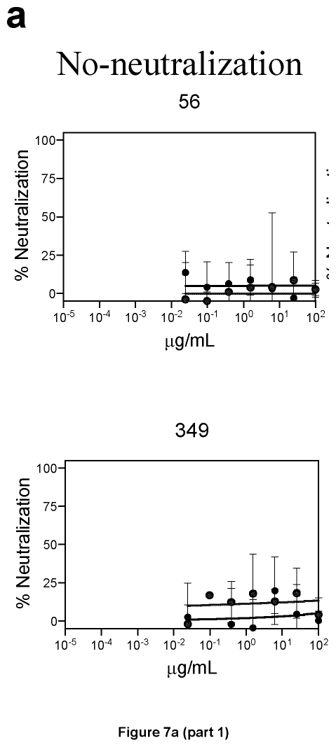

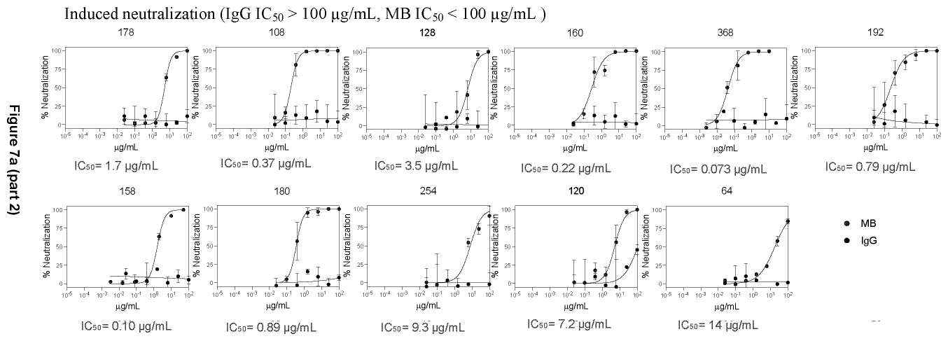

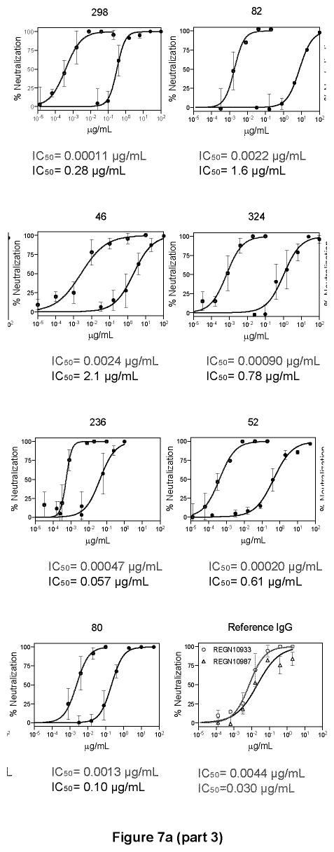

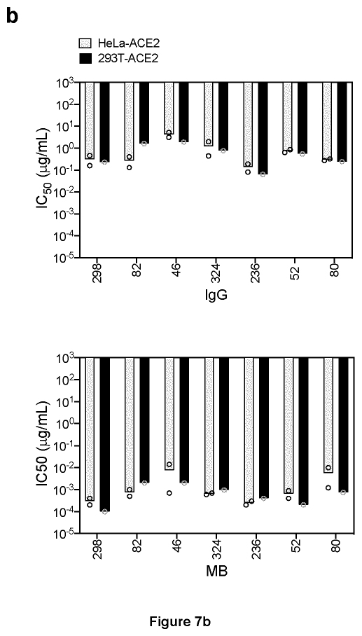

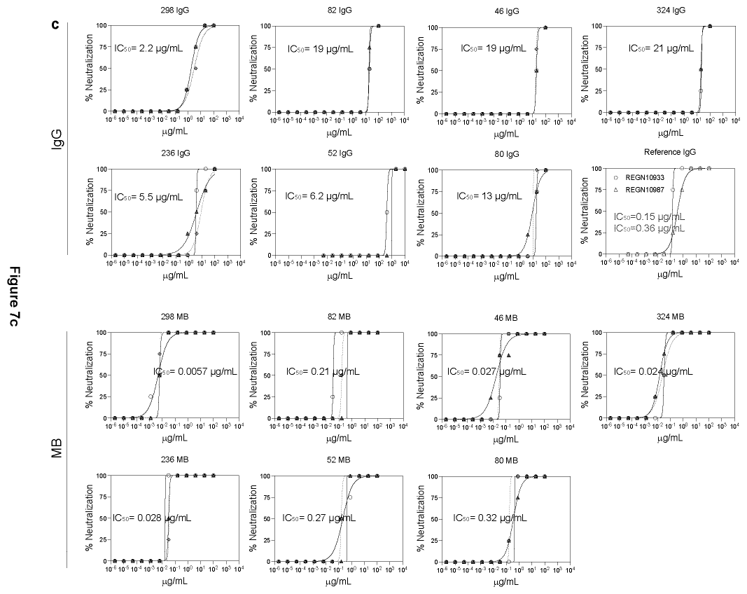

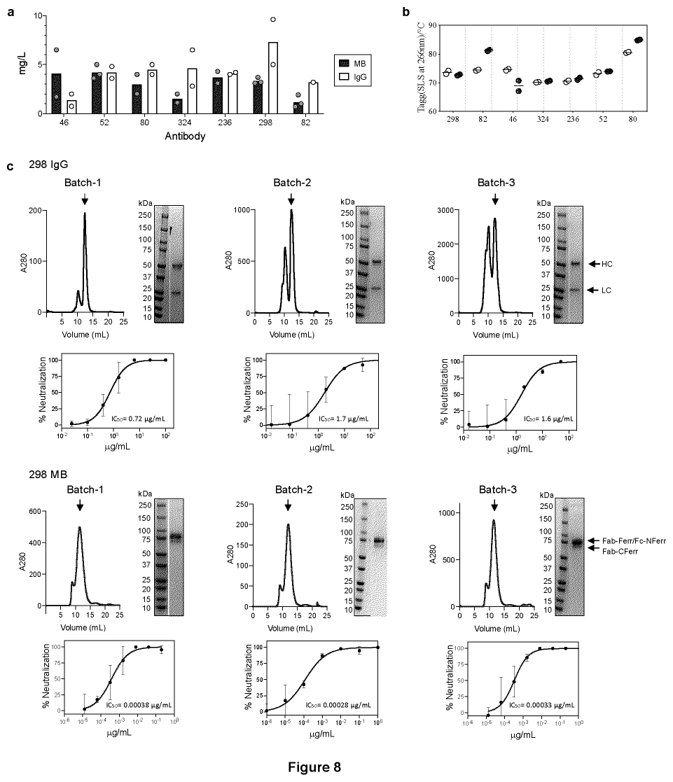

We next assessed the ability of the MB platform to transform mAb binders identified from initial phage display screens into potent neutralizers against SARS-CoV-2 (FIG. 6a). Following standard biopanning protocols against the RBD of SARS-CoV-2, 20 human mAb binders with moderate affinities that range from 10⁻⁶ to 10⁻⁸ M were selected (Table 4; Table 11). These mAbs were produced as full-length IgGs and MBs and their capacity to block viral infection was compared in a neutralization assay against SARS-CoV-2 PsV (FIG. 6b and FIG. 7a). Notably, MB expression yields, homogeneity and thermostability was similar to those of the parental IgG (FIG. 8 and Table 12) and the MB enhanced the potency of 18 out of 20 (90%) IgGs by up to four orders of magnitude (Table 13). The largest increment was observed for mAb 298 which went from a mean VK₅₀ of ˜0.3 μg/mL as an IgG to 0.0001 μg/mL as a MB. Strikingly, 11 mAbs were converted from non-neutralizing IgGs to neutralizing MBs in the tested concentration ranges. Seven MBs displayed exceptional potency with IC₅₀ values between 0.2-2 ng/mL against SARS-CoV-2 PsV using two different target cells (293T-ACE2 and HeLa-ACE2 cells; FIG. 6b and FIG. 7b). PsV neutralization assays using recombinant mAbs REGN10933 and REGN10987 as benchmark showed similar IC₅₀ values (0.0044 and 0.030 μg/mL, respectively) to those previously reported⁸, and thus confirmed the extraordinary potency of the MBs observed in our assays. The enhanced neutralization potency of the MB was further confirmed with authentic SARS-CoV-2 virus for the mAbs with the highest potency (FIG. 6c and FIG. 7c), as also benchmarked with the two recombinant REGN mAbs. The less sensitive neutralization phenotype we observed against authentic virus in comparison to PsV is also in agreement with previous reports^{5,8,9,12}.

TABLE 11
human mAb binders of SARS-CoV-2 RBD.

| | | | RBD |
|---|---|---|---|
| mAb ID | VH | VK | KD (nM) |
| 56 | IGHV1-46 | IGKV1-39 | 23 |
| 349 | IGHV3-23 | IGKV1-39 | 74 |
| 178 | IGHV1-46 | IGKV3-15 | 72 |
| 108 | IGHV1-46 | IGKV1-39 | 72 |
| 128 | IGHV1-46 | IGKV1-39 | 57 |
| 160 | IGHV3-23 | IGKV1-39 | 7.7 |
| 368 | IGHV1-69 | IGKV2-28 | nb |
| 192 | IGHV1-69 | IGKV2-28 | nb |
| 158 | IGHV1-46 | IGKV1-39 | 172 |
| 180 | IGHV1-69 | IGKV2-28 | nb |
| 254 | IGHV3-23 | IGKV1-39 | 127 |
| 120 | IGHV1-46 | IGKV3-15 | 24 |
| 64 | IGHV3-23 | IGKV1-39 | 97 |
| 298 | IGHV1-46 | IGKV4-1 | 24 |
| 82 | IGHV1-46 | IGKV1-39 | 206 |
| 46 | IGHV3-23 | IGKV1-39 | 83 |
| 324 | IGHV1-69 | IGKV1-39 | 111 |
| 236 | IGHV1-69 | IGKV2-28 | 145 |
| 52 | IGHV1-69 | IGKV1-39 | 12 |
| 80 | IGHV1-69 | IGKV4-1 | 142 |

nb = binding below the limit of detection

TABLE 12
Aggregation temperature (T_{agg}) of
Multabodies and related Antibodies.

| Multabody/Antibody | T_{agg} [° C.] |
|---|---|
| MB 298 | 73 |
| MB 82 | 81 |
| MB 46 | 69 |
| MB 324 | 71 |
| MB 236 | 71 |
| MB 52 | 74 |
| MB 80 | 85 |
| IgG 298 | 74 |
| IgG 82 | 75 |
| IgG 46 | 75 |
| IgG 324 | 70 |
| IgG 236 | 70 |
| IgG 52 | 73 |
| IgG 80 | 81 |

TABLE 13
SARS-CoV-2 Neutralization by
RBD-targeting Multabodies

| Multabody | Median IC₅₀ | |
|---|---|---|
| MB 178 | 1.7 | μg/mL (0.74 nM) |
| IgG 178 | >50 | μg/mL (>333 nM) |
| MB 108 | 0.37 | μg/mL (161 pM) |
| IgG 108 | >50 | μg/mL (>333 nM) |
| MB 128 | 3.5 | μg/mL (1.5 nM) |
| IgG 128 | >50 | μg/mL (>333 nM) |
| MB 160 | 0.22 | μg/mL (96 pM) |
| IgG 160 | >50 | μg/mL (>333 nM) |
| MB 368 | 0.073 | μg/mL (32 pM) |
| IgG 368 | >50 | μg/mL (>0333 nM) |
| MB 192 | 0.79 | μg/mL (343 PM) |
| IgG 192 | >50 | μg/mL (>333 nM) |
| MB 158 | 0.10 | μg/mL (43 pM) |
| IgG 158 | >50 | μg/mL (>333 nM) |
| MB 180 | 0.89 | μg/mL (387 pM) |
| IgG 180 | >50 | μg/mL (>333 nM) |
| MB 254 | 9.3 | μg/mL (4.0 nM) |
| IgG 254 | >50 | μg/mL (>333 nM) |
| MB 120 | 7.2 | μg/mL (3.1 nM) |
| IgG 120 | >50 | μg/mL (>333 nM) |
| MB 64 | 14 | μg/mL (6.1 nM) |
| IgG 64 | >50 | μg/mL (>333 nM) |
| MB 56 | >50 | μg/mL (>0.33 μM) |
| IgG 56 | >50 | μg/mL (>333 nM) |
| MB 349 | >50 | μg/mL (>0.33 uM) |
| IgG 349 | >50 | μg/mL (>333 nM) |
| MB 298 | 0.00011 | μg/mL (0.048 pM) |
| IgG 298 | 0.28 | μg/mL (1.9 nM) |
| MB 82 | 0.0022 | μg/mL (0.95 pM) |
| IgG 82 | 1.6 | μg/mL (11 nM) |
| MB 46 | 0.0024 | μg/mL (1.0 pM) |
| IgG 46 | 2.1 | μg/mL (14 nM) |
| MB 324 | 0.0009 | μg/mL (0.39 pM) |
| IgG 324 | 0.78 | μg/mL (5.2 nM) |
| MB 236 | 0.00047 | μg/mL (0.20 pM) |
| IgG 236 | 0.057 | μg/mL (0.38 nM) |
| MB 52 | 0.0002 | μg/mL (0.087 pM) |
| IgG 52 | 0.61 | μg/mL (4.1 nM) |
| MB 80 | 0.0013 | μg/mL (0.56 pM) |
| IgG 80 | 0.1 | μg/mL (0.67 nM) |
| Reference IgG | 0.0044 | μg/mL (29 pM) |
| (REGN 10933) | | |
| Reference IgG | 0.030 | μg/mL (0.20 nM) |
| (REGN 10987) | | |

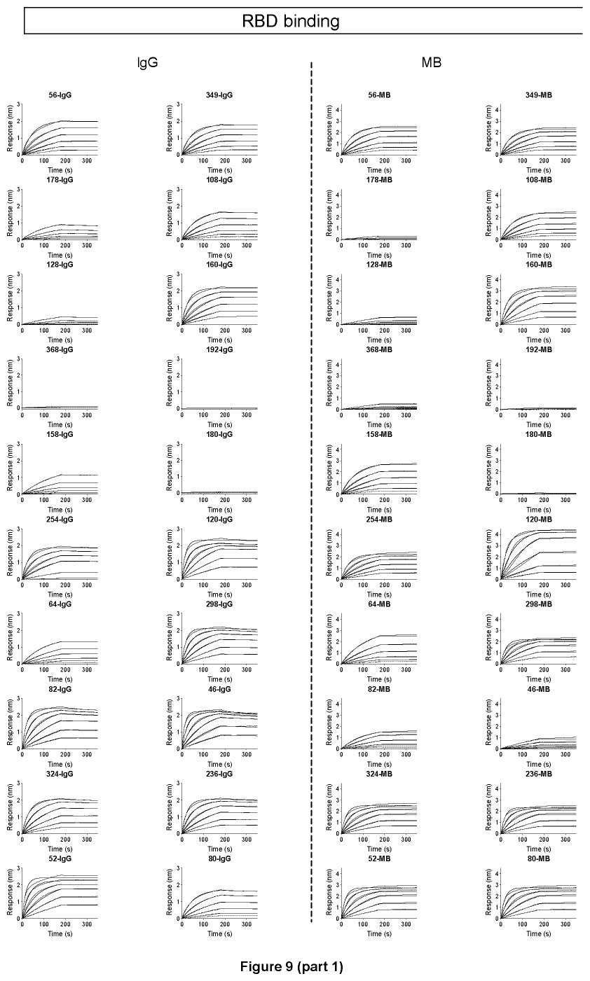

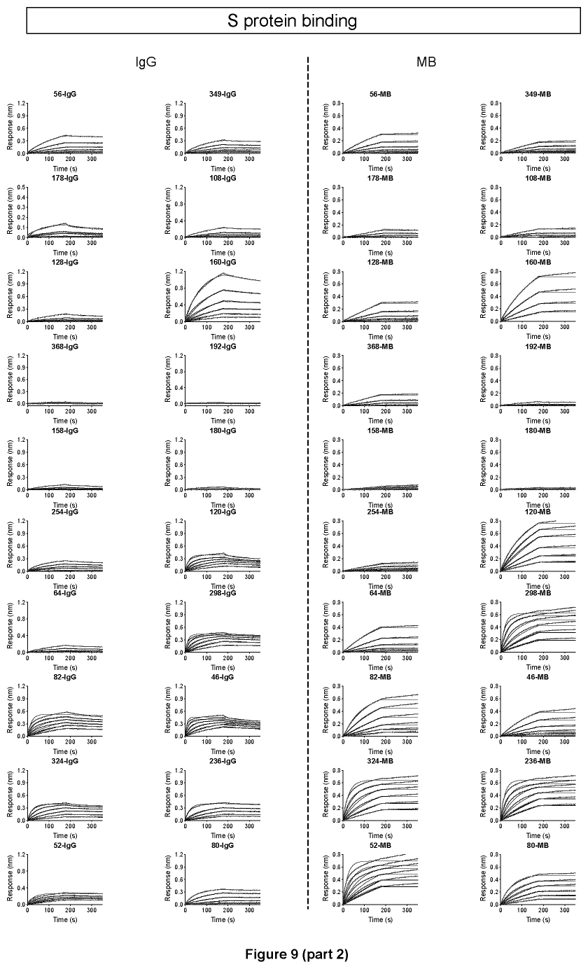

Retrospectively, all IgGs and MBs were tested for their ability to bind to the Spike glycoprotein and the RBD of SARS-CoV-2 (FIG. 9). Increased avidity resulted in higher apparent binding affinities with no detectable off-rates against the Spike glycoprotein, most likely due to inter-spike crosslinking that translates into high neutralization potency (FIG. 6b-d and FIG. 9). Overall, the data show that the MB platform is compatible with rapid delivery of ultrapotent IgG-like molecules even when starting with mAbs of modest neutralization characteristics.

### Epitope Mapping

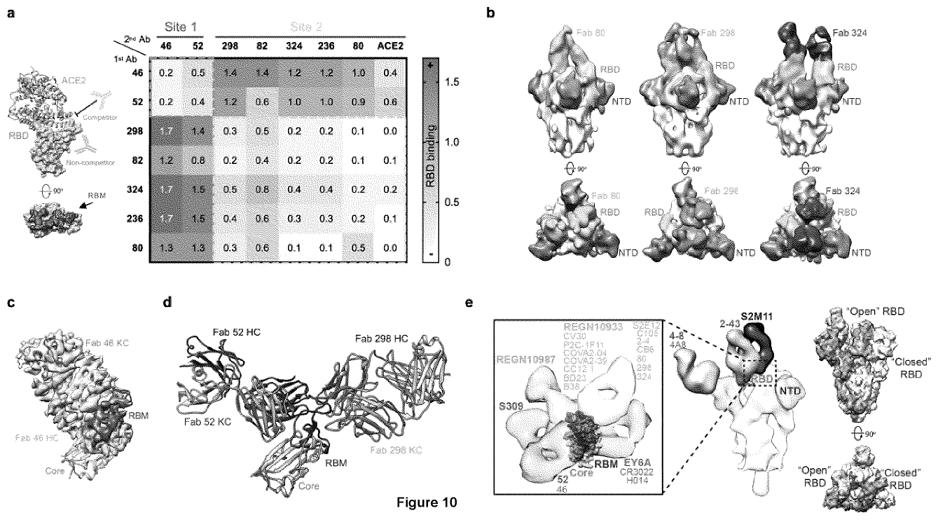

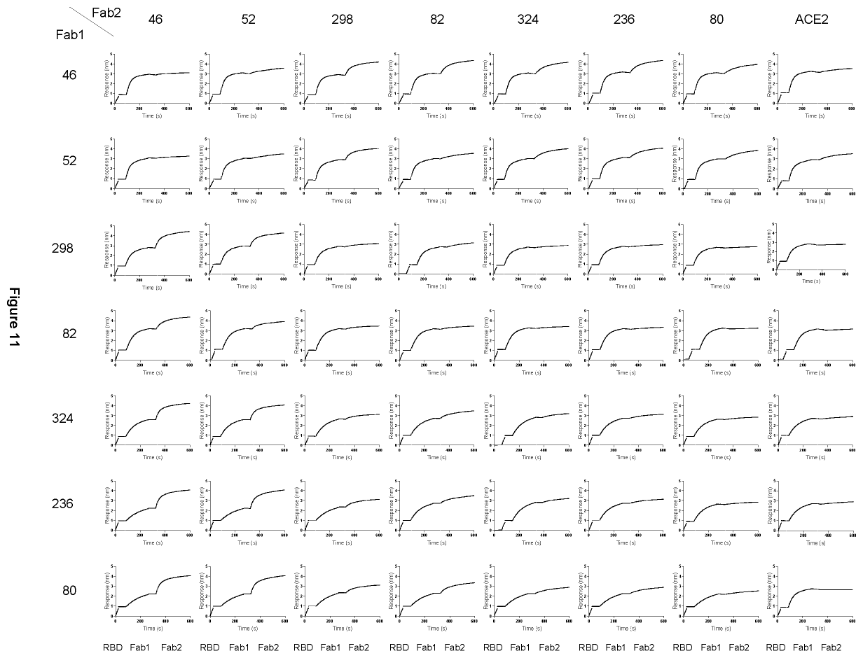

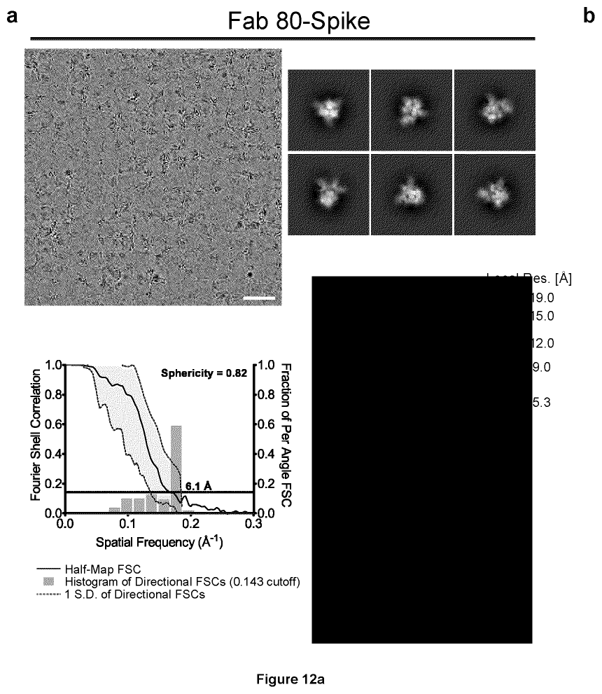

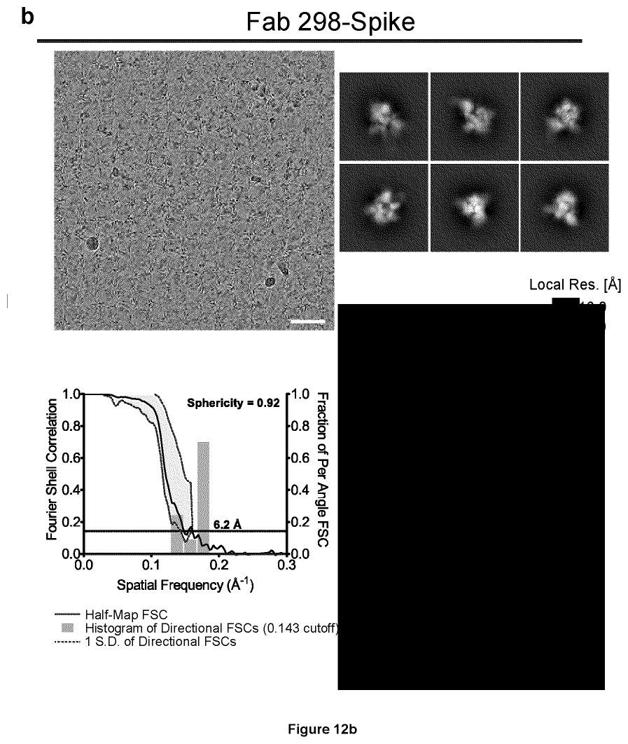

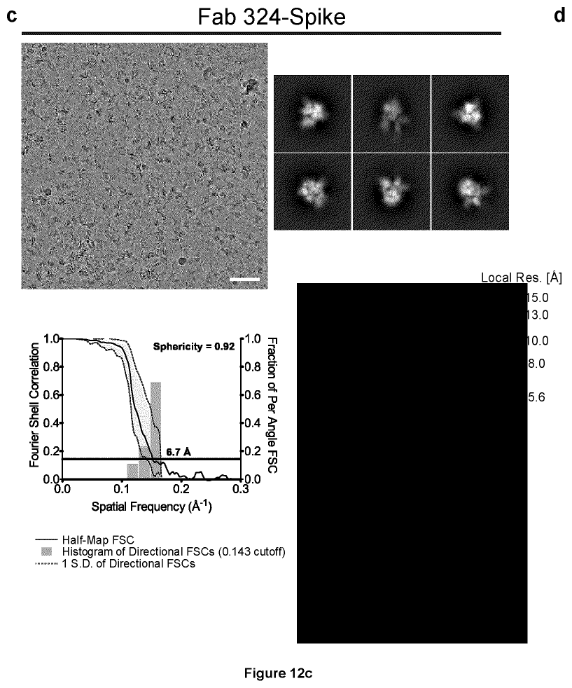

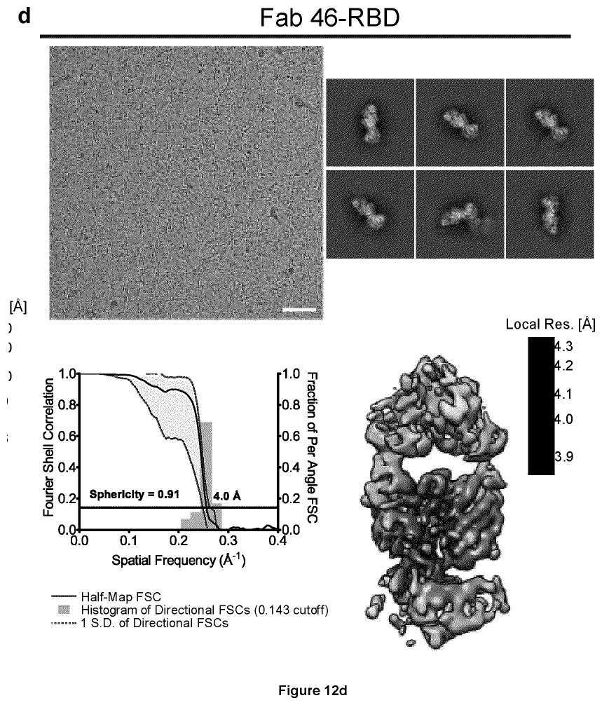

Based on their neutralization potency, seven mAbs were selected for further characterization: 298 (IGHV1-46/IGKV4-1), 82 (IGHV1-46/IGKV1-39), 46 (IGHV3-23/IGKV1-39), 324 (IGHV1-69/IGKV1-39), 236 (IGHV1-69/IGKV2-28), 52 (IGHV1-69/IGKV1-39), and 80 (IGHV1-69/IGKV4-1) (FIG. 6b and Table 4). Epitope binning experiments showed that these mAbs target two main sites on the RBD, with one of these bins overlapping with the ACE2 binding site (FIG. 10a and FIG. 11). Cryo-EM structures of Fab-SARS-CoV-2 S complexes at a global resolution of ˜6-7 Å confirmed that mAbs 324, 298, and 80 bind overlapping epitopes (FIG. 10b, FIG. 12a-c, and Table 2). To gain insight into the binding of mAbs targeting the other bin, we obtained the cryo-EM structure of Fab 46 in complex with the RBD at a global resolution of 4.0 Å (FIG. 10c, FIG. 12d, and Table 2), and the crystal structure of Fabs 298 and 52 as a ternary complex with the RBD at 2.95 Å resolution (FIG. 10d, FIG. 2, and Table 3).

The crystal structure shows that Fab 298 binds almost exclusively to the ACE2 receptor binding motif (RBM) of the RBD (residues 438-506). In fact, out of 16 RBD residues involved in binding Fab 298, 12 are also involved in ACE2-RBD binding (FIG. 2a-c and Table 14). The RBM is stabilized by 11 hydrogen bonds from heavy and light chain residues of Fab 298. In addition, RBM Phe486 is contacted by 11 Fab 298 residues burying ˜170 Å² (24% of the total buried surface area on RBD) and hence is central to the antibody-antigen interaction (FIG. 2a and Table 14).

Detailed analysis of the RBD-52 Fab interface reveals that the epitope of mAb 52 is shifted towards the core of the RBD encompassing 20 residues of the RBM and seven residues in the core domain (FIG. 10c, FIG. 2b, and Table 14). In agreement with the competition data, antibody 52 and antibody 46 share a similar binding site, although they approach the RBD with slightly different angles (FIG. 10c, d and FIG. 2d). Inspection of previously reported structures of RBD-antibody complexes reveal that antibodies 46 and 52 target a site of vulnerability on the SARS-CoV-2 spike that has not been described previously (FIG. 10e). The epitope targeted by these antibodies is partially occluded by the NTD in the S “closed” conformation, suggesting that the mechanism of action for this class of antibodies could involve Spike destabilization. Together, these data demonstrate that the enhanced neutralization potency observed for the MB platform through avidity is associated with mAbs that can target distinct epitope bins on the RBD.

TABLE 14
RBD-298 and RBD-52 contacting residues identified by PISA

| | | BSA | | Fab 298 |
|---|---|---|---|---|
| RBD | Residue | (Å²) | Interaction | (H—HC, K—KC) |
| 453 | Tyr | 2 | vdW | H-Thr31, H-Ile100 |
| 455 | Leu | 20 | vdW | H-Thr31, H-Ile100 |
| 456 | Phe | 30 | vdW | H-Thr31, H-Tyr32 |
| 458 | Lys | 1 | vdW | K-Ser27F |
| 474 | Gln | 12 | vdW | K-Tyr27D |
| | Gln [symbol] | | HB | K-Tyr27D^{OH} |
| 475 | Ala | 45 | vdW | K-Tyr27D, H-Tyr32, |
| | | | | H-Arg97 |
| | Ala^{O} | | HB | K-Tyr32^{OH} |
| 476 | Gly | 23 | vdW | K-Tyr27D, K-Tyr32, |
| | | | | K-Tyr91, K-Tyr92, |
| | | | | K-Ser93, H-Arg97 |
| 477 | Ser | 75 | vdW | K-Tyr27D, K-Tyr92, |
| | | | | K-Ser93, K-Thr94 |
| | Ser^{N} | | HB | K-Tyr92^{O} |
| | Ser^{OG} | | HB | K-Tyr92^{O} |
| 478 | Thr | 41 | vdW | K-Tyr27D, K-Tyr92, |
| | | | | K-Ser93, K-Thr94 |
| | Thr^{OG} [symbol] | | HB | K-Tyr94^{OG} [symbol] |
| 484 | Glu | 74 | vdW | H-Trp50, H-Ser52, |
| | | | | H-Ser54, H-Gly55, |
| | | | | H-Gly56, H-Thr57, |
| | | | | H-Asp58 |
| | Glu^{OE2} | | HB | H-Ser54^{OG} |
| 485 | Gly | 28 | vdW | H-Trp50, H-Thr57, |
| | | | | H-Asp58 |
| 486 | Phe | 169 | vdW | K-Gln89, K-Tyr91, |
| | | | | K-Ser93, K-Tyr94, |
| | | | | K-Pro96, H-Ser35, |
| | | | | H-Trp47, H-Trp50, |
| | | | | H-Asp58, H-Asp95, |
| | | | | H-Arg97 |
| | Phe^{N} | | HB | H-Asp58 [symbol] |
| | Phe^{O} | | HB | K-Thr94^{OG} [symbol] |
| 487 | Asn | 41 | vdW | K-Tyr32, K-Tyr91, |
| | | | | K-Ser93, K-Thr94, |
| | | | | H-Asp95, H-Arg97 |
| | Asn^{OD1} | | HB | H-Arg97^{NH2} |
| | Asn^{ND2} | | HB | K-Tyr91^{O}, K-Tyr92^{O} |
| 488 | Cys | 1 | vdW | H-Trp50 |
| 489 | Tyr | 84 | vdW | H-Ser30, H-Thr31, |
| | | | | H-Tyr32, H-Trp50, |
| | | | | H-Asp95, H-Srg97, |
| | Tyr^{OH} | | HB | H-Arg97^{NH2}, H-Asp95^{OD2} |
| 493 | Gln | 45 | vdW | H-Ser30, H-Thr31, |
| | | | | H-Ile100 |
| Total | | 691 | | |
| BSA (Å²): | | | | |
| | | BSA | | Fab 298 |
| RBD | Residue | (Å²) | Interaction | (H—HC, K—KC) |
| 346 | Arg | 45 | vdW | H-Gln64 |
| 351 | Tyr | 29 | vdW | K-Phe94, H-Ile52, |
| | | | | H-Thr56, H-Asn58 |
| 352 | Ala | 19 | vdW | K-Gly93, K-Phe94 |
| 354 | Asn | 6 | vdW | K-Phe94 |
| 355 | Arg | 36 | vdW | K-Ser0, K-Gln27 |
| | Arg^{O} | | HB | K-Ser0^{OG} |
| 356 | Lys | 13 | vdW | K-Ser0 |
| 357 | Arg | 66 | vdW | K-Ser0, K-Gln27 |
| | Arg^{N} | | HB | K-Ser0^{OG} |
| | Arg^{NH2} | | HB | K-Gln27^{OE1} |
| 449 | Tyr | 13 | bdW | H-Phe554, H-Thr56 |
| 450 | Asn | 38 | vdW | H-Phe554, H-Thr56 |
| 452 | Leu | 47 | vdW | H-Ile52, H-Phe554, |
| | | | | H-Gly55, H-Thr56 |
| 462 | Lys | 1 | vdW | K-Ser30 |
| 465 | Glu | 28 | vdW | K-Ser30, K-Asn31, |
| | | | | K-Asn32, K-Asn92 |
| | Glu^{OE2} | | HB | K-Ser30^{OG} |
| 466 | Arg | 70 | vdW | K-Asn32, K-Asn92, |
| | | | | K-93Gly, K-Phe94 |
| | Arg^{O} | | HB | K-Asn32^{ND2} |
| | Arg^{NH} [symbol] | | HB | K-Asn92^{O} |
| 467 | Asp | 12 | vdW | K-Asn32, K-Asn92, |
| | | | | H-Asp98 |
| 468 | Ile | 102 | vdW | K-Asn32, K-Gly91, |
| | | | | K-Asn92, K-93Gly, |
| | | | | K-Phe94, K-Leu96, |
| | | | | H-Arg96, H-Gly97, |
| | | | | H-Asp98 |
| 469 | Ser | 37 | vdW | K-Asn32, H-Arg96, |
| | | | | H-Gly97, H-Asp98 |
| | Ser^{OG} | | HB | H-Asp98^{OD1} |
| 470 | Thr | 67 | vdW | Hp-Ser31, H-Tyr32, |
| | | | | H-Gly33, H-Ile52, |
| | | | | H-Asp95, H-Arg96, |
| | | | | H-Gly97, H-Asp98 |
| | Thr^{OG1} | | HB | H-Gly97^{N} |
| 471 | Glu | 39 | vdW | H-Tyr32, H-Arg96, |
| | | | | H-Gly97, H-Asp98 |
| | Glu^{OE1} | | SB | H-Arg96NH3 |
| | Glu^{OE2} | | SB | H-Arg96NH2 |
| 472 | Ile | 7 | vdW | H-Ser31, H-Tyr32, |
| | | | | H-Arg96 |
| 481 | Asn | 1 | vdW | H-Tyr32 |
| 482 | Gly | 42 | vdW | H-Ser31, H-Tyr32 |
| | Gly^{O} | | HB | H-Tyr^{OH} |
| 483 | Val | 12 | vdW | H-Thr28, H-Ser31, |
| | | | | H-Tyr32 |
| 484 | Glu | 53 | vdW | H-Thr28, H-Phe29, |
| | | | | H-Thr30, H-Ser31, |
| | | | | H-Met54 |
| 490 | Phe | 87 | vdW | H-Thr30, H-Ser31, |
| | | | | H-Tyr32, H-Gly33, |
| | | | | H-Ile52, H-Met54, |
| | | | | H-Phe554 |
| 492 | Leu | 13 | vdW | H-Ile52, H-Met54, |
| | | | | H-Phe554 |
| 493 | Gln | 8 | vdW | H-Phe554 |
| 494 | Ser | 17 | vdW | H-Phe554 |
| Total | | 904 | | |
| BSA (Å²): | | | | |
| | Residue- | BSA | | |
| Fab 298 | Chain | (Å²) | Interaction | RBD |
| 30 | Ser-H | 9 | vdW | Tyr489, Gln493 |
| 31 | Thr-H | 68 | vdW | Tyr453, Leu455, Phe456, |
| | | | | Tyr489, Gln493 |
| 32 | Tyr-H | 57 | vdW | Phe456, Ala475, Tyr489 |
| 35 | Ser-H | 8 | vdW | Phe486 |
| 47 | Trp-H | 23 | vdW | Phe486 |
| 50 | Trp-H | 79 | vdW | Glu484, Gly485, Phe486, |
| | | | | Cys488, Tyr489 |
| 52 | Ser-H | 8 | vdW | Glu484 |
| 54 | Ser-H | 17 | vdW | Glu484 |
| | Ser^{OG}-H | | HB | Glu484^{OE2} |
| 55 | Gly-H | 7 | vdW | Glu484 |
| 56 | Gly-H | 7 | vdW | Glu484 |
| 57 | Thr-H | 10 | vdW | Glu484, Gly485 |
| 58 | Asp-H | 28 | vdW | Glu484, Gly485, Phe486 |
| | Asp^{OD2}-H | | HB | Phe486^{N} |
| 95 | Asp-H | 15 | vdW | Phe486, Asn487, Tyr489 |
| | Asp^{OD2}-H | | HB | Tyr489^{OE} |
| 97 | Arg-H | 61 | vdW | Asn487, Tyr489 |
| | Arg^{NH2}-H | | HB | Asn487^{OD1}, Tyr489^{OH} |
| 100 | Ile-H | 7 | vdW | Tyr453, Leu455, Gln493 |
| 27D | Tyr-K | 59 | vdW | Gln474, Ala475, Gly476, |
| | | | | Ser477, Thr478 |
| | Tyr^{OH}-K | | HB | Gln474^{NE2} |
| 27F | Ser-K | 1 | vdW | Lys458 |
| 32 | Tyr-K | 28 | vdW | Ala475, Gly476, Asn487 |
| | Tyr^{OB}-K | | HB | Ala475^{O} |
| 89 | Gln-K | 1 | vdW | Phe486 |
| 91 | Tyr-K | 30 | vdW | GLy476, Phe486, Asn487 |
| | Tyr^{O}-K | | HB | Asn487^{ND2} |
| 92 | Tyr-K | 40 | vdW | Gly476, Ser477, Thr478 |
| | Tyr^{O}-K | | HB | Asn487^{ND2} |
| | Tyr^{O}-K | | HB | Ser477^{N}, Ser477^{OG} |
| 93 | Ser-K | 26 | vdW | Gly476, Ser477, Thr478, |
| | | | | Phe486, Ser487 |
| 94 | Thr-K | 57 | vdW | Ser477, Thr478, Phe486, |
| | | | | Asn487 |
| | Thr^{OG1}-K | | HB | Thr478^{OG1}, Phe486^{O} |
| 96 | Pro-K | 23 | vdW | Phe486 |
| Total | | | 669 | |
| BSA (Å²): | | | | |
| | Residue- | BSA | | |
| Fab 52 | Chain | (Å²) | Interaction | RBD |
| 28 | Thr-H | 24 | vdW | Val483, Glu484 |
| 30 | Thr-H | 3 | vdW | Glu484, Phe490 |
| 31 | Ser-H | 66 | vdW | Glu471, Gly482, |
| | | | | Val483, Glu484, |
| | | | | Phe490 |
| 32 | Tyr-H | 51 | vdW | Thr470, Glu471, Ile472, |
| | | | | Asn481, Gly482, Val483, |
| | | | | Phe490 |
| | Tyr^{OH}-H | | HB | Gly482^{O} |
| 33 | Gly-H | 7 | vdW | Thr470, Phe490 |
| 52 | Ile-H | 62 | vdW | Tyr351, Leu452, Thr470, |
| | | | | Phe490, Leu492 |
| 53 | Met-H | 38 | vdW | Glu484, Phe490, Leu492 |
| 54 | Phe-H | 110 | vdW | Tyr449, Asn450, Leu452, |
| | | | | Phe490, Leu492, Gln493, |
| | | | | Ser494 |
| 55 | Gly-H | 4 | vdW | Leu452 |
| 56 | Thr-H | 57 | vdW | Tyr351, Tyr449, Asn450, |
| | | | | Leu452 |
| 58 | Asn-H | 10 | vdW | Tyr351 |
| 64 | Gln-H | 43 | vdW | Arg346 |
| 95 | Asp-H | 2 | vdW | Thr470 |
| 96 | Arg-H | 64 | vdW | Ile468, Ser469, Thr470, |
| | | | | Glu471, Ile472 |
| | Arg^{NH2}-H | | SB | Glu471^{OE1}, Glu471^{OE2} |
| 97 | Gly-H | 64 | vdW | Ile468, Ser469, Thr470, |
| | | | | Glu471 |
| | Gly^{N}-H | | HB | Thr70^{OG1} |
| 98 | Asp-H | 28 | vdW | Asp467, Ile468, Ser469, |
| | | | | Thr470, Glu471 |
| | Asp^{OD1}-H | | HB | Ser469^{OG} |
| 0 | Ser-K | 77 | vdW | Arg355, Lys356, Arg357 |
| | Ser^{OG}-K | | HB | Arg355^{O}, Arg357^{N} |
| 27 | Gln-H | 60 | vdW | Arg355, Arg357 |
| | Gln^{OE3}-K | | HB | Arg357^{NH2} |
| 30 | Ser-K | 28 | vdW | Lys462, Glu465 |
| | Ser^{OG}-K | | HB | Glu465^{OE2} |
| 32 | Asn-K | 19 | vdW | Glu465, Arg466, Asp467, |
| | | | | Ile468, Ser469 |
| | Asn-^{ND2}-K | | HB | Arg466^{O} |
| 91 | Gly-K | 18 | vdW | Ile468 |
| 92 | Asn-K | 56 | vdW | Glu465, Arg466, Asp467, |
| | | | | Ile468 |
| | AsnO-K | | HB | Arg466NH1 |
| 93 | Gly-K | 22 | vdW | Ala352, Arg466, Ile468 |
| 94 | Phe-K | 56 | vdW | Tyr351, Ala352, Asn354, |
| | | | | Arg466, Ile468 |
| 96 | Leu-K | 5 | vdW | Ile468 |
| Total | | | 974 | |
| BSA (Å²): | | | | |

vdW: van der Waals interaction (5.0 Å cut-off)
HB: hydrogen bond (3.8 Å cut-off)
SB: salt bridge (4.0 Å cut-off) [symbol]
indicates data missing or illegible when filed

### Multabodies Overcome Spike Sequence Variability

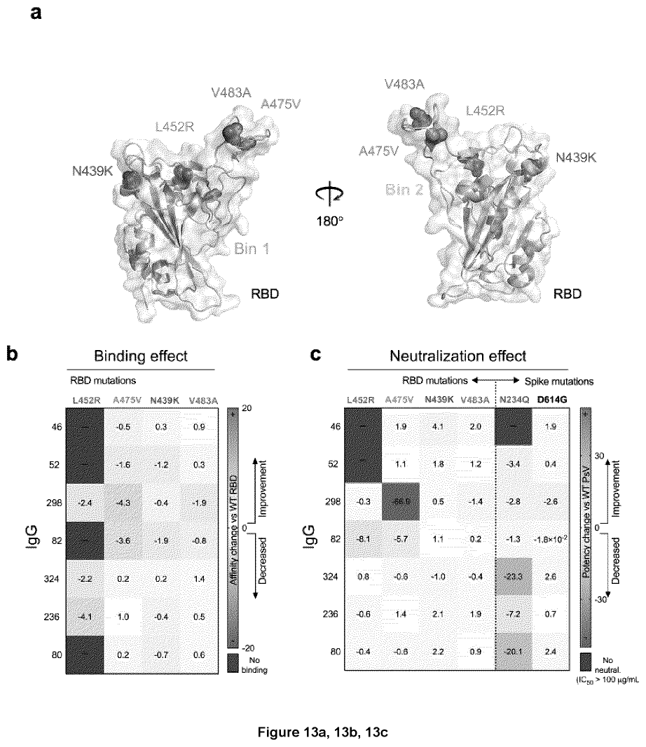

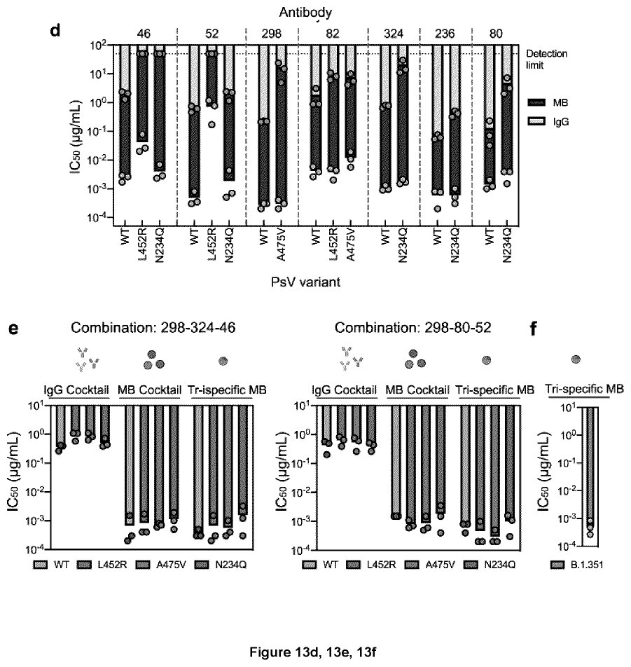

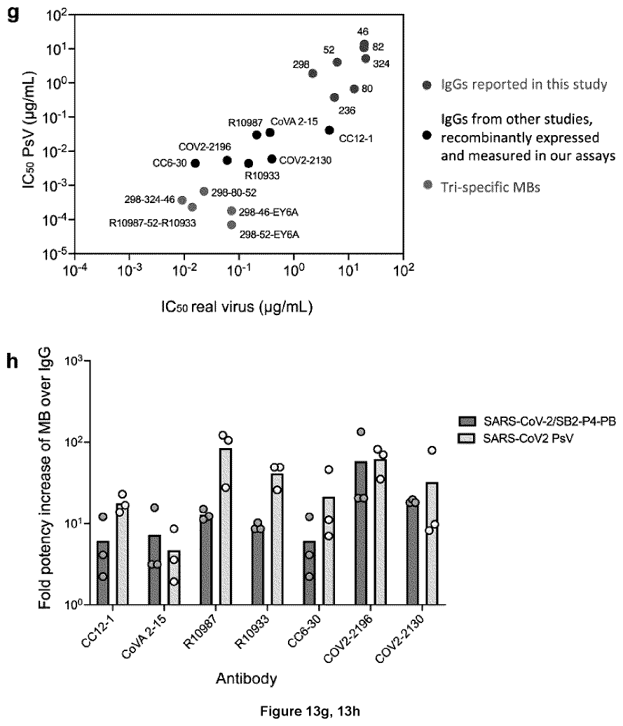

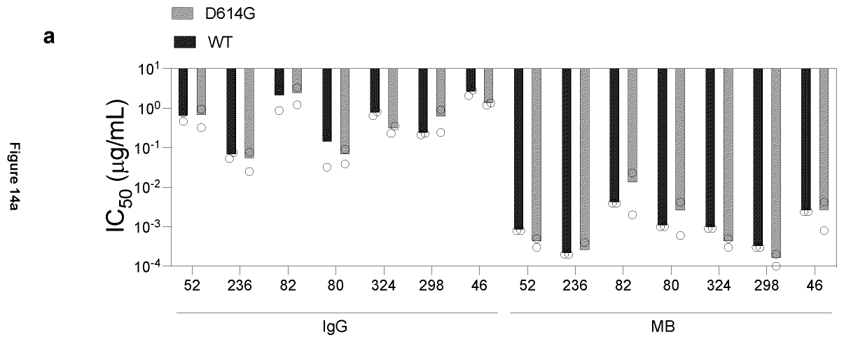

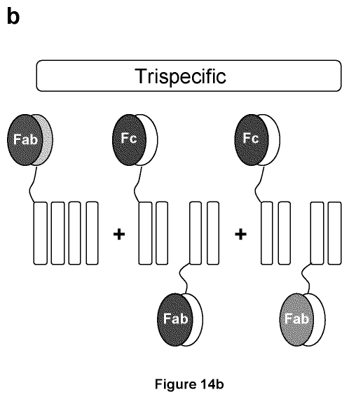

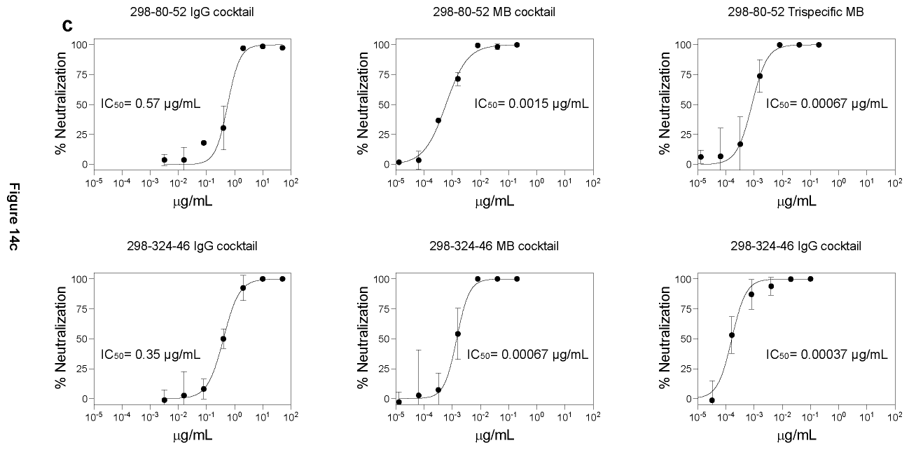

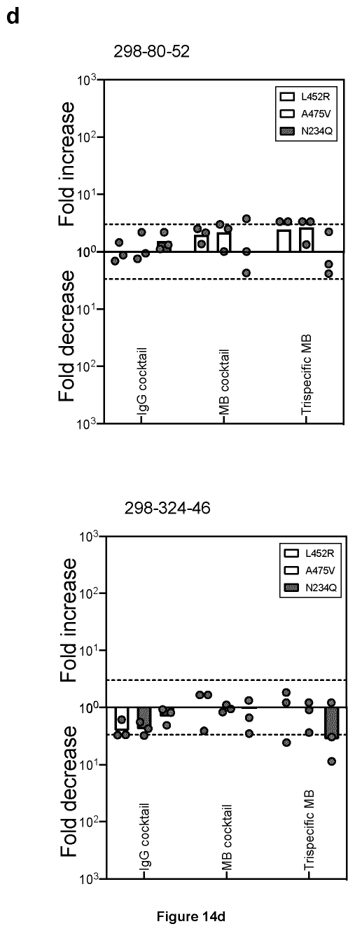

To explore whether MBs could potentially resist viral escape via their enhanced binding avidity, we tested the effect of four naturally occurring RBD mutations³⁵ on the binding and neutralization of the seven human mAbs of highest potency: L452R—located within the epitope of antibodies 46 and 52 (bin 1), A475V and V483A—located within the ACE2 binding site (bin 2), and the circulating RBD variant N439K³⁶ (FIG. 13a-c). In addition, the impact of mutating Asn234—an N-linked glycosylation site—to Gln was also assessed because the absence of glycosylation at this site has been previously reported to decrease sensitivity to neutralizing antibodies targeting the RBD³⁵. The more infectious PsV variant D614G³⁷ was also included in the panel. As expected, mutation L452R significantly decreased binding and potency of mAbs 52 and 46, while antibody 298 was sensitive to mutation A475V (FIG. 13b, c). Deletion of the N-linked glycan at position Asn234 increased viral resistance to the majority of the antibodies, especially mAbs 46, 80, and 324, emphasizing the importance of glycans in viral antigenicity (FIG. 13c). Strikingly, the following antibody specificities in the MB format were minimally impacted in their exceptional neutralization potency by any S mutation: 298, 80, 324, and 236 (FIG. 13d). Mutation L452R decreased the sensitivity of the 46-MB and 52-MB but in contrast to their parental IgGs, they remained neutralizing against this PsV variant (FIG. 13d). The more infectious SARS-CoV-2 PsV variant D614G was neutralized with similar potency as the WT PsV for both IgGs and MBs (FIG. 13c and FIG. 14a).

MB cocktails consisting of three monospecific MBs resulted in pan-neutralization across all PsV variants without a significant loss in potency and hence achieved a 100-1000-fold higher potency compared to the corresponding IgG cocktails (FIG. 13e and FIG. 14c, d). In order to achieve breadth within a single molecule, we next generated tri-specific MBs by combining multimerization subunits displaying three different Fabs in the same MB assembly (FIG. 14b). Notably, the resulting tri-specific MBs exhibited pan-neutralization while preserving the exceptional neutralization potency of the monospecific versions including against the B.1.351 PsV variant (FIG. 13e, f and FIG. 14c, d). The highest potency was observed for the 298-324-46 combination (FIG. 14c, e), where the tri-specific MB achieved exceptional potency beyond that observed for some of the most potent IgGs reported to date and that we generated recombinantly from available sequences (FIG. 13g). In addition, the MB format was able to increase the potency of these previously reported highly potent IgGs by a further one to two orders of magnitude against PsV and live replicating SARS-CoV-2 virus (FIG. 13h), thus highlighting the plug-and-play nature of the MB and the ability of multivalency to enhance the neutralization capacity of mAbs across a range of potencies.

The values determined for median IC₅₀ of neutralization are summarized in Table 13 and Table 15.

TABLE 15
SARS-CoV-2 Neutralization by Multabodies

| Multabody | Median IC₅₀ | |
|---|---|---|
| VHH-hFerr | 0.00011 | μg/mL (0.13 pM) |
| VHH-Fc | 1.3 | μg/mL (16 nM) |
| BD23 MB | 0.008 | μg/mL (3.5 pM) |
| BD23 IgG | 13 | μg/mL (87 nM) |
| 4A8 MB | 0.052 | μg/mL (23 pM) |
| 4A8 IgG | >100 | g/mL (> 0.66 μM) |

### Discussion

In this study, we reveal how binding avidity can be leveraged as an effective mechanism to propel antibody neutralization potency and resistance from viral mutations. To this effect, we used protein engineering to develop a plug-and-play antibody-multimerization platform that increases avidity of mAbs targeting SARS-CoV-2. The seven most potent MBs have IC₅₀ values of 0.2 to 2 ng/mL (9×10⁻¹⁴ to 9×10⁻¹³ M) against SARS-CoV-2 PsVs and therefore are, to our knowledge, within the most potent antibody-like molecules reported to date against SARS-CoV-2.

The MB platform was designed to include key favorable attributes from a developability perspective. First, the ability to augment antibody potency is independent of antibody sequence, format or epitope targeted. The modularity and flexibility of the platform was exemplified by enhancing the potency of a VHH and multiple Fabs that target non-overlapping regions on two SARS-CoV-2 S sub-domains (RBD and NTD). Using the MB to enhance the potency of VHH domains could provide particular value to this class of molecules since its small size allows highly efficient multimerization. Second, in contrast to other approaches that enhance avidity through tandem fusions of single chain variable fragments^{38,39}, MBs do not suffer from low stability and in fact self-assemble into highly stable particles with aggregation temperatures similar to those of their parental IgGs. Third, alternative multimerization strategies like streptavidin⁴⁰, verotoxin B subunit scaffolds⁴¹, or viral-like nanoparticles⁴² face immunogenicity challenges and/or poor bioavailability because of the absence of a Fc fragment and therefore the inability to undergo FcRn-mediated recycling. The light chain of apoferritin is fully human, biologically inactive, has been engineered to include Fc domains, and despite multimerization of >24 Fab/Fc fragments, has a Rh similar to an IgM. As such, a surrogate mouse MB did not elicit antidrug antibodies in mice and similar to its parental IgG was detectable in the sera for over a week. However, in vivo bioavailability of the MB was dependent on its binding affinity to FcγRs, suggesting that Fc avidity will need to be carefully fine-tuned for efficient translation of the MB to the clinic. In addition, further studies will be needed to evaluate how the MB distributes at anatomical sites of interest, such as the lungs in the case of SARS-CoV-2 infection. The plug-and-play nature of the Multabody also lends itself to exploring alternate half-life extending moieties other than the Fc if bioavailability is the only desired trait absent of effector functions e.g., human serum albumin⁴³, or binding moieties that bind human serum albumin^{44,45}.

Different increases in neutralization potency were observed for different mAb sequences tested on the MB against SARS-CoV-2. This suggests that the ability of the MB to enhance potency may depend on epitope location on the Spike, or the geometry of how the Fabs engage the antigen to achieve neutralization. The fact that the neutralization of two out of 20 SARS-CoV-2 RBD binders were not rescued by the MB platform suggests limitations based on mAb sequences and binding properties alone. Nevertheless, the capacity of the MB to transform avidity into neutralization potency across a range of epitope specificities on the SARS-CoV-2 Spike highlights the potential for using this technology broadly. It will be interesting to explore the potency-enhancement capacity of the MB platform against viruses with low surface spike density like HIV-1⁴⁶, or against other targets like the tumor necrosis factor receptor superfamily, where bivalency of conventional antibodies limits their efficient activation⁴⁷.

Virus escape can arise in response to selective pressure from treatments or during natural selection. A conventional approach to combat escape mutants is the use of antibody cocktails targeting different epitopes. MBs showed a lower susceptibility to S mutations in comparison to their parental IgGs, presumably because the loss in affinity was compensated by enhanced binding avidity. Hence, when used in cocktails, the MB overcame viral sequence variability with exceptional potency. In addition, the split MB design allows combination of multiple antibody specificities within a single multimerized molecule resulting in similar potency and breadth as the MB cocktails. Importantly, the B.1.351 variant of concern that can escape the neutralization of several mAbs^{21,22,23} is neutralized with high potency by a tri-specific Multabody, thus further highlighting the capacity of these molecules to resist viral escape. Multi-specificity within the same particle could offer additional advantages such as intra-S avidity and synergy for the right combination of mAbs, setting the stage for further investigation of different combinations of mAb specificities on the MB. Avidity and multi-specificity could also be leveraged to deliver a single molecule that neutralizes potently across viral genera, such as betacoronaviruses.

Overall, the MB platform provides a tool to surpass antibody affinity limits and generate broad and potent neutralizing molecules while by-passing extensive antibody discovery or engineering efforts. This platform is an example of how binding avidity can be leveraged to accelerate the timeline to discovery of the most potent biologics against infectious diseases of global health importance.

### REFERENCES

1. Connor, E. M. Palivizumab, a humanized respiratory syncytial virus monoclonal antibody, reduces hospitalization from respiratory syncytial virus infection in high-risk infants. Pediatrics 102, 531-537 (1998).
2. Mulangu, S. et al. A randomized, controlled trial of Ebola virus disease therapeutics. N. Engl. J. Med. 381, 2293-2303 (2019).
3. Ju, B. et al. Human neutralizing antibodies elicited by SARS-CoV-2 infection. Nature 584, 115-119 (2020).
4. Liu, L. et al. Potent neutralizing antibodies against multiple epitopes on SARS-CoV-2 spike. Nature 584, 450-456 (2020).
5. Wang, C. et al. A human monoclonal antibody blocking SARS-CoV-2 infection. Nat. Commun. 11, 2251 (2020).
6. Zost, S. J. et al. Potently neutralizing and protective human antibodies against SARS-CoV-2. Nature 584, 443-449 (2020).
7. Baum, A. et al. Antibody cocktail to SARS-CoV-2 spike protein prevents rapid mutational escape seen with individual antibodies. Science 369, 1014-1018 (2020).
8. Hansen, J. et al. Studies in humanized mice and convalescent humans yield a SARS-CoV-2 antibody cocktail. Science 369, 1010-1014 (2020).
9. Lv, Z. et al. Structural basis for neutralization of SARS-CoV-2 and SARS-CoV by a potent therapeutic antibody. Science 369, 1505-1509 (2020).
10. Tortorici, M. A. et al. Ultrapotent human antibodies protect against SARS-CoV-2 challenge via multiple mechanisms. Science 370, 950-957 (2020).
11. Zhou, D. et al. Structural basis for the neutralization of SARS-CoV-2 by an antibody from a convalescent patient. Nat. Struct. Mol. Biol. 27, 950-958 (2020).
12. Cao, Y. et al. Potent neutralizing antibodies against SARS-CoV-2 identified by high-throughput single-cell sequencing of convalescent patients' B cells. Cell 182, 73-84 (2020).
13. Chi, X. et al. A neutralizing human antibody binds to the N-terminal domain of the Spike protein of SARS-CoV-2. Science 369, 650-655 (2020).
14. Seydoux, E. et al. Analysis of a SARS-CoV-2-infected individual reveals development of potent neutralizing antibodies with limited somatic mutation. Immunity 53, 98-105 (2020).
15. Pinto, D. et al. Cross-neutralization of SARS-CoV-2 by a human monoclonal SARS-CoV antibody. Nature 583, 290-295 (2020).
16. Barnes, C. O. et al. Structures of human antibodies bound to SARS-CoV-2 spike reveal common epitopes and recurrent features of antibodies. Cell 182, 828-842.e16 (2020).
17. Shi, R. et al. A human neutralizing antibody targets the receptor-binding site of SARS-CoV-2. Nature 584, 120-124 (2020).
18. Rogers, T. F. et al. Isolation of potent SARS-CoV-2 neutralizing antibodies and protection from disease in a small animal model. Science 369, 956-963 (2020).
19. Brouwer, P. J. M. et al. Potent neutralizing antibodies from COVID-19 patients define multiple targets of vulnerability. Science 369, 643-650 (2020).
20. Hoffmann, M. et al. SARS-CoV-2 cell entry depends on ACE2 and TMPRSS2 and is blocked by a clinically proven protease inhibitor. Cell 181, 271-280 (2020).
21. Wang, P. et al. Increased resistance of SARS-CoV-2 variant P.1 to antibody neutralization. Cell Host Microbe. 29, 747-751.e4 (2021).
22. Wu, K. et al. mRNA-1273 vaccine induces neutralizing antibodies against spike mutants from global SARS-CoV-2 variants. Preprint at bioRxiv (2021).
23. Wibmer, C. K. et al. SARS-CoV-2 501Y.V2 escapes neutralization by South African COVID-19 donor plasma. Nat. Med. 27, 622-625 (2021).
24. Rambaut, A. et al. Preliminary genomic characterisation of an emergent SARS-CoV-2 lineage in the UK defined by a novel set of spike mutations. Preprint at https://virological.org/t/preliminarygenomic-characterisation-of-an-emergent-sars-cov-2-lineage-in-the-uk-defined-by-a-novel-set-ofspike-mutations/563 (2020).
25. Tegally, H. et al. Emergence and rapid spread of a new severe acute respiratory syndrome-related coronavirus 2 (SARS-CoV-2) lineage with multiple spike mutations in South Africa. Preprint at medRxiv https://doi.org/10.1101/2020.12.21.20248640 (2020).
26. Faria, N. R. et al. Genomics and epidemiology of the P.1 SARS-CoV-2 lineage in Manaus, Brazil. Science. 372, 815-821 (2021).
27. Naveca, F. et al. Phylogenetic relationship of SARS-CoV-2 sequences from Amazonas with emerging Brazilian variants harboring mutations E484K and N501Y in the spike protein. Preprint at https://virological.org/t/phylogenetic-relationship-of-sars-cov-2-sequences-from-amazonas-with-emerging-brazilian-variants-harboring-mutations-e484k-and-n501y-in-the-spike-protein/585 (2021).
28. Wu, H. et al. Ultra-potent antibodies against respiratory syncytial virus: effects of binding kinetics and binding valence on viral neutralization. J. Mol. Biol. 350, 126-144 (2005).
29. Icenogle, J. et al. Neutralization of poliovirus by a monoclonal antibody: kinetics and stoichiometry. Virology 127, 412-425 (1983).
30. Cavacini, L. A., Emes, C. L., Power, J., Duval, M. & Posner, M. R. Effect of antibody valency on interaction with cell-surface expressed HIV-1 and viral neutralization. J. Immunol. 152, 2538-2545 (1994).
31. Wrapp, D. et al. Structural basis for potent neutralization of betacoronaviruses by single-domain camelid antibodies. Cell 181, 1004-1015.e15 (2020).
32. Li, W. et al. High potency of a bivalent human VH domain in SARS-CoV-2 animal models. Cell 183, 1-13 (2020).
33. Lawson, D. M. et al. Solving the structure of human H ferritin by genetically engineering intermolecular crystal contacts. Nature 349, 541-544 (1991).
34. Radomsky, M. L., Whaley, K. J., Cone, R. A. & Saltzman, W. M. Macromolecules released from polymers: diffusion into unstirred fluids. Biomaterials 11, 619-624 (1990).
35. Li, Q. et al. The impact of mutations in SARS-CoV-2 spike on viral infectivity and antigenicity. Cell 182, 1284-1294 (2020).
36. Thomson, E. C. et al. Circulating SARS-CoV-2 spike N439K variants maintain fitness while evading antibody-mediated immunity. Cell. 185, 1171-1187.e20 (2021).
37. Korber, B. et al. Tracking changes in SARS-CoV-2 spike: evidence that D614G increases infectivity of the COVID-19 virus. Cell 182, 812-827 (2020).
38. Miller, K. et al. Design, construction, and in vitro analyses of multivalent antibodies. J. Immunol. 170, 4854-4861 (2003).
39. Kipriyanov, S. M. et al. Bispecific tandem diabody for tumor therapy with improved antigen binding and pharmacokinetics. J. Mol. Biol. 293, 41-56 (1999).
40. Kipriyanov, S. M. et al. Affinity enhancement of a recombinant antibody: formation of complexes with multiple valency by a single-chain Fv fragment-core streptavidin fusion. Protein Eng. 9, 203-211 (1996).
41. Zhang, J. et al. Pentamerization of single-domain antibodies from phage libraries: a novel strategy for the rapid generation of high-avidity antibody reagents. J. Mol. Biol. 335, 49-56 (2004).
42. Hoffmann, M. A. G. et al. Nanoparticles presenting clusters of CD4 expose a universal vulnerability of HIV-1 by mimicking target cells. Proc. —Natl Acad. Sci. USA 117, 18719-18728 (2020).
43. Sleep, D., Cameron, J. & Evans, L. R. Albumin as a versatile platform for drug half-life extension. Biochim. Biophys. Acta 1830, 5526-5534 (2013).
44. van Faassen, H. et al. Serum albumin-binding VHHs with variable pH sensitivities enable tailored half-life extension of biologics. FASEB J. 34, 8155-8171 (2020).
45. Adams, R. et al. Extending the half-life of a fab fragment through generation of a humanized anti-human serum albumin Fv domain: an investigation into the correlation between affinity and serum half-life. MAbs 8, 1336-1346 (2016).
46. Zhu, P. et al. Electron tomography analysis of envelope glycoprotein trimers on HIV and simian immunodeficiency virus virions. Proc. Natl Acad. Sci. USA 100, 15812-15817 (2003).
47. Wajant, H. Principles of antibody-mediated TNF receptor activation. Cell Death Differ. 22, 1727-1741 (2015).
48. Schlothauer, T. et al. Novel human IgG1 and IgG4 Fc-engineered antibodies with completely abolished immune effector functions. Protein Eng. Des. Sel. 29, 457-466 (2016).
49. Crawford, K. H. D. et al. Protocol and reagents for pseudotyping lentiviral particles with SARS-CoV-2 spike protein for neutralization assays. Viruses 12, 513 (2020).
50. Banerjee, A. et al. Isolation, sequence, infectivity, and replication kinetics of severe acute respiratory syndrome coronavirus 2. Emerg. Infect. Dis. 26, 2054-2063 (2020).
51. Marr, C. R., Benlekbir, S. & Rubinstein, J. L. Fabrication of carbon films with ˜500 nm holes for cryo-EM with a direct detector device. J. Struct. Biol. 185, 42-47 (2014).
52. Punjani, A., Rubinstein, J. L., Fleet, D. J. & Brubaker, M. A. CryoSPARC: algorithms for rapid unsupervised cryo-EM structure determination. Nat. Methods 14, 290-296 (2017).
53. Zi Tan, Y. et al. Addressing preferred specimen orientation in single-particle cryo-EMthrough tilting. Nat. Methods 14, 793-796 (2017).
54. Zivanov, J. et al. New tools for automated high-resolution cryo-EM structure determination in RELION-3. Elife 9, e42166 (2018).
55. Scheres, S. H. W. RELION: implementation of a Bayesian approach to cryo-EM structure determination. J. Struct. Biol. 180, 519-530 (2012).
56. Punjani, A. & Fleet, D. J. 3D variability analysis: Resolving continuous flexibility and discrete heterogeneity from single particle cryo-EM. J. Struct. Biol. 213, 107702 (2021).
57. Punjani, A., Zhang, H. & Fleet, D. J. Non-uniform refinement: adaptive regularization improves single-particle cryo-EM reconstruction. Nat. Methods. 17, 1214-1221 (2020).
58. Grant, T., Rohou, A. & Grigorieff, N. CisTEM, user-friendly software for single-particle image processing. Elife 7, e35383 (2018).
59. Asarnow, D., Palovcak, E. & Cheng, Y. asarnow/pyem: UCSF pyem v0. 5 (Zenodo, 2019).
60. Kabsch, W. et al. XDS. Acta Crystallogr. D. Biol. Crystallogr. 66, 125-132 (2010).
61. McCoy, A. J. et al. Phaser crystallographic software. J. Appl. Crystallogr. 40, 658-674 (2007).
62. Adams, P. D. et al. PHENIX: a comprehensive Python-based system for macromolecular structure solution. Acta Crystallogr. D Biol. Crystallogr. 66, 213-221 (2010).
63. Emsley, P., Lohkamp, B., Scott, W. G. & Cowtan, K. Features and development of Coot. Acta Crystallogr. Sect. D Biol. Crystallogr. 66, 486-501 (2010).
64. The PyMol Molecular Graphics System, Versión 1.8 (Schrödinger, LLC., 2015).
65. Morin, A. et al. Collaboration gets the most out of software. Elife 2, e01456 (2013).
66. Lan, J. et al. Structure of the SARS-CoV-2 spike receptor-binding domain bound to the ACE2 receptor. Nature 581, 215-220 (2020).
67. Yuan, M. et al. A highly conserved cryptic epitope in the receptor binding domains of SARS-CoV-2 and SARS-CoV. Science 368, 630-633 (2020).
68. Piccoli, L. et al. Mapping neutralizing and immunodominant sites on the SARS-CoV-2 spike receptor-binding domain by structure-guided high-resolution serology. Cell 183, 1024-1042.e21 (2020).
69. Hurlburt, N. K. et al. Structural basis for potent neutralization of SARS-CoV-2 and role of antibody affinity maturation. Nat. Commun. 11, 5413 (2020).
70. Barnes, C. O. et al. SARS-CoV-2 neutralizing antibody structures inform therapeutic strategies. Nature 588, 682-687 (2020).
71. Yuan, M. et al. Structural basis of a shared antibody response to SARS-CoV-2. Science 369, 1119-1123 (2020).
72. Hansen, J. et al. Studies in humanized mice and convalescent humans yield a SARS-CoV-2 antibody cocktail. Science 369, 1010-1014 (2020).

### Example 2

### Virus Production and Pseudovirus Neutralization Assays

SARS-CoV-2 pseudotyped viruses (PsV) were generated using an HIV-based lentiviral system as previously described⁴⁴ with few modifications. Briefly, 293T cells were co-transfected with a lentiviral backbone encoding the luciferase reporter gene (BEI NR52516), a plasmid expressing the Spike (BEI NR52310) and plasmids encoding the HIV structural and regulatory proteins Tat (BEI NR52518), Gag-pol (BEI NR52517) and Rev (BEI NR52519). 24 h post transfection at 37° C., 5 mM sodium butyrate was added to the media and the cells were incubated for an additional 24-30 h at 30° C. PsV mutants were generated using the KOD-Plus mutagenesis kit (Toyobo, Osaka, Japan). SARS-CoV-2 spike variants of concern B.1.117, B.1.351, P.1 and B.1.617.2 were kindly provided by David Ho (Columbia). Neutralization was determined in a single-cycle neutralization assay using 293T-ACE2 cells (BEI NR52511). PsV neutralization was monitored by adding Britelite plus reagent (PerkinElmer) to the cells and measuring luminescence in relative light units (RLUs) using a Synergy Neo2 Multi-Mode Assay Microplate Reader (Biotek Instruments). IC₅₀ fold increase was calculated as: IgG_{IC50} (μg/mL)/MB_{IC50} (μg/mL). Two to three biological replicates with two technical replicates each were performed.

### Results

### Identification of Sequence Liability in mAb 52

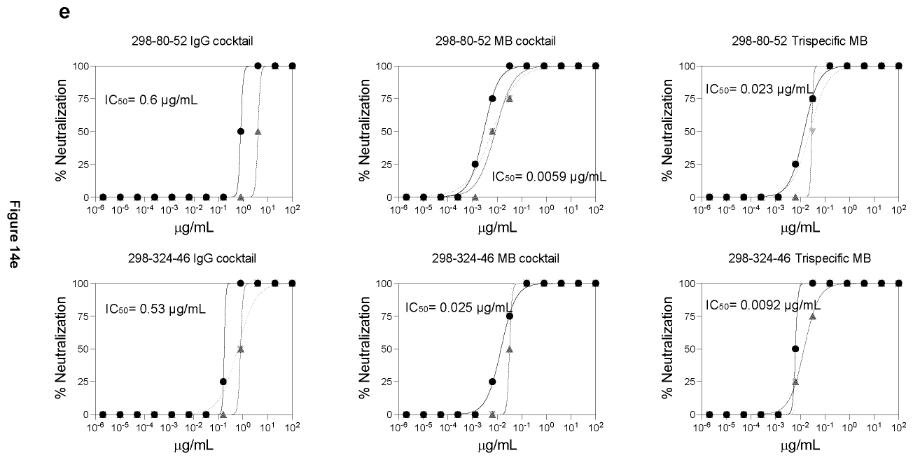

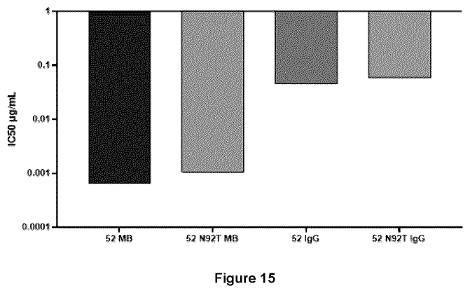

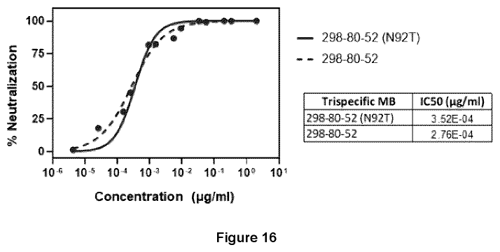

in silico analysis of lead VH/VL sequences identified a deamidation site in the CDRL3 of mAb52 at position N92. Deamidation sites in mAbs can contribute to both changes in binding kinetics and heterogeneity in the drug product. In order to circumvent this potential effect, we generated variants in which the asparagine residue was mutated to a threonine (N92T). FIG. 15 shows that this mutation did not have any effects in potency as both an IgG or as a monospecific MB in a WT pseudovirus neutralization assay. The 298-80-52 trispecific MB containing the N92T mutation in the VL of mAb 52 was subsequently screened in a P.1 PsV neutralization assay and the results confirmed that there was no loss in potency observed compared to the parental trispecific MB (FIG. 16).

### Neutralization of a Trispecific MB Across Variants of Concern

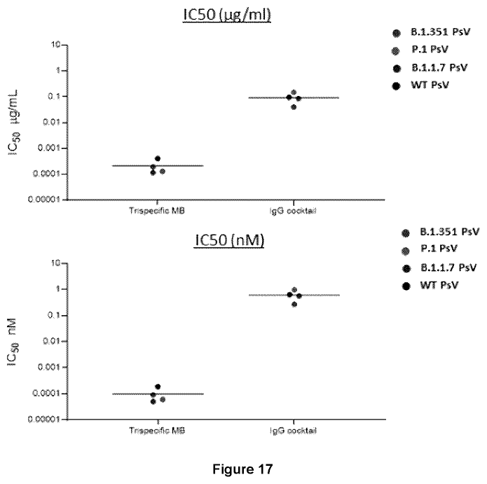

An example trispecific MB, 298-80-52, was assessed for potency across the variants of concern (VOCs) in pseudovirus neutralization assays. As shown in Table 16 and in FIG. 17, this MB retained activity across the VOCs with an average IC₅₀ from the WT, B.1.1.7, B.1.351 and P.1 PsV assays of approximately 0.2 ng/ml [97 fM], while the corresponding IgG cocktail had an average IC₅₀ of 91 ng/ml [0.61 nM](n=5 experiments). This represents an increase in potency of ˜200-1000 fold in ng/ml or ˜3000-16000 fold in molarity. These results highlight the ability of the trispecific MB to overcome viral escape of SARS-CoV-2 at exceptional potencies.

TABLE 16

| | Trispecific MB | IgG cocktail | Fold Increase in | Trispecific MB | IgG cocktail | Fold Increase in |
|---|---|---|---|---|---|---|
| PsV | IC50 (μg/ml) | | Potency | IC50 (μM) | | Potency |
| WT | 4.10E−04 | 9.52E−02 | 232 | 1.87E−04 | 6.34E−01 | 3401 |
| 8.1.351 | 1.94E−04 | 4.02E−02 | 207 | 8.82E−05 | 2.68E−01 | 3037 |
| 8.3.1.7 | 1.16E−04 | 8.51E−02 | 733 | 5.28E−05 | 5.67E−01 | 10749 |
| P.1 | 1.30E−04 | 1.47E−01 | 1130 | 5.92E−05 | 9.81E−01 | 16577 |
| Average | 2.13E−04 | 9.19E−02 | 576 | 9.67E−05 | 6.13E−01 | 8441 |

```
hFerritinLC
                                                                                                      SEQ ID NO:1
MSSQIRQNYSTDVEAAVNSLVNLYLQASYTYLSLGFYFDRDDVALEGVSHFFRELAEEKREG
YERLLKMQNQRGGRALFQDIKKPAEDEWGKTPDAMKAAMALEKKLNQALLDLHALGSARTDP
HLCDFLETHFLDEEVKLIKKMGDHLTNLHRLGGPEAGLGEYLFERLTLRHD
Linker1
                                                                                                      SEQ ID NO:2
GGGGSGGGGSGGGGSGGGGSGGGGSGG
VHH-hFerr
(Underlining indicates linker sequence; bolding indicates
hFerritinLC)
                                                                                                      SEQ ID NO:3
QVQLQESGGGLVQAGGSLRLSCAASGRTFSEYAMGWFRQAPGKEREFVATISWSGGSTYYTD
SVKGRFTISRDNAKNTVYLQMNSLKPDDTAVYYCAAAGLGTVVSEWDYDYDYWGQGTQVTVS
GGGGSGGGGSGGGGSGGGGSGGGGSGGGSSQIRQNYSTDVEAAVNSLVNLYLQASYTYLSLG
FYFDRDDVALEGVSHFFRELAEEKREGYERLLKMQNQRGGRALFQDIKKPAEDEWGKTPDAM
KAAMALEKKLNQALLDLHALGSARTDPHLCDFLETHFLDEEVKLIKKMGDHLTNLHRLGGPE
AGLGEYLFERLTLRHD
VHH-Fc
(Underlining indicates linker sequence)
                                                                                                      SEQ ID NO:4
QVQLQESGGGLVQAGGSLRLSCAASGRTFSEYAMGWFRQAPGKEREFVATISWSGGSTYYTD
SVKGRFTISRDNAKNTVYLQMNSLKPDDTAVYYCAAAGLGTVVSEWDYDYDYWGQGTQVTVS
SGSDKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK
N-hFerritinLC
                                                                                                      SEQ ID NO:5
MSSQIRQNYSTDVEAAVNSLVNLYLQASYTYLSLGFYFDRDDVALEGVSHFFRELAEEKREG
YERLLKMQNQRGGRALFQDIKKPAEDEW
C-hFerritinLC
                                                                                                      SEQ ID NO:6
GKTPDAMKAAMALEKKLNQALLDLHALGSARTDPHLCDFLETHFLDEEVKLIKKMGDHLTNL
HRLGGPEAGLGEYLFERLTLRHD
Signal sequence
                                                                                                      SEQ ID NO:7
MGILPSPGMPALLSLVSLLSVLLMGCVAE
Linker 1
                                                                                                      SEQ ID NO:8
GGGGSGGGGSGGGGSGGGGSGGGGSGGGGSGGGGSGGGGSGGGGSGGGGSGGGGSGGGGSGG
GGSGGGGS
Linker 2
                                                                                                      SEQ ID NO:9
GGGGSGGGGSGGGGSGGGGSGGGGSGG
BD23-scFab-hFerritinLC
(Underlining indicates linker sequence; bolding indicates
hFerritinLC)
                                                                                                    SEQ ID NO:10
DIQMTQSPSTLSASVGDRVTITCRASQSISSWLAWYQQKPGKAPKLLIYKASSLESGVPSRF
SGSGSGTEFTLTISSLQPDDFATYYCQQYNSYPYTFGQGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGECGGGGSGGGGSGGGGSGGGGSGGGGSGGGGSGGGG
SGGGGSGGGGSGGGGSGGGGSGGGGSGGGGSGGGGSQVQLVQSGSELKKPGASVKVSCKASG
YTFTSYAMNWVRQAPGQGLEWMGWINTNTGNPTYAQGFTGRFVFSLDTSVSTAYLQISSLKA
EDTAVYYCARPQGGSSWYRDYYYGMDVWGQGTTVTVSSASTKGPSVFPLAPSSKSTSGGTAA
LGCLVKDYFPEPVTVSWNSGALTSGVHTFPAVLQSSGLYSLSSWVTVPSSSLGTQTYICNVN
HKPSNTKVDKKVEPKSCGGGGSGGGGSGGGGSGGGGSGGGGSGGSSQIRQNYSTDVEAAVNS
LVNLYLQASYTYLSLGFYFDRDDVALEGVSHFFRELAEEKREGYERLLKMQNQRGGRALFQD
IKKPAEDEWGKTPDAMKAAMALEKKLNQALLDLHALGSARTDPHLCDFLETHFLDEEVKLIK
KMGDHLTNLHRLGGPEAGLGEYLFERLTLRHD
V_{K} for BD23
                                                                                                    SEQ ID NO:11
DIQMTQSPSTLSASVGDRVTITCRASQSISSWLAWYQQKPGKAPKLLIYKASSLESGVPSRF
SGSGSGTEFTLTISSLQPDDFATYYCQQYNSYPYTFGQGTKLEIK
V_{H} for BD23
                                                                                                    SEQ ID NO:12
QVQLVQSGSELKKPGASVKVSCKASGYTFTSYAMNWVRQAPGQGLEWMGWINTNTGNPTYAQ
GFTGRFVFSLDTSVSTAYLQISSLKAEDTAVYYCARPQGGSSWYRDYYYGMDVWGQGTTVTV
SS
BD23-scFab-C_hFerritinLC
(Underlining indicates linker sequence; bolding indicates
C_hFerritinLC)
                                                                                                    SEQ ID NO:13
LEDIQMTQSPSTLSASVGDRVTITCRASQSISSWLAWYQQKPGKAPKLLIYKASSLESGVPS
RFSGSGSGTEFTLTISSLQPDDFATYYCQQYNSYPYTFGQGTKLEIKRTVAAPSVFIFPPSD
EQLKSGTASVVCLLNNFYPREAKVQWKVDNALQSGNSQESVTEQDSKDSTYSLSSTLTLSKA
DYEKHKVYACEVTHQGLSSPVTKSFNRGECGGGGSGGGGSGGGGSGGGGSGGGGSGGGGSGG
GGSGGGGSGGGGSGGGGSGGGGSGGGGSGGGGSGGGGSQVQLVQSGSELKKPGASVKVSCKA
SGYTFTSYAMNWVRQAPGQGLEWMGWINTNTGNPTYAQGFTGRFVFSLDTSVSTAYLQISSL
KAEDTAVYYCARPQGGSSWYRDYYYGMDVWGQGTTVTVSSASTKGPSVFPLAPSSKSTSGGT
AALGCLVKDYFPEPVTVSWNSGALTSGVHTFPAVLQSSGLYSLSSVVTVPSSSLGTQTYICN
VNHKPSNTKVDKKVEPKSCGGGGSGGGGSGGGGSGGGGSGGGGSGGGKTPDAMKAAMALEKK
LNQALLDLHALGSARTDPHLCDFLETHFLDEEVKLIKKMGDHLTNLHRLGGPEAGLGEYLFE
RLTLRHD
scFc-N_hFerritinLC
(Underlining indicates linker sequence; bolding indicates
hFerritinLC)
                                                                                                    SEQ ID NO:14
DKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGKGGGGSGGGGSGGGGSGGGGSG
GGGSGGGGSGGGGSGGGGSGGGGSGGGGSGGGGSGGGGSGGGGSGGGGSDKTHTCPPCPAPE
LLGGPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFLYSKLTVDKSRWQQ
GNVFSCSVMHEALHNHYTQKSLSLSPGKGGGGSGGGGSGGGGSGGGGSGGGGSGGSSQIRQN
YSTDVEAAVNSLVNLYLQASYTYLSLGFYFDRDDVALEGVSHFFRELAEEKREGYERLLKMQ
NQRGGRALFQDIKKPAEDEW
scFc(LALAP)
(Residue(s) that are mutated relative to wild type Fc are
boxed.)
                                                                                                    SEQ ID NO:15

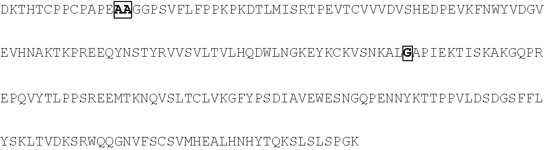

4A8-scFab-hFerritinLC
(Underlining indicates linker sequence; bolding indicates
hFerritinLC)
                                                                                                    SEQ ID NO:16
EIVMTQSPLSSPVTLGQPASISCRSSQSLVHSDGNTYLSWLQQRPGQPPRLLIYKISNRFSG
VPDRFSGSGAGTDFTLKISRVEAEDVGVYYCTQATQFPYTFGQGTKVDIKRTVAAPSVFIFP
PSDEQLKSGTASVVCLLNNFYPREAKVQWKVDNALQSGNSQESVTEQDSKDSTYSLSSTLTL
SKADYEKHKVYACEVTHQGLSSPVTKSFNRGECGGGGSGGGGSGGGGSGGGGSGGGGSGGGG
SGGGGSGGGGSGGGGSGGGGSGGGGSGGGGSGGGGSGGGGSEVQLVESGAEVKKPGASVKVS
CKVSGYTLTELSMHWVRQAPGKGLEWMGGFDPEDGETMYAQKFQGRVTMTEDTSTDTAYMEL
SSLRSEDTAVYYCATSTAVAGTPDLFDYYYGMDVWGQGTTVTVSSASTKGPSVFPLAPSSKS
TSGGTAALGCLVKDYFPEPVTVSWNSGALTSGVHTFPAVLQSSGLYSLSSVVTVPSSSLGTQ
TYICNVNHKPSNTKVDKKVEPKSCGGGGSGGGGSGGGGSGGGGSGGGGSGGSSQIRQNYSTD
VEAAVNSLVNLYLQASYTYLSLGFYFDRDDVALEGVSHFFRELAEEKREGYERLLKMQNQRG
GRALFQDIKKPAEDEWGKTPDAMKAAMALEKKLNQALLDLHALGSARTDPHLCDFLETHFLD
EEVKLIKKMGDHLTNLHRLGGPEAGLGEYLFERLTLRHD
V_{K} for 4A8
                                                                                                    SEQ ID NO:17
EIVMTQSPLSSPVTLGQPASISCRSSQSLVHSDGNTYLSWLQQRPGQPPRLLIYKISNRFSG
VPDRFSGSGAGTDFTLKISRVEAEDVGVYYCTQATQFPYTFGQGTKVDIK
V_{H} for 4A8
                                                                                                    SEQ ID NO:18
EVQLVESGAEVKKPGASVKVSCKVSGYTLTELSMHWVRQAPGKGLEWMGGFDPEDGETMYAQ
KFQGRVTMTEDTSTDTAYMELSSLRSEDTAVYYCATSTAVAGTPDLFDYYYGMDVWGQGTTV
TVSS
4A8-scFab C_hFerritinLC
(Underlining indicates linker sequence; bolding indicates
C_hFerritinLC)
                                                                                                    SEQ ID NO:19
EIVMTQSPLSSPVTLGQPASISCRSSQSLVHSDGNTYLSWLQQRPGQPPRLLIYKISNRFSG
VPDRFSGSGAGTDFTLKISRVEAEDVGVYYCTQATQFPYTFGQGTKVDIKRTVAAPSVFIFP
PSDEQLKSGTASVVCLLNNFYPREAKVQWKVDNALQSGNSQESVTEQDSKDSTYSLSSTLTL
SKADYEKHKVYACEVTHQGLSSPVTKSFNRGECGGGGSGGGGSGGGGSGGGGSGGGGSGGGG
SGGGGSGGGGSGGGGSGGGGSGGGGSGGGGSGGGGSGGGGSEVQLVESGAEVKKPGASVKVS
CKVSGYTLTELSMHWVRQAPGKGLEWMGGFDPEDGETMYAQKFQGRVTMTEDTSTDTAYMEL
SSLRSEDTAVYYCATSTAVAGTPDLFDYYYGMDVWGQGTTVTVSSASTKGPSVFPLAPSSKS
TSGGTAALGCLVKDYFPEPVTVSWNSGALTSGVHTFPAVLQSSGLYSLSSVVTVPSSSLGTQ
TYICNVNHKPSNTKVDKKVEPKSCGGGGSGGGGSGGGGSGGGGSGGGGSGGGKTPDAMKAAM
ALEKKLNQALLDLHALGSARTDPHLCDFLETHFLDEEVKLIKKMGDHLTNLHRLGGPEAGLG
EYLFERLTLRHD
mFerritin
                                                                                                    SEQ ID NO:20
MTSQIRQNYSTEVEAAVNRLVNLHLRASYTYLSLGFFFDRDDVALEGVGHFFRELAEEKREG
AERLLEFQNDRGGRALFQDVQKPSQDEWGKTQEAMEAALAMEKNLNQALLDLHALGSARTDP
HLCDFLESHYLDKEVKLIKKMGNHLTNLRRVAGPQPAQTGAPQGSLGEYLFERLTLKHD
HD37-scIgG
(underlining indicates linker sequence)
                                                                                                    SEQ ID NO:21
DILLTQTPASLAVSLGQRATISCKASQSVDYDGDSYLNWYQQIPGQPPKLLIYDASNLVSGI
PPRFSGSGSGTDFTLNIHPVEKVDAATYHCQQSTEDPWTFGGGTKLEIKRADAAPTVSIFPP
SSEQLTSGGASVVCFLNNFYPKDINVKWKIDGSERQNGVLNSWTDQDSKDSTYSMSSTLTLT
KDEYERHNSYTCEATHKTSTSPIVKSFNRNECGGSSGSGSGSTGTSSSGTGTSAGTTGTSAS
TSGSGSGGGGGSGGGGSAGGTATAGASSGSGSSGSSSSGGTGTGQVQLQQSGAELVRPGSSV
KISCKASGYAFSSYWMNWVKQRPGQGLEWIGQIWPGDGDTNYNGKFKGKATLTADESSSTAY
MQLSSLASEDSAVYFCARRETTTVGRYYYAMDYWGQGTSVTVSSSQSFPNVFPLVSCESPLS
DKNLVAMGCLARDFLPSTISFTWNYQNNTEVIQGIRTFPTLRTGGKYLATSQVLLSPKSILE
GSDEYLVCKIHYGGKNRDLHVPIPSKPCPPCKCPAPNLLGGPSVFIFPPKIKDVLMISLSPI
VTCVVVDVSEDDPDVQISWFVNNVEVHTAQTQTHREDYNSTLRVVSALPIQHQDWMSGKEFK
CKVNNKDLPAPIERTISKPKGSVRAPQVYVLPPPEEEMTKKQVTLTCMVTDFMPEDIYVEWT
NNGKTELNYKNTEPVLDSDGSYFMYSKLRVEKKNWVERNSYSCSVVHEGLHNHHTTKSFSRT
PGK
IgG2a Fc_mFerr
(underlining indicates linker sequence; bolding indicates
mFerritin)
                                                                                                    SEQ ID NO:22
KPCPPCKCPAPNLLGGPSVFIFPPKIKDVLMISLSPIVTCVVVDVSEDDPDVQISWFVNNVE
VHTAQTQTHREDYNSTLRVVSALPIQHQDWMSGKEFKCKVNNKDLPAPIERTISKPKGSVRA
PQVYVLPPPEEEMTKKQVTLTCMVTDFMPEDIYVEWTNNGKTELNYKNTEPVLDSDGSYFMY
SKLRVEKKNWVERNSYSCSVVHEGLHNHHTTKSFSRTPGKGGSSGSGSGSTGTSSSGTGTSA
GTTGTSASTSGSGSGGGGGSGGGGSAGGTATAGASSGSGSSGSSSSGGTGKPCPPCKCPAPN
LLGGPSVFIFPPKIKDVLMISLSPIVTCVVVDVSEDDPDVQISWFVNNVEVHTAQTQTHRED
YNSTLRVVSALPIQHQDWMSGKEFKCKVNNKDLPAPIERTISKPKGSVRAPQVYVLPPPEEE
MTKKQVTLTCMVTDFMPEDIYVEWTNNGKTELNYKNTEPVLDSDGSYFMYSKLRVEKKNWVE
RNSYSCSVVHEGLHNHHTTKSFSRTPGKSRASTASSASSGGGGGGSGGSGGSGGSMTSQIRQ
NYSTEVEAAVNRLVNLHLRASYTYLSLGFFFDRDDVALEGVGHFFRELAEEKREGAERLLEF
QNDRGGRALFQDVQKPSQDEWGKTQEAMEAALAMEKNLNQALLDLHALGSARTDPHLCDFLE
SHYLDKEVKLIKKMGNHLTNLRRVAGPQPAQTGAPQGSLGEYLFERLTLKHD
scFc-N-hFerr LALAP I253A
(Underlining indicates linker sequence; bolding indicates
hFerritinLC; boxes indicate residues that aremutated relative
to wild type IgG1 Fc)
                                                                                                    SEQ ID NO:23

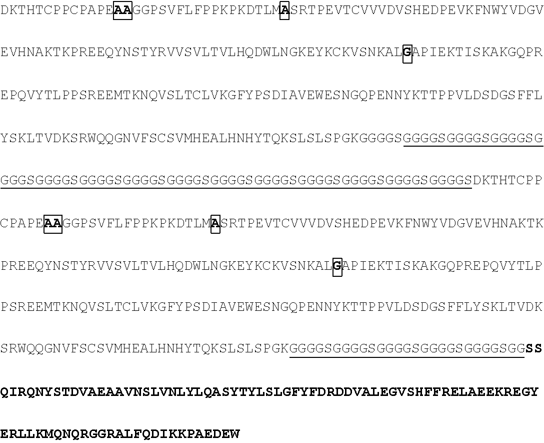

wild type human IgG1 Fc
                                                                                                    SEQ ID NO:24
DKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
Antibody 56 light chain
                                                                                                    SEQ ID NO:25
DIQMTQSPSSLSASVGDRVTITCRASQGISSYLAWYQQKPGKAPKLLIYDASNLQSGVPSRF
SGSGSGTDFTLTISSLQPEDFATYYCQQANSFPSTFGQGTKVEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
Antibody 56 heavy chain
                                                                                                    SEQ ID NO:26
QVQLVQSGAEVKKPGASVKVSCKASGYTFTSYGISWVRQAPGQGLEWMGWISAYNGNTNYAQ
KLQGRVTMTRDTSTSTVYMELSSLRSEDTAVYYCARDIGPIDYWGQGTLVTVSSASTKGPSV
FPLAPSSKSTSGGTAALGCLVKDYFPEPVTVSWNSGALTSGVHTFPAVLQSSGLYSLSSWVT
VPSSSLGTQTYICNVNHKPSNTKVDKKVEPKSC
Antibody 349 light chain
                                                                                                    SEQ ID NO:27
DIQMTQSPSSLSASVGDRVTITCRASQSISSWLAWYQQKPGKAPKLLIYDTSNLETGVPSRF
SGSGSGTDFTLTISSLQPEDFATYYCQQSYTTPWTFGQGTRLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
Antibody 349 heavy chain
                                                                                                    SEQ ID NO:28
EVQLLESGGGLVQPGGSLRLSCAASGFTFSNYGMHWVRQAPGKGLEWVSGISSAGSITNYAD
SVKGRFTISRDNSKNTLYLQMNSLRAEDTAVYYCAGNHAGTTVTSEYFQHWGQGTLVTVSSA
STKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSC
Antibody 178 light chain
                                                                                                    SEQ ID NO:29
EIVMTQSPATLSVSPGERATLSCKASQSVSGTYLAWYQQKPGQAPRLLIYGASTRATGIPAR
FSGSGSGTEFTLTISSLQSEDFAVYYCLQTHSYPPTFGQGTKVEIKRTVAAPSVFIFPPSDE
QLKSGTASVVCLLNNFYPREAKVQWKVDNALQSGNSQESVTEQDSKDSTYSLSSTLTLSKAD
YEKHKVYACEVTHQGLSSPVTKSFNRGEC
Antibody 178 heavy chain
                                                                                                    SEQ ID NO:30
QVQLVQSGAEVKKPGASVKVSCKASGYTFTDYHMHWVRQAPGQGLEWMGWINPNSGGTNYAQ
KFQGRVTMTRDTSTSTVYMELSSLRSEDTAVYYCARDISSWYEITKFDPWGQGTLVTVSSAS
TKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSC
Antibody 108 light chain
                                                                                                    SEQ ID NO:31
DIQMTQSPSSLSASVGDRVTITCRASQVITNNLAWYQQKPGKAPKLLIYDASTLETGVPSRF
SGSGSGTDFTLTISSLQPEDFATYYCQQSYTFPYTFGQGTKVEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
Antibody 108 heavy chain
                                                                                                    SEQ ID NO:32
QVQLVQSGAEVKKPGASVKVSCKASGYIFSRYAIHWVRQAPGQGLEWMGWMNPISGNTDYAP
NFQGRVTMTRDTSTSTVYMELSSLRSEDTAVYYCAKDGSQLAYLVEYFQHWGQGTLVTVSSA
STKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSC
Antibody 128 light chain
                                                                                                    SEQ ID NO:33
DIQMTQSPSSLSASVGDRVTITCRASQNISRYLNWYQQKPGKAPKLLIYDASNLETGVPSRF
SGSGSGTDFTLTISSLQPEDFATYYCQQANGFPPTFGQGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
Antibody 128 heavy chain
                                                                                                    SEQ ID NO:34
QVQLVQSGAEVKKPGASVKVSCKASGYTFTHYYMHWVRQAPGQGLEWMGIINPSSSSASYSQ
KFQGRVTMTRDTSTSTVYMELSSLRSEDTAVYYCARDGRYGSGSYPFDYWGQGTLVTVSSAS
TKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSC
Antibody 160 light chain
                                                                                                    SEQ ID NO:35
DIQMTQSPSSLSASVGDRVTITCRASQSVSSWLAWYQQKPGKAPKLLIYAASSLQSGVPSRF
SGSGSGTDFTLTISSLQPEDFATYYCQQGYTTPYTFGQGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
Antibody 160 heavy chain
                                                                                                    SEQ ID NO:36
QVQLVQSGAEVKKPGASVKVSCKASGYTFTGHDMHWVRQAPGQGLEWMGIINPSGGSTSYAQ
KFQGRVTMTRDTSTSTVYMELSSLRSEDTAVYYCARANSLRYYYGMDVWGQGTMVTVSSAST
KGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSC
Antibody 368 light chain
                                                                                                    SEQ ID NO:37
DIVMTQSPLSLPVTPGEPASISCRSSQSLLHSNGYNYLDWYLQKPGQSPQLLIYLGSNRASG
VPDRFSGSGSGTDFTLKISRVEAEDVGVYYCMQALQTPATFGPGTKVDIKRTVAAPSVFIFP
PSDEQLKSGTASVVCLLNNFYPREAKVQWKVDNALQSGNSQESVTEQDSKDSTYSLSSTLTL
SKADYEKHKVYACEVTHQGLSSPVTKSFNRGEC
Antibody 368 heavy chain
                                                                                                    SEQ ID NO:38
QVQLVQSGAEVKKPGSSVKVSCKASGYTFTSYDINWVRQAPGQGLEWMGAIMPMFGTANYAQ
KFQGRVTITADESTSTAYMELSSLRSEDTAVYYCARGSSGYYYGWGQGTLVTVSSASTKGPS
VFPLAPSSKSTSGGTAALGCLVKDYFPEPVTVSWNSGALTSGVHTFPAVLQSSGLYSLSVVS
TVPSSSLGTQTYICNVNHKPSNTKVDKKVEPKSC
Antibody 192 light chain
                                                                                                    SEQ ID NO:39
DIVMTQSPLSLPVTPGEPASISCRSSQSLLHSNGYNYLDWYLQKPGQSPQLLIYAASSLQSG
VPDRFSGSGSGTDFTLKISRVEAEDVGVYYCMQALQTPYTFGQGTKLEIKRTVAAPSVFIFP
PSDEQLKSGTASVVCLLNNFYPREAKVQWKVDNALQSGNSQESVTEQDSKDSTYSLSSTLTL
SKADYEKHKVYACEVTHQGLSSPVTKSFNRGEC
Antibody 192 heavy chain
                                                                                                    SEQ ID NO:40
QVQLVQSGAEVKKPGSSVKVSCKASGGTFSSYAISWVRQAPGQGLEWMGWINPNSGGANYAQ
KFQGRVTITADESTSTAYMELSSLRSEDTAVYYCSTYYYDSSGYSTDYWGQGTLVTVSSAST
KGPSVFPLAPSSKSTSGGTAALGCLVKDYFPEPVTVSWNSGALTSGVHTFPAVLQSSGLYSL
SSWTVPSSSLGTQTYICNVNHKPSNTKVDKKVEPKSC
Antibody 158 light chain
                                                                                                    SEQ ID NO:41
DIQMTQSPSSLSASVGDRVTITCRASQSISRYLNWYQQKPGKAPKLLIYDASNLESGVPSRF
SGSGSGTDFTLTISSLQPEDFATYYCQQANSFPLTFGGGTKVDIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
Antibody 158 heavy chain
                                                                                                    SEQ ID NO:42
QVQLVQSGAEVKKPGASVKVSCKASGYTFTGYYMHWVRQAPGQGLEWMGWINPLNGGTNFAP
KFQGRVTMTRDTSTSTVYMELSSLRSEDTAVYYCARDPGGSYSNDAFDIWGQGTLVTVSSAS
TKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSC
Antibody 180 light chain
                                                                                                    SEQ ID NO:43
DIVMTQSPLSLPVTPGEPASISCRSSQSLLHSNGYNYLDWYLQKPGQSPQLLIYAASSLQSG
VPDRFSGSGSGTDFTLKISRVEAEDVGVYYCQQYYSSPYTFGQGTKLEIKRTVAAPSVFIFP
PSDEQLKSGTASVVCLLNNFYPREAKVQWKVDNALQSGNSQESVTEQDSKDSTYSLSSTLTL
SKADYEKHKVYACEVTHQGLSSPVTKSFNRGEC
Antibody 180 heavy chain
                                                                                                    SEQ ID NO:44
QVQLVQSGAEVKKPGSSVKVSCKASGYTFTSYAMHWVRQAPGQGLEWMGRISPRSGGTKYAQ
RFQGRVTITADESTSTAYMELSSLRSEDTAVYYCAREAVAGTHPQAGDFDLWGRGTLVTVSS
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSC
Antibody 254 light chain
                                                                                                    SEQ ID NO:45
DIQMTQSPSSLSASVGDRVTITCRASQGISSYLAWYQQKPGKAPKLLIYDASSLQIGVPSRF
SGSGSGTDFTLTISSLQPEDFATYYCLQSYSTPPWTFGQGTKVEIKRTVAAPSVFIFPPSDE
QLKSGTASVVCLLNNFYPREAKVQWKVDNALQSGNSQESVTEQDSKDSTYSLSSTLTLSKAD
YEKHKVYACEVTHQGLSSPVTKSFNRGEC
Antibody 254 heavy chain
                                                                                                    SEQ ID NO:46
EVQLLESGGGLVQPGGSLRLSCAASGFTFSSSAMHWVRQAPGKGLEWVSAIGTGGDTYYADS
VKGRFTISRDNSKNTLYLQMNSLRAEDTAVYYCAREGDGYNFYFDYWGQGTLVTVSSASTKG
PSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSC
Antibody 120 light chain
                                                                                                    SEQ ID NO:47
EIVMTQSPATLSVSPGERATLSCRASQSVSSRYLAWYQQKPGQAPRLLIYGASTRATGIPAR
FSGSGSGTEFTLTISSLQSEDFAVYYCQQYYTTPRTFGQGTRLEIKRTVAAPSVFIFPPSDE
QLKSGTASVVCLLNNFYPREAKVQWKVDNALQSGNSQESVTEQDSKDSTYSLSSTLTLSKAD
YEKHKVYACEVTHQGLSSPVTKSFNRGEC
Antibody 120 heavy chain
                                                                                                    SEQ ID NO:48
QVQLVQSGAEVKKPGASVKVSCKASGYTFTSYDINWVRQAPGQGLEWMGMIDPSGGSTSYAQ
KFQGRVTMTRDTSTSTVYMELSSLRSEDTAVYYCAKDFGGGTRYDYWYFDLWGRGTLVTVSS
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSC
Antibody 64 light chain
                                                                                                    SEQ ID NO:49
DIQMTQSPSSLSASVGDRVTITCRASQGISSHLAWYQQKPGKAPKLLIYDASNLETGVPSRF
SGSGSGTDFTLTISSLQPEDFATYYCQQTYSTPWTFGQGTKVEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
Antibody 64 heavy chain
                                                                                                    SEQ ID NO:50
EVQLLESGGGLVQPGGSLRLSCAASGFPFSQHGMHWVRQAPGKGLEWVSAIDRSGSYIYYAD
SVKGRFTISRDNSKNTLYLQMNSLRAEDTAVYYCARDTYGGKVTYFDYWGQGTLVTVSSAST
KGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSC
Antibody 298 light chain
                                                                                                    SEQ ID NO:51
DIVMTQSPDSLAVSLGERATINCKSSQSVLYSSNNKNYLAWYQQKPGQPPKLLIYWASTRES
GVPDRFSGSGSGTDFTLTISSLQAEDVAVYYCQQYYSTPPTFGQGTKLEIKRTVAAPSVFIF
PPSDEQLKSGTASVVCLLNNFYPREAKVQWKVDNALQSGNSQESVTEQDSKDSTYSLSSTLT
LSKADYEKHKVYACEVTHQGLSSPVTKSFNRGEC
Antibody 298 heavy chain
                                                                                                    SEQ ID NO:52
QVQLVQSGAEVKKPGASVKVSCKASGGTFSTYGISWVRQAPGQGLEWMGWISPNSGGTDLAQ
KFQGRVTMTRDTSTSTVYMELSSLRSEDTAVYYCASDPRDDIAGGYWGQGTLVTVSSASTKG
PSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSC
Antibody 82 light chain
                                                                                                    SEQ ID NO:53
DIQMTQSPSSLSASVGDRVTITCRASQVISNYLAWYQQKPGKAPKLLIYDASNLETGVPSRF
SGSGSGTDFTLTISSLQPEDFATYYCQQSFSPPPTFGQGTRLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
Antibody 82 heavy chain
                                                                                                    SEQ ID NO:54
QVQLVQSGAEVKKPGASVKVSCKASGGSFSTSAFYWVRQAPGQGLEWMGWINPYTGGTNYAQ
KFQGRVTMTRDTSTSTVYMELSSLRSEDTAVYYCARSRALYGSGSYFDYWGQGTLVTVSSAS
TKGPSVFPLAPSSKSTSGGTAALGCLVKDYFPEPVTVSWNSGALTSGVHTFPAVLQSSGLYS
LSSWVTVPSSSLGTQTYICNVNHKPSNTKVDKKVEPKSC
Antibody 46 light chain
                                                                                                    SEQ ID NO:55
DIQMTQSPSSLSASVGDRVTITCRASQSISSWLAWYQQKPGKAPKLLIYDASNLETGVPSRF
SGSGSGTDFTLTISSLQPEDFATYYCQQSYSTPFTFGPGTKVDIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
Antibody 46 heavy chain
                                                                                                    SEQ ID NO:56
EVQLLESGGGLVQPGRSLRLSCAASGFTFSSYAMSWVRQAPGKGLEWVSTIYSGGSTYYADS
VKGRFTISRDNSKNTLYLQMNSLRAEDTAVYYCARGDSRDAFDIWGQGTMVTVSSASTKGPS
VFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSC
Antibody 324 light chain
                                                                                                    SEQ ID NO:57
DIQMTQSPSSLSASVGDRVTITCRASQSITTYLNWYQQKPGKAPKLLIYDASNLETGVPSRF
SGSGSGTDFTLTISSLQPEDFATYYCQQSYSTPPTFGQGTKVEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
Antibody 324 heavy chain
                                                                                                    SEQ ID NO:58
QVQLVQSGAEVKKPGASVKVSCKASGGTFNNYGISWVRQAPGQGLEWMGWMNPNSGNTGYAQ
KFQGRVTMTRDTSTSTVYMELSSLRSEDTAVYYCARVGDYGDYIVSPFDLWGRGTLVTVSSA
STKGPSVFPLAPSSKSTSGGTAALGCLVKDYFPEPVTVSWNSGALTSGVHTFPAVLQSSGLY
SLSSWTVPSSSLGTQTYICNVNHKPSNTKVDKKVEPKSC
Antibody 236 light chain
                                                                                                    SEQ ID NO:59
DIVMTQSPLSLPVTPGEPASISCRSSQSLLHSNGYNYLDWYLQKPGQSPQLLIYLGSNRASG
VPDRFSGSGSGTDFTLKISRVEAEDVGVYYCMQALQTPPTFGQGTRLEIKRTVAAPSVFIFP
PSDEQLKSGTASVVCLLNNFYPREAKVQWKVDNALQSGNSQESVTEQDSKDSTYSLSSTLTL
SKADYEKHKVYACEVTHQGLSSPVTKSFNRGEC
Antibody 236 heavy chain
                                                                                                    SEQ ID NO:60
QVQLVQSGAEVKKPGASVKVSCKASGGTFTSYGINWVRQAPGQGLEWMGWMNPNSGNTGYAQ
KFQGRVTMTRDTSTSTVYMELSSLRSEDTAVYYCASRGIQLLPRGMDVWGQGTTVTVSSAST
KGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSC
Antibody 52 light chain
                                                                                                    SEQ ID NO:61
DIQMTQSPSSLSASVGDRVTITCRASQGISNNLNWYQQKPGKAPKLLIYAASSLESGVPSRF
SGSGSGTDFTLTISSLQPEDFATYYCQQGNGFPLTFGPGTKVDIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
Antibody 52 light chain N92T
                                                                                                    SEQ ID NO:62

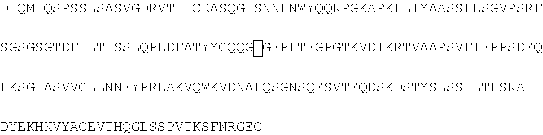

Antibody 52 heavy chain
                                                                                                    SEQ ID NO:63
QVQLVQSGAEVKKPGSSVKVSCKASGYTFTSYGISWVRQAPGQGLEWMGGIIPMFGTTNYAQ
KFQGRVTITADKSTSTAYMELSSLRSEDTAVYYCARDRGDTIDYWGQGTLVTVSSASTKGPS
VFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSC
Antibody 80 light chain
                                                                                                    SEQ ID NO:64
DIVMTQSPDSLAVSLGERATINCKSSQSVLYSSNNKNYLAWYQQKPGQPPKLLIYWASTRES
GVPDRFSGSGSGTDFTLTISSLQAEDVAVYYCQQYYSAPLTFGGGTKVEIKRTVAAPSVFIF
PPSDEQLKSGTASVVCLLNNFYPREAKVQWKVDNALQSGNSQESVTEQDSKDSTYSLSSTLT
LSKADYEKHKVYACEVTHQGLSSPVTKSFNRGEC
Antibody 80 heavy chain
                                                                                                    SEQ ID NO:65
QVQLVQSGAEVKKPGSSVKVSCKASGGTFNRYAFSWVRQAPGQGLEWMGGIIPIFGTANYAQ
KFQGRVTITADESTSTAYMELSSLRSEDTAVYYCARSTRELPEVVDWYFDLWGRGTLVTVSS
ASTKGPSVFPLAPSSKSTSGGTAALGCLVKDYFPEPVTVSWNSGALTSGVHTFPAVLQSSGL
YSLSSWTVPSSSLGTQTYICNVNHKPSNTKVDKKVEPKSC
```

### EQUIVALENTS/OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features herein before set forth.

1. A fusion protein comprising a nanocage monomer linked to a SARS-CoV-2 binding moiety, wherein a plurality of the fusion proteins self-assemble to form a nanocage.

2. The fusion protein of claim 1, wherein the SARS-CoV-2 binding moiety targets the SARS-CoV-2 S glycoprotein.

3. (canceled)

4. The fusion protein of claim 1, wherein the SARS-CoV-2 binding moiety comprises an antibody or fragment thereof.

5. The fusion protein of claim 4, wherein the antibody or fragment thereof comprises a Fab fragment.

6. The fusion protein of claim 4, wherein the antibody or fragment thereof comprises a scFab fragment, a scFv fragment, a sdAb fragment, a VHH domain or a combination thereof.

7. The fusion protein of claim 4, wherein the antibody or fragment thereof comprises a heavy and/or light chain of a Fab fragment.

8. The fusion protein of claim 4, wherein the SARS-CoV-2 binding moiety comprises single chain variable domain VHH-72, BD23 and/or 4A8.

9. The fusion protein of claim 4, wherein the SARS-CoV-2 binding moiety comprises an mAb listed in Table 4.

10. The fusion protein of claim 9, wherein the SARS-CoV-2 binding moiety comprises mAb 298, 324, 46, 80, 52, 82, or 236 from Table 4, or variants thereof.

11. The fusion protein of claim 1, wherein the SARS-CoV-2 binding moiety is linked at the N- or C-terminus of the nanocage monomer, or wherein there is a first SARS-CoV-2 binding moiety linked at the N-terminus and a second SARS-CoV-2 binding moiety linked at the C-terminus of the nanocage monomer, wherein the first and second SARS-CoV-2 binding moieties are the same or different.

12. The fusion protein of claim 1, wherein the nanocage monomer comprises a first nanocage monomer subunit linked to the SARS-CoV-2 binding moiety; wherein the first nanocage monomer subunit self-assembles with a second nanocage monomer subunit to form the nanocage monomer.

13-20. (canceled)

21. The fusion protein of claim 1, wherein the nanocage monomer is selected from ferritin, apoferritin, encapsulin, SOR, lumazine synthase, pyruvate dehydrogenase, carboxysome, vault proteins, GroEL, heat shock protein, E2P, MS2 coat protein, fragments thereof, and variants thereof.

22. The fusion protein of claim 21, wherein the nanocage monomer is apoferritin.

23. The fusion protein of claim 22, wherein the first and second nanocage monomer subunits interchangeably comprise the “N” and “C” regions of apoferritin.

24.-25. (canceled)

26. The fusion protein of claim 1, further comprising a linker between the nanocage monomer subunit and the bioactive moiety.

27. The fusion protein of claim 26, wherein the linker is flexible or rigid and comprises from about 1 to about 30 amino acid residues.

28. The fusion protein of claim 26, wherein the linker comprises at least one GGS repeat.

29. (canceled)

30. The fusion protein of claim 1, further comprising a C-terminal linker.

31. (canceled)

32. A nanocage comprising at least one fusion protein of claim 1 and at least one second nanocage monomer subunit that self-assembles with the fusion protein to form a nanocage monomer.

33. The nanocage of claim 32, wherein each nanocage monomer comprises the fusion protein of claim 1.

34. (canceled)

35. The nanocage of claim 32, comprising at least 2, 3, 4, 5, 6, 7, 8, 9, or 10 different SARS-CoV-2 binding moieties.

36. The nanocage of claim 32, wherein the nanocage is multivalent and/or multispecific.

37. The nanocage of claim 32, comprising one of more mAbs from Table 4.

38. The nanocage of claim 37, comprising 3 mAbs from Table 4.

39. The nanocage of claim 37 comprising mAbs 298, 324, 46, 52, 80, 82 and/or 236 from Table 4.

40. The nanocage of claim 32, comprising a 4:2:1:1 ratio of scFab1-human apoferritin:scFc-human N-Ferritin:scFab2-C-Ferritin:scFab3-C-Ferritin.

41. The nanocage of claim 32, carrying a cargo molecule selected from the group consisting of a pharmaceutical agent, a diagnostic agent, and an imaging agent.

42.-44. (canceled)

45. A tri-specific antibody construct targeting SARS-CoV-2.

46. A SARS-CoV-2 therapeutic or prophylactic composition comprising the nanocage of claim 32.

47. A nucleic acid molecule encoding the fusion protein of claim 1.

48. A vector comprising the nucleic acid molecule of claim 47.

49. A host cell comprising the vector of claim 48.

50. A method for treating and/or preventing SARS-CoV-2 in a subject in need thereof, the method comprising administering to the subject the nanocage of claim 32.

51.-52. (canceled)

53. A polypeptide comprising an amino acid sequence having at least

| | |
|---|---|
| QVQLVQSGAEVKKPGASVKVSCKASG | DIQMTQSPSSLSASVGDRVTITCRASQG |
| YTFTSYGISWVRQAPGQGLEWMGWIS | ISSYLAWYQQKPGKAPKLLIYDASNLQ |
| AYNGNTNYAQKLQGRVTMTRDTSTST | SGVPSRFSGSGSGTDFTLTISSLQPEDFA |
| VYMELSSLRSEDTAVYYCARDIGPIDY | TYYCQQANSFPSTFGQGTKVEIKR |
| WGQGTLVTVSS | (SEQ ID NO: 88) |
| (SEQ ID NO: 68) | |
| EVQLLESGGGLVQPGGSLRLSCAASGF | DIQMTQSPSSLSASVGDRVTITCRASQS |
| TFSNYGMHWVRQAPGKGLEWVSGISS | ISSWLAWYQQKPGKAPKLLIYDTSNLE |
| AGSITNYADSVKGRFTISRDNSKNTLY | TGVPSRFSGSGSGTDFTLTISSLQPEDF |
| LQMNSLRAEDTAVYYCAGNHAGTTV | ATYYCQQSYTTPWTFGQGTRLEIKR |
| TSEYFQHWGQGTLVTVSS | (SEQ ID NO: 89) |
| (SEQ ID NO: 69) | |
| QVQLVQSGAEVKKPGASVKVSCKASG | EIVMTQSPATLSVSPGERATLSCKASQS |
| YTFTDYHMHWVRQAPGQGLEWMGWI | VSGTYLAWYQQKPGQAPRLLIYGAST |
| NPNSGGTNYAQKFQGRVTMTRDTSTS | RATGIPARFSGSGSGTEFTLTISSLQSED |
| TVYMELSSLRSEDTAVYYCARDISSWY | FAVYYCLQTHSYPPTFGQGTKVEIKR |
| EITKFDPWGQGTLVTVSS | (SEQ ID NO: 90) |
| (SEQ ID NO: 70) | |
| QVQLVQSGAEVKKPGASVKVSCKASG | DIQMTQSPSSLSASVGDRVTITCRASQV |
| YIFSRYAIHWVRQAPGQGLEWMGWM | ITNNLAWYQQKPGKAPKLLIYDASTLE |
| NPISGNTDYAPNFQGRVTMTRDTSTST | TGVPSRFSGSGSGTDFTLTISSLQPEDF |
| VYMELSSLRSEDTAVYYCAKDGSQLA | ATYYCQQSYTFPYTFGQGTKVEIKR |
| YLVEYFQHWGQGTLVTVSS | (SEQ ID NO: 91) |
| (SEQ ID NO: 71) | |
| QVQLVQSGAEVKKPGASVKVSCKASG | DIQMTQSPSSLSASVGDRVTITCRASQN |
| YTFTHYYMHWVRQAPGQGLEWMGII | ISRYLNWYQQKPGKAPKLLIYDASNLE |
| NPSSSSASYSQKFQGRVTMTRDTSTST | TGVPSRFSGSGSGTDFTLTISSLQPEDF |
| VYMELSSLRSEDTAVYYCARDGRYGS | ATYYCQQANGFPPTFGQGTKLEIKR |
| GSYPFDYWGQGTLVTVSS | (SEQ ID NO: 92) |
| (SEQ ID NO: 72) | |
| QVQLVQSGAEVKKPGASVKVSCKASG | DIQMTQSPSSLSASVGDRVTITCRASQS |
| YTFTGHDMHWVRQAPGQGLEWMGII | VSSWLAWYQQKPGKAPKLLIYAASSL |
| NPSGGSTSYAQKFQGRVTMTRDTSTST | QSGVPSRFSGSGSGTDFTLTISSLQPEDF |
| VYMELSSLRSEDTAVYYCARANSLRY | ATYYCQQGYTTPYTFGQGTKLEIKR |
| YYGMDVWGQGTMVTVSS | (SEQ ID NO: 93) |
| (SEQ ID NO: 73) | |
| QVQLVQSGAEVKKPGSSVKVSCKASG | DIVMTQSPLSLPVTPGEPASISCRSSQSL |
| YTFTSYDINWVRQAPGQGLEWMGAIM | LHSNGYNYLDWYLQKPGQSPQLLIYL |
| PMFGTANYAQKFQGRVTITADESTSTA | GSNRASGVPDRFSGSGSGTDFTLKISRV |
| YMELSSLRSEDTAVYYCARGSSGYYY | EAEDVGVYYCMQALQTPATFGPGTKV |
| GWGQGTLVTVSS | DIKR |
| (SEQ ID NO: 74) | (SEQ ID NO: 94) |
| QVQLVQSGAEVKKPGSSVKVSCKASG | DIVMTQSPLSLPVTPGEPASISCRSSQSL |
| GTFSSYAISWVRQAPGQGLEWMGWIN | LHSNGYNYLDWYLQKPGQSPQLLIYA |
| PNSGGANYAQKFQGRVTITADESTSTA | ASSLQSGVPDRFSGSGSGTDFTLKISRV |
| YMELSSLRSEDTAVYYCSTYYYDSSG | EAEDVGVYYCMQALQTPYTFGQGTKL |
| YSTDYWGQGTLVTVSS | EIKR |
| (SEQ ID NO: 75) | (SEQ ID NO: 95) |
| QVQLVQSGAEVKKPGASVKVSCKASG | DIQMTQSPSSLSASVGDRVTITCRASQS |
| YTFTGYYMHWVRQAPGQGLEWMGWI | ISRYLNWYQQKPGKAPKLLIYDASNLE |
| NPLNGGTNFAPKFQGRVTMTRDTSTST | SGVPSRFSGSGSGTDFTLTISSLQPEDFA |
| VYMELSSLRSEDTAVYYCARDPGGSY | TYYCQQANSFPLTFGGGTKVDIKR |
| SNDAFDIWGQGTLVTVSS | (SEQ ID NO: 96) |
| (SEQ ID NO: 76) | |
| QVQLVQSGAEVKKPGSSVKVSCKASG | DIVMTQSPLSLPVTPGEPASISCRSSQSL |
| YTFTSYAMHWVRQAPGQGLEWMGRI | LHSNGYNYLDWYLQKPGQSPQLLIYA |
| SPRSGGTKYAQRFQGRVTITADESTST | ASSLQSGVPDRFSGSGSGTDFTLKISRV |
| AYMELSSLRSEDTAVYYCAREAVAGT | EAEDVGVYYCQQYYSSPYTFGQGTKL |
| HPQAGDFDLWGRGTLVTVSS | EIKR |
| (SEQ ID NO: 77) | (SEQ ID NO: 97) |
| EVQLLESGGGLVQPGGSLRLSCAASGF | DIQMTQSPSSLSASVGDRVTITCRASQG |
| TFSSSAMHWVRQAPGKGLEWVSAIGT | ISSYLAWYQQKPGKAPKLLIYDASSLQI |
| GGDTYYADSVKGRFTISRDNSKNTLYL | GVPSRFSGSGSGTDFTLTISSLQPEDFA |
| QMNSLRAEDTAVYYCAREGDGYNFYF | TYYCLQSYSTPPWTFGQGTKVEIKR |
| DYWGQGTLVTVSS | (SEQ ID NO: 98) |
| (SEQ ID NO: 78) | |
| QVQLVQSGAEVKKPGASVKVSCKASG | EIVMTQSPATLSVSPGERATLSCRASQS |
| YTFTSYDINWVRQAPGQGLEWMGMID | VSSRYLAWYQQKPGQAPRLLIYGAST |
| PSGGSTSYAQKFQGRVTMTRDTSTSTV | RATGIPARFSGSGSGTEFTLTISSLQSED |
| YMELSSLRSEDTAVYYCAKDFGGGTR | FAVYYCQQYYTTPRTFGQGTRLEIKR |
| YDYWYFDLWGRGTLVTVSS | (SEQ ID NO: 99) |
| (SEQ ID NO: 79) | |
| EVQLLESGGGLVQPGGSLRLSCAASGF | DIQMTQSPSSLSASVGDRVTITCRASQG |
| PFSQHGMHWVRQAPGKGLEWVSAIDR | ISSHLAWYQQKPGKAPKLLIYDASNLE |
| SGSYIYYADSVKGRFTISRDNSKNTLY | TGVPSRFSGSGSGTDFTLTISSLQPEDF |
| LQMNSLRAEDTAVYYCARDTYGGKV | ATYYCQQTYSTPWTFGQGTKVEIKR |
| TYFDYWGQGTLVTVSS | (SEQ ID NO: 100) |
| (SEQ ID NO: 80) | |
| QVQLVQSGAEVKKPGASVKVSCKASG | DIVMTQSPDSLAVSLGERATINCKSSQS |
| GTFSTYGISWVRQAPGQGLEWMGWIS | VLYSSNNKNYLAWYQQKPGQPPKLLI |
| PNSGGTDLAQKFQGRVTMTRDTSTST | YWASTRESGVPDRFSGSGSGTDFTLTIS |
| VYMELSSLRSEDTAVYYCASDPRDDIA | SLQAEDVAVYYCQQYYSTPPTFGQGT |
| GGYWGQGTLVTVSS | KLEIKR |
| (SEQ ID NO: 81) | (SEQ ID NO: 101) |
| QVQLVQSGAEVKKPGASVKVSCKASG | DIQMTQSPSSLSASVGDRVTITCRASQV |
| GSFSTSAFYWVRQAPGQGLEWMGWIN | ISNYLAWYQQKPGKAPKLLIYDASNLE |
| PYTGGTNYAQKFQGRVTMTRDTSTST | TGVPSRFSGSGSGTDFTLTISSLQPEDF |
| VYMELSSLRSEDTAVYYCARSRALYG | ATYYCQQSFSPPPTFGQGTRLEIKR |
| SGSYFDYWGQGTLVTVSS | (SEQ ID NO: 102) |
| (SEQ ID NO: 82) | |
| EVQLLESGGGLVQPGRSLRLSCAASGF | DIQMTQSPSSLSASVGDRVTITCRASQS |
| TFSSYAMSWVRQAPGKGLEWVSTIYS | ISSWLAWYQQKPGKAPKLLIYDASNLE |
| GGSTYYADSVKGRFTISRDNSKNTLYL | TGVPSRFSGSGSGTDFTLTISSLQPEDF |
| QMNSLRAEDTAVYYCARGDSRDAFDI | ATYYCQQSYSTPFTFGPGTKVDIKR |
| WGQGTMVTVSS | (SEQ ID NO: 103) |
| (SEQ ID NO: 83) | |
| QVQLVQSGAEVKKPGASVKVSCKASG | DIQMTQSPSSLSASVGDRVTITCRASQS |
| GTFNNYGISWVRQAPGQGLEWMGWM | ITTYLNWYQQKPGKAPKLLIYDASNLE |
| NPNSGNTGYAQKFQGRVTMTRDTSTS | TGVPSRFSGSGSGTDFTLTISSLQPEDF |
| TVYMELSSLRSEDTAVYYCARVGDYG | ATYYCQQSYSTPPTFGQGTKVEIKR |
| DYIVSPFDLWGRGTLVTVSS | (SEQ ID NO: 104) |
| (SEQ ID NO: 84) | |
| QVQLVQSGAEVKKPGASVKVSCKASG | DIVMTQSPLSLPVTPGEPASISCRSSQSL |
| GTFTSYGINWVRQAPGQGLEWMGWM | LHSNGYNYLDWYLQKPGQSPQLLIYL |
| NPNSGNTGYAQKFQGRVTMTRDTSTS | GSNRASGVPDRFSGSGSGTDFTLKISRV |
| TVYMELSSLRSEDTAVYYCASRGIQLL | EAEDVGVYYCMQALQTPPTFGQGTRL |
| PRGMDVWGQGTTVTVSS | EIKR |
| (SEQ ID NO: 85) | (SEQ ID NO: 105) |
| QVQLVQSGAEVKKPGSSVKVSCKASG | DIQMTQSPSSLSASVGDRVTITCRASQG |
| YTFTSYGISWVRQAPGQGLEWMGGIIP | ISNNLNWYQQKPGKAPKLLIYAASSLE |
| MFGTTNYAQKFQGRVTITADKSTSTA | SGVPSRFSGSGSGTDFTLTISSLQPEDFA |
| YMELSSLRSEDTAVYYCARDRGDTID | TYYCQQGNGFPLTFGPGTKVDIKR |
| YWGQGTLVTVSS | (SEQ ID NO: 106) |
| (SEQ ID NO: 86) | |
| QVQLVQSGAEVKKPGSSVKVSCKASG | DIVMTQSPDSLAVSLGERATINCKSSQS |
| GTFNRYAFSWVRQAPGQGLEWMGGII | VLYSSNNKNYLAWYQQKPGQPPKLLI |
| PIFGTANYAQKFQGRVTITADESTSTA | YWASTRESGVPDRFSGSGSGTDFTLTIS |
| YMELSSLRSEDTAVYYCARSTRELPEV | SLQAEDVAVYYCQQYYSAPLTFGGGT |
| VDWYFDLWGRGTLVTVSS | KVEIKR |
| (SEQ ID NO: 87) | (SEQ ID NO: 107) |

or a functional fragment thereof.

54.-56. (canceled)

57. A fusion polypeptide comprising (1) a fragment crystallizable (Fc) region linked to (2) a nanocage monomer or subunit thereof, wherein the Fc region comprises the I253A mutation, wherein numbering is according to the EU index.

58.-66. (canceled)

67. A self-assembled polypeptide complex comprising:
(a) a plurality of first fusion polypeptides, each first fusion polypeptide comprising (1) an Fc region linked to (2) a nanocage monomer or subunit thereof, and
(b) a plurality of second fusion polypeptides, each second fusion polypeptide comprising (1) a SARS-CoV-2-binding antibody fragment linked to (2) a nanocage monomer or subunit thereof.

68. The self-assembled polypeptide complex of claim 67, wherein the nanocage monomer is a ferritin monomer.

69. The self-assembled polypeptide complex of claim 68, wherein the nanocage monomer is a ferritin light chain.

70. The self-assembled polypeptide complex of claim 69, which does not comprise any ferritin heavy chains or subunits of ferritin heavy chains.

71.-82. (canceled)

83. A self-assembled polypeptide complex comprising:
(a) a plurality of first fusion polypeptides, each first fusion polypeptide comprising (1) an IgG1 Fc region linked to (2) a human ferritin monomer or subunit thereof, wherein the IgG1 Fc region comprises the LALAP (L234A/L235A/P329G) and I253A mutations, wherein numbering is according to the EU index, and
(b) a plurality of second fusion polypeptides, each second fusion polypeptide comprising (1) a Fab fragment of an antibody that is capable of binding to a SARS-CoV-2 protein, the Fab fragment being linked to (2) a human ferritin monomer or subunit thereof.

84-99. (canceled)

100. A method of treating, ameliorating, or preventing a SARS-CoV-2-related condition, the method comprising administering to a subject a composition comprising the self-assembled polypeptide complex of claim 67.

101.-102. (canceled)

103. The fusion protein of claim 10, wherein the SARS-CoV-2 binding moiety comprises mAb 298, 80, and 52 from Table 4, or variants thereof.
